SEQUENCE LISTING (156 sequences)

SEQ ID NO: 1 - protein, 271 aa, Artificial Sequence
  Recombinant polypeptide
MGILPSPGMPALLSLVSLLSVLLMGCVAETGTNLCPFGEVFNATRFASVYAWNRKNISNCVADYSVLYNSASFSTFKCYNVSPTNLTDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGSGGSGGSGGSGGSGGSKIFIMIVGGLIGLRIVFAVLSVIHRVR

SEQ ID NO: 2 - protein, 272 aa, Artificial Sequence
  Recombinant polypeptide
MGILPSPGMPALLSLVSLLSVLLMGCVAETGTNLCPFGEVFNATRFASVYAWNRKNISNCVADYSVLYNSASFSTFKCYNVSPTNLTDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGSAKFVAAWTLKAAAGGSKIFIMIVGGLIGLRIVFAVLSVIHRVR

SEQ ID NO: 3 - protein, 18 aa, Artificial Sequence
  Recombinant polypeptide
GGSGGSGGSGGSGGSGGS

SEQ ID NO: 4 - protein, 19 aa, Artificial Sequence
  Recombinant polypeptide
GGSAKFVAAWTLKAAAGGS

SEQ ID NO: 5 - protein, 31 aa, Artificial Sequence
  Recombinant polypeptide
MGILPSPGMPALLSLVSLLSVLLMGCVAETG

SEQ ID NO: 6 - protein, 27 aa, Artificial Sequence
  Recombinant polypeptide
KIFIMIVGGLIGLRIVFAVLSVIHRVR

SEQ ID NO: 7 - protein, 24 aa, Artificial Sequence
  Recombinant polypeptide
KWPWYIWLGFIAGLIAIVMVTIML

SEQ ID NO: 8 - protein, 22 aa, Artificial Sequence
  Recombinant polypeptide
KSSIASFFFIIGLIIGLFLVLR

SEQ ID NO: 9 - protein, 195 aa, Artificial Sequence [sequence omitted]

SEQ ID NO: 10 - protein, 194 aa, Artificial Sequence
  Recombinant polypeptide
TNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGP

SEQ ID NO: 11 - protein, 1273 aa, Artificial Sequence
  Recombinant polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1255 aa, Artificial Sequence
  Recombinant polypeptide
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 13 - protein, 195 aa, Artificial Sequence
  Recombinant polypeptide
  MISC_FEATURE (56)..(56): Xaa is Ser or Thr
TNLCPFGEVFNATRFASVYAWNRKNISNCVADYSVLYNSASFSTFKCYNVSPTNLXDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 14 - protein, 195 aa, Artificial Sequence
  Recombinant polypeptide
  MISC_FEATURE (188)..(188): Xaa is Ser or Thr
  MISC_FEATURE (192)..(192): Xaa is Ser or Thr
TNLCPFGEVFNATRFASVYAWNRKNISNCVADYSVLYNSASFSTFKCYGVSPTKLNDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHXPNTXCGP

SEQ ID NO: 15 - protein, 195 aa, Artificial Sequence
  Recombinant polypeptide
  MISC_FEATURE (64)..(64): Xaa is Ser or Thr
  MISC_FEATURE (188)..(188): Xaa is Ser or Thr
TNLCPFGEVFNATRFASVYAWNRKNISNCVADYSVLYNSASFSTFKCYGVSPTKLNDLCFTNVXADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHXPATVCGP

SEQ ID NO: 16 - protein, 195 aa, Artificial Sequence
  Recombinant polypeptide
  MISC_FEATURE (56)..(56): Xaa is Ser or Thr
  MISC_FEATURE (64)..(64): Xaa is Ser or Thr
  MISC_FEATURE (188)..(188): Xaa is Ser or Thr
TNLCPFGEVFNATRFASVYAWNRKNISNCVADYSVLYNSASFSTFKCYGVSPTNLXDLCFTNVXADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHXPATVCGP

SEQ ID NO: 17 - protein, 195 aa, Artificial Sequence
  Recombinant polypeptide
  MISC_FEATURE (56)..(56): Xaa is Ser or Thr
  MISC_FEATURE (64)..(64): Xaa is Ser or Thr
  MISC_FEATURE (188)..(188): Xaa is Ser or Thr
  MISC_FEATURE (192)..(192): Xaa is Ser or Thr
TNLCPFGEVFNATRFASVYAWNRKRISNCVADYSVLYNSASFSTFKCYGVSPTNLXDLCFTNVXADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHXPNTXCGP

SEQ ID NO: 18 - protein, 195 aa, Artificial Sequence
  Recombinant polypeptide
  MISC_FEATURE (56)..(56): Xaa is Ser or Thr
  MISC_FEATURE (64)..(64): Xaa is Ser or Thr
  MISC_FEATURE (188)..(188): Xaa is Ser or Thr
  MISC_FEATURE (192)..(192): Xaa is Ser or Thr
TNLCPFGEVFNATRFASVYAWNRKNISNCVADYSVLYNSASFSTFKCYNVSPTNLXDLCFTNVXADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHXPNTXCGP

SEQ ID NO: 19 - protein, 195 aa, Artificial Sequence
  Recombinant polypeptide
  MISC_FEATURE (56)..(56): Xaa is Ser or Thr
  MISC_FEATURE (64)..(64): Xaa is Ser or Thr
  MISC_FEATURE (188)..(188): Xaa is Ser or Thr
  MISC_FEATURE (192)..(192): Xaa is Ser or Thr
TNLCPFGEVFNATRFASVYAWNRKNISNCVADYSVLYNSASFSTFKCYGVSPTNLXDLCFTNVXADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHXPNTXCGP

SEQ ID NO: 20 - protein, 195 aa, Artificial Sequence
  Recombinant polypeptide
  MISC_FEATURE (64)..(64): Xaa is Ser or Thr
  MISC_FEATURE (188)..(188): Xaa is Ser or Thr
TNLCPFGEVFNATRFASVYAWNRKNISNCVADYSVLYNSASFSTFKCYNVSPTKLNDLCFTNVXADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHXPATVCGP

SEQ ID NO: 21 - protein, 195 aa, Artificial Sequence
  Recombinant polypeptide
  MISC_FEATURE (64)..(64): Xaa is Ser or Thr
TNLCPFGEVFNATRFASVYAWNRKNISNCVADYSVLYNSASFSTFKCYNVSPTKLNDLCFTNVXADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 22 - protein, 195 aa, Artificial Sequence
  Recombinant polypeptide
  MISC_FEATURE (30)..(30): Xaa is Ser or Thr
  MISC_FEATURE (40)..(40): Xaa is Ser or Thr
  MISC_FEATURE (56)..(56): Xaa is Ser or Thr
  MISC_FEATURE (64)..(64): Xaa is Ser or Thr
  MISC_FEATURE (130)..(130): Xaa is Ser or Thr
  MISC_FEATURE (151)..(151): Xaa is Ser or Thr
  MISC_FEATURE (173)..(173): Xaa is Ser or Thr
  MISC_FEATURE (188)..(188): Xaa is Ser or Thr
  MISC_FEATURE (192)..(192): Xaa is Ser or Thr
TNLCPFGEVFNATRFASVYAWNRKNISNCXADYSVLYNSXSFSTFKCYNVSPTNLXDLCFTNVXADSFVIRGDEVRQIAPGQTGKIADYNYKLPDNFTGCVIAWNSNNLDSKVGGNYNYLYRLFRKSNLXPFERDISTEIYQAGSTPCNGXEGFNCYFPLQSYGFQPTNGNGXQPYRVVVLSFELNHXPNTXCGP

SEQ ID NO: 23 - protein, 14 aa, Artificial Sequence
  Recombinant polypeptide
QYIKANSKFIGITE

SEQ ID NO: 24 - protein, 12 aa, Artificial Sequence
  Recombinant polypeptide
NILMQYIKANSK

SEQ ID NO: 25 - protein, 18 aa, Artificial Sequence
  Recombinant polypeptide
NILMQYIKANSKFIGITE

SEQ ID NO: 26 - protein, 16 aa, Artificial Sequence
  Recombinant polypeptide
LKFIIKRYTPNNEIDS

SEQ ID NO: 27 - protein, 11 aa, Artificial Sequence
  Recombinant polypeptide
LYNGLKFIIKR

SEQ ID NO: 28 - protein, 20 aa, Artificial Sequence
  Recombinant polypeptide
LYNGLKFIIKRYTPNNEIDS

SEQ ID NO: 29 - protein, 14 aa, Artificial Sequence
  Recombinant polypeptide
KTTAALSILPGIGS

SEQ ID NO: 30 - protein, 15 aa, Artificial Sequence
  Recombinant polypeptide
FFLLTRILTIPQSLD

SEQ ID NO: 31 - protein, 19 aa, Artificial Sequence
  Recombinant polypeptide
EYLNKIQNSLSTEWSCSVT

SEQ ID NO: 32 - protein, 195 aa, Artificial Sequence [sequence omitted]

SEQ ID NO: 33 - protein, 197 aa, Artificial Sequence
  Recombinant polypeptide
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 34 - protein, 12 aa, Artificial Sequence
  Recombinant polypeptide
GGGSGGGSGGGS

SEQ ID NO: 35 - protein, 6 aa, Artificial Sequence
  Recombinant polypeptide
HGKHGK

SEQ ID NO: 36 - protein, 13 aa, Artificial Sequence
  Recombinant polypeptide
AKFVAAWTLKAAA

SEQ ID NO: 37 - protein, 15 aa, Artificial Sequence
  Recombinant polypeptide
ATPHFDYIASEVSKG

SEQ ID NO: 38 - protein, 15 aa, Artificial Sequence
  Recombinant polypeptide
FGVITADTLEQAIER

SEQ ID NO: 39 - protein, 15 aa, Artificial Sequence
  Recombinant polypeptide
FDYIASEVSKGLADL

SEQ ID NO: 40 - protein, 19 aa, Artificial Sequence
  Recombinant polypeptide
ATPHFDYIASEVSKGLADL

SEQ ID NO: 41 - protein, 37 aa, Artificial Sequence
  Recombinant polypeptide
ATPHFDYIASEVSKGLADLGGSFGVITADTLEQAIER

SEQ ID NO: 42 - protein, 43 aa, Artificial Sequence
  Recombinant polypeptide
ATPHFDYIASEVSKGLADLSLELRKPITFGVITADTLEQAIER

SEQ ID NO: 43 - protein, 3 aa, Artificial Sequence
  Recombinant polypeptide
GGS

SEQ ID NO: 44 - protein, 4 aa, Artificial Sequence
  Recombinant polypeptide
GGGS

SEQ ID NO: 45 - protein, 6 aa, Artificial Sequence
  Recombinant polypeptide
GGSGGS

SEQ ID NO: 46 - protein, 9 aa, Artificial Sequence
  Recombinant polypeptide
GGSGGSGGS

SEQ ID NO: 47 - protein, 8 aa, Artificial Sequence
  Recombinant polypeptide
GGGSGGGS

SEQ ID NO: 48 - protein, 149 aa, Artificial Sequence
  Recombinant polypeptide
MVLILNGPNLNLLGRREPEVYGRTTLEELEALCEAWGAELGLGVVFRQTNYEGQLIEWVQNAWQEGFLAIVLNPGALTHYSYALLDAIRAQPLPVVEVHLTNLHAREEFRRHSVTAPAARGIVSGFGPLSYKLALVYLAETLEVGGEGF

SEQ ID NO: 49 - protein, 32 aa, Artificial Sequence
  Recombinant polypeptide
GGGSATPHFDYIASEVSKGLADLGGSGGSGGS

SEQ ID NO: 50 - protein, 50 aa, Artificial Sequence
  Recombinant polypeptide
GGGSATPHFDYIASEVSKGLADLGGSFGVITADTLEQAIERGGSGGSGGS

SEQ ID NO: 51 - protein, 1273 aa, Artificial Sequence; identical to SEQ ID NO: 11
  Recombinant polypeptide

SEQ ID NO: 52 - protein, 151 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGF

SEQ ID NO: 53 - protein, 199 aa, Artificial Sequence
  Recombinant polypeptide
FPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 54 - protein, 199 aa, Artificial Sequence
  Recombinant polypeptide
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 55 - protein, 201 aa, Artificial Sequence
  Recombinant polypeptide
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 56 - protein, 203 aa, Artificial Sequence
  Recombinant polypeptide
FPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 57 - protein, 236 aa, Artificial Sequence
  Recombinant polypeptide
TNLCPFGEVFNATRFASVYAWNRKNISNCVADYSVLYNSASFSTFKCYGVSPTNLTDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGGSAKFVAAWTLKAAAGGSKSSIASFFFIIGLIIGLFLVLR

SEQ ID NO: 58 - protein, 267 aa, Artificial Sequence
  Recombinant polypeptide
MGILPSPGMPALLSLVSLLSVLLMGCVAETGTNLCPFGEVFNATRFASVYAWNRKNISNCVADYSVLYNSASFSTFKCYGVSPTNLTDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGGSAKFVAAWTLKAAAGGSKSSIASFFFIIGLIIGLFLVLR

SEQ ID NO: 59 - protein, 235 aa, Artificial Sequence
  Recombinant polypeptide
TNLCPFGEVFNATRFASVYAWNRKNISNCVADYSVLYNSASFSTFKCYGVSPTNLTDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGGSGGSGGSGGSGGSGGSKSSIASFFFIIGLIIGLFLVLR

SEQ ID NO: 60 - protein, 266 aa, Artificial Sequence
  Recombinant polypeptide
MGILPSPGMPALLSLVSLLSVLLMGCVAETGTNLCPFGEVFNATRFASVYAWNRKNISNCVADYSVLYNSASFSTFKCYGVSPTNLTDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGGSGGSGGSGGSGGSGGSKSSIASFFFIIGLIIGLFLVLR

SEQ ID NO: 61 - protein, 248 aa, Artificial Sequence
  Recombinant polypeptide
TNLCPFGEVFNATRFASVYAWNRKNISNCVADYSVLYNSASFSTFKCYGVSPTNLTDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGGSGGSGGSAKFVAAWTLKAAAGGSGGSGGSKSSIASFFFIIGLIIGLFLVLR

SEQ ID NO: 62 - protein, 279 aa, Artificial Sequence
  Recombinant polypeptide
MGILPSPGMPALLSLVSLLSVLLMGCVAETGTNLCPFGEVFNATRFASVYAWNRKNISNCVADYSVLYNSASFSTFKCYGVSPTNLTDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGGSGGSGGSAKFVAAWTLKAAAGGSGGSGGSKSSIASFFFIIGLIIGLFLVLR

SEQ ID NO: 63 - protein, 254 aa, Artificial Sequence
  Recombinant polypeptide
TNLCPFGEVFNATRFASVYAWNRKNISNCVADYSVLYNSASFSTFKCYGVSPTNLTDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGGSGGSGGSATPHFDYIASEVSKGLADLGGSGGSGGSKSSIASFFFIIGLIIGLFLVLR

SEQ ID NO: 64 - protein, 285 aa, Artificial Sequence
  Recombinant polypeptide
MGILPSPGMPALLSLVSLLSVLLMGCVAETGTNLCPFGEVFNATRFASVYAWNRKNISNCVADYSVLYNSASFSTFKCYGVSPTNLTDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGGSGGSGGSATPHFDYIASEVSKGLADLGGSGGSGGSKSSIASFFFIIGLIIGLFLVLR

SEQ ID NO: 65 - protein, 272 aa, Artificial Sequence
  Recombinant polypeptide
TNLCPFGEVFNATRFASVYAWNRKNISNCVADYSVLYNSASFSTFKCYGVSPTNLTDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGGSGGSGGSATPHFDYIASEVSKGLADLGGSFGVITADTLEQAIERGGSGGSGGSKSSIASFFFIIGLIIGLFLVLR

SEQ ID NO: 66 - protein, 303 aa, Artificial Sequence
  Recombinant polypeptide
MGILPSPGMPALLSLVSLLSVLLMGCVAETGTNLCPFGEVFNATRFASVYAWNRKNISNCVADYSVLYNSASFSTFKCYGVSPTNLTDLCFTNVSADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGGSGGSGGSATPHFDYIASEVSKGLADLGGSFGVITADTLEQAIERGGSGGSGGSKSSIASFFFIIGLIIGLFLVLR

SEQ ID NO: 67 - protein, 272 aa, Artificial Sequence
  Recombinant polypeptide
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGSGGSGGSATPHFDYIASEVSKGLADLGGSFGVITADTLEQAIERGGSGGSGGSKSSIASFFFIIGLIIGLFLVLR

SEQ ID NO: 68 - protein, 303 aa, Artificial Sequence
  Recombinant polypeptide
MGILPSPGMPALLSLVSLLSVLLMGCVAETGTNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGSGGSGGSATPHFDYIASEVSKGLADLGGSFGVITADTLEQAIERGGSGGSGGSKSSIASFFFIIGLIIGLFLVLR

SEQ ID NO: 69 - protein, 272 aa, Artificial Sequence
  Recombinant polypeptide
TNLCPFGEVFNATRFASVYAWNRKNITNCVADYSVLYNSASFSTFKCYGVSPTNLTDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGGSGGSGGSATPHFDYIASEVSKGLADLGGSFGVITADTLEQAIERGGSGGSGGSKSSIASFFFIIGLIIGLFLVLR

SEQ ID NO: 70 - protein, 303 aa, Artificial Sequence
  Recombinant polypeptide
MGILPSPGMPALLSLVSLLSVLLMGCVAETGTNLCPFGEVFNATRFASVYAWNRKNITNCVADYSVLYNSASFSTFKCYGVSPTNLTDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGGSGGSGGSATPHFDYIASEVSKGLADLGGSFGVITADTLEQAIERGGSGGSGGSKSSIASFFFIIGLIIGLFLVLR

SEQ ID NO: 71 - protein, 272 aa, Artificial Sequence
  Recombinant polypeptide
TNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGGSGGSGGSATPHFDYIASEVSKGLADLGGSFGVITADTLEQAIERGGSGGSGGSKSSIASFFFIIGLIIGLFLVLR

SEQ ID NO: 72 - protein, 303 aa, Artificial Sequence
  Recombinant polypeptide
MGILPSPGMPALLSLVSLLSVLLMGCVAETGTNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGGSGGSGGSATPHFDYIASEVSKGLADLGGSFGVITADTLEQAIERGGSGGSGGSKSSIASFFFIIGLIIGLFLVLR

SEQ ID NO: 73 - protein, 389 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGTKHGKHGK

SEQ ID NO: 74 - protein, 407 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGTKHHHHHH

SEQ ID NO: 75 - protein, 389 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGTKHHHHHH

SEQ ID NO: 76 - protein, 358 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGTNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGTKGSGSGS

SEQ ID NO: 77 - protein, 358 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGTNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGTKKKKKKK

SEQ ID NO: 78 - protein, 358 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGTNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGTKHGKHGK

SEQ ID NO: 79 - protein, 358 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGTNLCPFGEVFNATRFASVYAWNRKNITNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHHHHHH

SEQ ID NO: 80 - protein, 358 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGTNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHHHHHH

SEQ ID NO: 81 - protein, 358 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGTNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 82 - protein, 360 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 83 - protein, 389 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 84 - protein, 407 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 85 - protein, 360 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 86 - protein, 389 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 87 - protein, 407 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 88 - protein, 360 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 89 - protein, 389 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 90 - protein, 407 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 91 - protein, 360 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 92 - protein, 389 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 93 - protein, 407 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 94 - protein, 360 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 95 - protein, 389 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 96 - protein, 407 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 97 - protein, 360 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 98 - protein, 389 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 99 - protein, 407 aa, Artificial Sequence
  Recombinant polypeptide
NGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 100 - protein, 569 aa, Artificial Sequence
  Recombinant polypeptide
NITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 101 - protein, 598 aa, Artificial Sequence
  Recombinant polypeptide
NITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 102 - protein, 616 aa, Artificial Sequence
  Recombinant polypeptide
NITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 103 - protein, 598 aa, Artificial Sequence
  Recombinant polypeptide
NITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 104 - protein, 627 aa, Artificial Sequence
  Recombinant polypeptide
NITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 105 - protein, 645 aa, Artificial Sequence
  Recombinant polypeptide
NITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 106 - protein, 616 aa, Artificial Sequence
  Recombinant polypeptide
NITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 107 - protein, 645 aa, Artificial Sequence
  Recombinant polypeptide
NITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 108 - protein, 663 aa, Artificial Sequence
  Recombinant polypeptide
NITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTNLTDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 109 - protein, 569 aa, Artificial Sequence
  Recombinant polypeptide
NITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 110 - protein, 598 aa, Artificial Sequence
  Recombinant polypeptide
NITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 111 - protein, 616 aa, Artificial Sequence
  Recombinant polypeptide
NITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 112 - protein, 598 aa, Artificial Sequence
  Recombinant polypeptide
NITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 113 - protein, 627 aa, Artificial Sequence
  Recombinant polypeptide
NITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 114 - protein, 645 aa, Artificial Sequence
  Recombinant polypeptide
NITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 115 - protein, 616 aa, Artificial Sequence
  Recombinant polypeptide
NITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 116 - protein, 645 aa, Artificial Sequence
  Recombinant polypeptide
NITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 117 - protein, 663 aa, Artificial Sequence
  Recombinant polypeptide
NITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATNFSSVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 118 - protein, 569 aa, Artificial Sequence
  Recombinant polypeptide
NITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 119 - protein, 598 aa, Artificial Sequence
  Recombinant polypeptide
NITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 120 - protein, 616 aa, Artificial Sequence
  Recombinant polypeptide
NITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 121 - protein, 598 aa, Artificial Sequence
  Recombinant polypeptide
NITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 122 - protein, 627 aa, Artificial Sequence
  Recombinant polypeptide
NITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 123 - protein, 645 aa, Artificial Sequence
  Recombinant polypeptide
NITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 124 - protein, 616 aa, Artificial Sequence
  Recombinant polypeptide
NITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 125 - protein, 645 aa, Artificial Sequence
  Recombinant polypeptide
NITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 126 - protein, 663 aa, Artificial Sequence
  Recombinant polypeptide
NITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATNFSSVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 127 - protein, 569 aa, Artificial Sequence
  Recombinant polypeptide
NITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 128 - protein, 598 aa, Artificial Sequence
  Recombinant polypeptide
NITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 129 - protein, 616 aa, Artificial Sequence
  Recombinant polypeptide
NITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 130 - protein, 598 aa, Artificial Sequence
  Recombinant polypeptide
NITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 131 - protein, 627 aa, Artificial Sequence
  Recombinant polypeptide
NITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 132 - protein, 645 aa, Artificial Sequence
  Recombinant polypeptide
NITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 133 - protein, 616 aa, Artificial Sequence
  Recombinant polypeptide
NITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 134 - protein, 645 aa, Artificial Sequence
  Recombinant polypeptide
NITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 135 - protein, 663 aa, Artificial Sequence
  Recombinant polypeptide
NITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 136 - protein, 569 aa, Artificial Sequence
  Recombinant polypeptide
NITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 137 - protein, 598 aa, Artificial Sequence
  Recombinant polypeptide
NITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 138 - protein, 616 aa, Artificial Sequence
  Recombinant polypeptide
NITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 139 - protein, 598 aa, Artificial Sequence
  Recombinant polypeptide
NITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 140 - protein, 627 aa, Artificial Sequence
  Recombinant polypeptide
NITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 141 - protein, 645 aa, Artificial Sequence
  Recombinant polypeptide
NITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 142 - protein, 616 aa, Artificial Sequence
  Recombinant polypeptide
NITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 143 - protein, 645 aa, Artificial Sequence
  Recombinant polypeptide
NITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 144 - protein, 663 aa, Artificial Sequence
  Recombinant polypeptide
NITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTNLTDLCFTNVYADSFVIRGDEVRQIAPGQTGKIADYNYKLPDNFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 145 - protein, 569 aa, Artificial Sequence
  Recombinant polypeptide
NITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 146 - protein, 598 aa, Artificial Sequence
  Recombinant polypeptide
NITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 147 - protein, 616 aa, Artificial Sequence
  Recombinant polypeptide
NITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGTGNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 148 - protein, 598 aa, Artificial Sequence
  Recombinant polypeptide
NITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 149 - protein, 627 aa, Artificial Sequence
  Recombinant polypeptide
NITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 150 - protein, 645 aa, Artificial Sequence
  Recombinant polypeptide
NITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 151 - protein, 616 aa, Artificial Sequence
  Recombinant polypeptide
NITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGTGNITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 152 - protein, 645 aa, Artificial Sequence
  Recombinant polypeptide
NITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSGGSGGSNITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 153 - protein, 663 aa, Artificial Sequence
  Recombinant polypeptide
NITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGKGGGSATPHFDYIASEVSKGLADLGGSFGVITADTLEQAIERGGSGGSGGSNGSVLILNGPNLNLLGRREPEVYGNTTLEELNASAEAWGAELGLGVVFNQTNYEGQLIEWVQNASQEGFLAIVLNPGALTHYSYALLDAIRAQPLPVVEVHLTNLHAREEFRRHSVTAPAARGIVSGFGPLSYKLALVYLAETLEVGGEGFGGGSATPHFDYIASEVSKGLADLGGSFGVITADTLEQAIERGGSGGSGGSNITNLCPFGEVFNATRFASVYAWNRKNITNCVADYSVLYNSASFSTFKCYGVSPTKLNDLCFTNVSADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELNHTPATVCGPGTKHGKHGK

SEQ ID NO: 154 - protein, 221 aa, Artificial sequence
  Recombinant polypeptide
ETGNITNLCPFSEVFNATTFASVYAWNRKRISNCVADYSVLYNSTSFSTFQCYGVSSTKLNDLCFTNVYADSFVVRGDEVRQIAPGQTGVIADYNYKLPDDFTGCVLAWNSRNQDASTSGNFNYYYRIWRSEKLRPFERDIAHYDYQVGTQFKSSLKNYGFYSSAGDSHQPYRVVVLSFELLNAPATVCGPGTKHHHHHHGGSGGSGLNDIFEAQKIEWHE

SEQ ID NO: 155 - protein, 211 aa, Artificial sequence
  Recombinant polypeptide
ETGNITNVCPFHKVFNATRFPSVYAWERTKISDCIADYTVFYNSTSFSTFKCYGVSPSKLIDLCFTSVYADTFLIRFSEVRQVAPGQTGVIADYNYKLPDDFTGCVIAWNTAKQDVGNYFYRSHRSTKLKPFERDLSSDENGVRTLSTYDFNPNVPLEYQATRVVVLSFELLNAPATVCGPGTKHHHHHHGGSGGSGLNDIFEAQKIEWHE

SEQ ID NO: 156 - protein, 211 aa, Artificial sequence
  Recombinant polypeptide
ETGNITNFCPFDKVFNATRFPNVYAWQRTKISDCIADYTVLYNSTSFSTFKCYGVSPSKLIDLCFTSVYADTFLIRFSEVRQIAPGETGVIADYNYKLPDDFTGCVLAWNTAKQDIGSYFYRSHRAVKLKPFERDLSSDENGVRTLSTYDFNPNVPLDYQATRVVVLSFELLNAPATVCGPGTKHHHHHHGGSGGSGLNDIFEAQKIEWHE

(19) United States
(12) Patent Application Publication
(10) Pub. No.: US 2024/0131148 A1
(43) Pub. Date: Apr. 25, 2024

(54) IMMUNOGENIC COMPOSITIONS

(71) Applicant: THE SCRIPPS RESEARCH INSTITUTE, La Jolla, CA (US)

(72) Inventors: William R. SCHIEF, La Jolla, CA (US); Torben SCHIFFNER, La Jolla, CA (US); Jon M. STEICHEN, La Jolla, CA (US); Xiaozhen HU, La Jolla, CA (US)

(21) Appl. No.: 18/257,808

(22) PCT Filed: Dec. 17, 2021
(86) PCT No.: PCT/US2021/064100

§ 371 Date: Jun. 14, 2023

Related U.S. Application Data
(60) Provisional application No. 63/127,966, filed on Dec. 18, 2020.

(51) Int. Cl. A61K 39/215; A61K 39/00; C07K 14/005; C12N 7/00; C12N 9/88

(52) U.S. Cl. CPC A61K 39/215; C07K 14/005; C12N 7/00; C12N 9/88; A61K 2039/53; C12N 2740/16034; C12N 2760/20034; C12N 2770/20034; C12Y 402/0101

(57) ABSTRACT

Provided herein are glycan engineered SARS-CoV-2 RBD polypeptides, fusion polypeptides comprising thereof, and immunogenic compositions comprising thereof. Also provided are methods of administering the RBD polypeptide, fusion polypeptide or immunogenic composition to a subject to elicit an immune response. Also provided are polynucleotides encoding the fusion polypeptide, and methods of administering a composition comprising the polynucleotide to a subject to elicit an immune response. In some embodiments, the polynucleotide is an RNA comprising modified ribonucleotides.

### GOVERNMENT INTEREST

This invention was made with government support under grant number AI144462 awarded by the National Institutes of Health. The government has certain rights in the invention.

### FIELD OF THE INVENTION

The field of the invention generally relates to glycan engineered SARS-CoV-2 RBD polypeptides, fusion polypeptides comprising and polynucleotides encoding thereof, immunogenic compositions comprising thereof, and methods of using the immunogenic compositions in eliciting an immune response.

### CROSS-REFERENCE TO RELATED APPLICATIONS

This application claims the benefit of U.S. Application No. 63/127,966, filed Dec. 18, 2020, which is incorporated herein by reference in its entirety.

### BACKGROUND

Coronaviruses (CoVs) have been responsible for several outbreaks over the past two decades, including SARS-CoV in 2002-2003, MERS-CoV in 2012 (de Wit E et al. Nat Rev Microbiol. 2016; 14:523-34), and the current COVID-19 pandemic, caused by SARS-CoV-2, which began in late 2019 (Tse L V et al. Frontiers in microbiology. 2020; 11:658).

COVID-19 has emerged as a global public health crisis, joining severe acute respiratory syndrome (SARS) and Middle East respiratory syndrome (MERS) in a growing number of coronavirus-associated illnesses which have jumped from animals to people. There are at least seven identified coronaviruses that infect humans. SARS-CoV-2 was isolated and sequenced from human airway epithelial cells from infected patients (Zhu, N. et al. N. Engl. J. Med. 382, 727-733 (2020) and Wu, F. et al. Nature 579, 265-269 (2020)). Disease symptoms range from mild flu-like to severe cases with life-threatening pneumonia (Huang, C. et al. Lancet 395, 497-506 (2020)). The global situation is dynamically evolving, and on 30 Jan. 2020 the World Health Organization declared COVID-19 as a public health emergency of international concern (PHEIC), and on Mar. 11, 2020 it was declared a global pandemic.

Infections have spread to multiple continents. Human-to-human transmission has been observed in multiple countries, and a shortage of disposable personal protective equipment, and prolonged survival times of coronaviruses on inanimate surfaces, have compounded this already delicate situation and heightened the risk of nosocomial infections. The scale of the COVID-19 pandemic has led to unprecedented efforts by the research community to rapidly identify and test therapeutics and vaccines, and to understand the molecular basis of SARS-CoV-2 entry, pathogenesis, and immune targeting.

A sub-region of the SARS-CoV-2 Spike protein possesses a receptor-binding region (RBD) and there is high homology between SARS-CoV-2 and SARS-CoV. Structural studies have identified multiple conformational B cell epitopes and mapped binding of the RBD to the ACE2 receptor (Wrapp et al., Science 367, 1260-1263 (2020) 13 Mar. 2020; Walls et al., Cell 180, 281-292, Apr. 16, 2020). The RBD is predicted to possess B cell (Ser438-Gln506, Thr553-Glu583, Gly404-Aps427, Thr345-Ala352, and Lys529-Lys535) and T cell (9 CD4 and 11 CD8 T cell antigenic determinants) epitopes. (see e.g., Su Q D, et al., The biological characteristics of SARS-CoV-2 spike protein Pro330-Leu650. Vaccine. 2020 Apr. 30:50264-410X(20)30587-9. doi: 10.1016/j.vaccine.2020.04.070).

Several recent papers describe neutralizing (nAbs) and non-neutralizing anti-SARS-CoV-2 antibodies from convalescent donors. Several studies isolate nAbs (range of potencies, from highly potent to very weak) and non-nAbs, from convalescent donors. The majority of potent nAbs bind RBD and compete with ACE2. The most potent nAbs compete with ACE2 and protect against challenge in animal models. (T. F. Rogers et al., Science 10.1126/science.abc7520 (2020), P. J. M. Brouwer et al., Science 10.1126/science.abc5902 (2020), A. Z. Wec et al., Science 10.1126/science.abc7424 (2020), J. Hansen et al., Science 10.1126/science.abd0827 (2020), Wu et al., Science 368, 1274-1278 (2020), Ju, B. et al. Nature https://doi.org/10.1038/s41586-020-2380-z (2020), Seydoux et al. https://doi.org/10.1101/2020.05.12.091298doi: bioRxiv preprint, and Robbiani et al. https://doi.org/10.1101/2020.05.13.092619doi: bioRxiv preprint). Seydoux et al. indicates that the most potent nAb is called CV30 and the structure is reported by Hurlburt et al. (https://doi.org/10.1101/2020.06.12.148692doi: bioRxiv preprint). The structure of CV30 shows that the nAb binds to RBD with the epitope overlapping that of ACE2.

Structural studies of spike and/or RBD with or without antibodies or ACE2 have also been performed. Yan et al. (Science 367, 1444-1448 (2020)) reports the structure of ACE2 binding to RBD. Walls et al (2020, Cell 180, 281-292) reports the structure and antigenicity of the SARS-CoV-2 spike protein (the entire trimer). Wrapp et al (Science 367, 1260-1263 (2020)) reports the structure and antigenicity of the SARS-CoV-2 spike protein (the entire trimer).

The main target for nAbs on coronaviruses is the spike (S) protein that is anchored in the viral membrane. While epitopes capable of eliciting neutralizing antibodies must exist, the epitopes may be hidden and/or be insufficiently immunodominant to reliably induce a neutralizing antibody response. The S protein comprises two subdomains, the N-terminal S1 domain, which contains the N-terminal domain (NTD) and the receptor-binding domain (RBD) and the S2 domain. Upon receptor binding and membrane fusion, the S protein undergoes a conformational change from a prefusion state to a postfusion state compatible with merging of viral and target cell membranes. While most nAb epitopes may be presented on the prefusion conformation, when expressed as recombinant proteins, S proteins have a propensity to switch to the postfusion state.

In the SARS-CoV, MERS-CoV and SARS-CoV-2 outbreaks, neutralizing antibodies (nAbs) obtained from plasma of recovered patients have been used to decrease viral load and reduce mortality. Instead of polyclonal mixtures, an alternative strategy would be to administer purified monoclonal antibodies with neutralizing capacity. There is a continued need to identify and produce such neutralizing antibodies.

Furthermore, effective control of the SARS-CoV-2 outbreak depends on the development of effective vaccines against the virus. Thus, there is a need for a vaccine that reproducibly elicits a neutralizing antibody response in human subjects.

### BRIEF SUMMARY

In one aspect, provided herein are non-naturally occurring pathogen surface glycoprotein receptor binding domains (RBD) comprising an engineered glycosylation site. In some embodiments, the pathogen is a coronavirus. In some embodiments, the engineered glycosylation site comprises substitution of N at the position to be glycosylated or substitution of S or T at the position two amino acids towards the C-terminus from an existing N of the surface glycoprotein RBD, so as to create the motif N-X-S/T, so long as X is not proline. In some embodiments, the engineered glycosylation site is at one or more of amino acid positions 357, 381, 386, 394, and 428 according to the amino acid numbering of the SARS-CoV-2 S glycoprotein (e.g., SEQ ID NO:51).

In one aspect, provided herein are fusion polypeptides comprising (a) at least one viral polypeptide comprising a SARS-CoV spike protein (S), a SARS-CoV-2 spike protein (S), or an immunogenic fragment thereof; and (b) an amino acid sequence that targets the fusion polypeptide to the cell surface or a self-assembling domain capable of forming a nanoparticle. In some embodiments, the fusion polypeptide comprises a SARS-CoV-2 spike protein (S) or an immunogenic fragment thereof. In some embodiments, the fusion polypeptide comprises a receptor binding domain (RBD) of the SARS-CoV-2 spike protein (e.g., SEQ ID NO:51).

In some embodiments, the fusion polypeptide comprises a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein.

In some embodiments, a fusion polypeptide described herein further comprises an amino acid sequence that targets the fusion polypeptide to the cell surface. In some embodiments, the amino acid sequence that targets the fusion polypeptide to the cell surface comprises a GPI anchor signal sequence. In some embodiments, the amino acid sequence that targets the fusion polypeptide to the cell surface comprises a transmembrane domain.

In some embodiments, a fusion polypeptide described herein further comprises a self-assembling domain capable of forming a nanoparticle. In some embodiments, the self-assembling domain comprises a type II 3-Dehydroquinase, ferritin or lumazine synthase. In some embodiments, the self-assembling domain comprises a type II 3-Dehydroquinase polypeptide comprising one or more engineered glycosylation site. In some embodiments, the self-assembling domain comprises a Thermus thermophilus type type II 3-Dehydroquinase, optionally comprising one or more engineered glycosylation site.

In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40), ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42).

In some embodiments, a fusion polypeptide described herein further comprises a signal peptide.

In one aspect, provided herein are isolated polynucleotides encoding a fusion polypeptide described herein. In some embodiments, the polynucleotide is RNA. In some embodiments, the polynucleotide is mRNA comprising modified ribonucleotides.

In one aspect, provided herein are vectors comprising a polynucleotide described herein.

In one aspect, provided herein are host cells comprising a polynucleotide described herein.

In one aspect, provided herein are recombinant viruses comprising a polynucleotide described herein.

In one aspect, provided herein are immunogenic compositions comprising a fusion polypeptide described herein, a polynucleotide described herein, a vector described herein, or a recombinant virus described herein. In some embodiments, the immunogenic composition comprises a fusion polypeptide described herein. In some embodiments, the immunogenic composition comprises a polynucleotide encoding a fusion polypeptide described herein. In some embodiments, the polynucleotide is DNA. In some embodiments, the polynucleotide is mRNA, e.g., mRNA comprising modified nucleotides. In some embodiments, the immunogenic composition further comprises an adjuvant.

In one aspect, provided herein are pharmaceutical compositions comprising a fusion polypeptide described herein, a polynucleotide described herein, a vector described herein, a recombinant virus described herein, or an immunogenic composition described herein and a pharmaceutically acceptable excipient. In some embodiments, the pharmaceutical composition comprises a fusion polypeptide described herein. In some embodiments, the pharmaceutical composition comprises a polynucleotide encoding a fusion polypeptide described herein. In some embodiments, the polynucleotide is DNA. In some embodiments, the polynucleotide is mRNA, e.g., mRNA comprising modified nucleotides.

In one aspect, provided herein are methods of vaccinating a subject comprising administering to a subject a therapeutically effective amount of a fusion polypeptide described herein, a polynucleotide described herein, a vector described herein, a recombinant virus described herein, an immunogenic composition described herein or a pharmaceutical composition described herein to the subject. In some embodiments, the method of vaccinating comprises administering a fusion polypeptide described herein. In some embodiments, the method of vaccinating comprises administering a polynucleotide encoding a fusion polypeptide described herein. In some embodiments, the polynucleotide is DNA. In some embodiments, the polynucleotide is mRNA, e.g., mRNA comprising modified nucleotides. In some embodiments, the subject is a human.

In one aspect, provided herein are methods of inducing an immune response in a subject comprising administering an effective amount of a fusion polypeptide described herein, a polynucleotide described herein, a vector described herein, a recombinant virus described herein, an immunogenic composition described herein or a pharmaceutical composition described herein to the subject. In some embodiments, the method of inducing an immune response comprises administering a fusion polypeptide described herein. In some embodiments, the method of inducing an immune response comprises administering a polynucleotide encoding a fusion polypeptide described herein. In some embodiments, the polynucleotide is DNA. In some embodiments, the polynucleotide is mRNA, e.g., mRNA comprising modified nucleotides. In some embodiments, the immune response produces neutralizing antibodies against SARS-CoV-2, e.g., neutralizing antibodies against the receptor binding domain (RBD) of SARS-CoV-2. In some embodiments, the subject is a human.

In one aspect, provided herein are methods of treating a viral infection in a subject comprising administering a therapeutically effective amount of a fusion polypeptide described herein, a polynucleotide described herein, a vector described herein, a recombinant virus described herein, an immunogenic composition described herein or a pharmaceutical composition described herein to the subject. In some embodiments, the viral infection is a SARS-CoV-2 infection. In some embodiments, the viral infection is COVID-19. In some embodiments, the method of treating a viral infection comprises administering a fusion polypeptide described herein. In some embodiments, the method of treating a viral infection comprises administering a polynucleotide encoding a fusion polypeptide described herein. In some embodiments, the polynucleotide is DNA. In some embodiments, the polynucleotide is mRNA, e.g., mRNA comprising modified nucleotides. In some embodiments, the subject is a human.

In one aspect, provided herein are methods of preventing or reducing the likelihood of a viral infection in a subject comprising administering a therapeutically effective amount of a fusion polypeptide described herein, a polynucleotide described herein, a vector described herein, a recombinant virus described herein, an immunogenic composition described herein or a pharmaceutical composition described herein to the subject. In some embodiments, the viral infection is a SARS-CoV-2 infection. In some embodiments, the viral infection is COVID-19. In some embodiments, the method of preventing or reducing the likelihood of a viral infection comprises administering a fusion polypeptide described herein. In some embodiments, the method of preventing or reducing the likelihood of a viral infection comprises administering a polynucleotide encoding a fusion polypeptide described herein. In some embodiments, the polynucleotide is DNA. In some embodiments, the polynucleotide is mRNA, e.g., mRNA comprising modified nucleotides. In some embodiments, the subject is a human.

In one aspect, provided herein are methods of producing the fusion polypeptide described herein. In some embodiments, the method comprises culturing a host cell described herein under suitable conditions to produce the fusion polypeptide.

In one aspect, provided herein are methods of producing a polynucleotide encoding a fusion polypeptide described herein. In some embodiments, the polynucleotide comprises RNA, e.g., mRNA and is produced through in vitro transcription. In some embodiments, the polynucleotide comprises RNA, e.g., mRNA and is produced through chemical synthesis.

### BRIEF DESCRIPTION OF THE DRAWINGS

FIG. 1. Low dose human CD4 T-cell responses to individual lumazine synthase peptides. 15-mer level CD4 T-cell responses are shown. Each dot represents the CD4 T-cell response to that peptide from a given subject. Each color indicates a unique subject. One subject was excluded from analysis due to high background in the unstimulated control.

FIG. 2. Low dose cytokine+ human CD4 T-cell responses to lumanzine synthase. Positive CD4 T-cell responses by Fisher's exact test are shown. Data displayed is restricted to positive responses. Percent of positive responders for the top 3 peptides is indicated on the graph. Each dot represents the CD4 T-cell response to that peptide from a given subject. Each color indicates a unique subject.

FIG. 3. Protein structural model showing the glycosylation sites added to the SARS-CoV-2 receptor binding domain (RBD). SARS-CoV-2 S trimer is gray; one of the three RBD domains is shown in the center in the “up” conformation; and a single ACE2 is shown in the upper left corner. Spheres on the RBD indicate glycosylation sites that were added to memRBD_v175 or are other potential sites for glycan masking.

FIG. 4. Schematic illustrating the design layout for memRBD variants.

FIG. 5. Cell surface antigenicity of memRBD variants with different linker regions.

FIG. 6. Antigenic profile of memRBD variants with different glycan-masking.

FIG. 7. Schematic illustrating the design layout for RBD-12mer nanoparticles.

FIG. 8. Expression yield and assembly and homogeneity of RBD-12mers. A. Yields were determined by OD₂₈₀ measurement. B. Preparative SEC purification chromatograms. C. SEC-MALS data. D: Negative stain electron tomography of RBD-12mer-1.

FIG. 9. Bio-Layer Interferometry (BLI) analysis of antigenicity of RBD-12mer-1 and RBD-12mer-2. A: Comparison of monovalent binding affinities of SARS-CoV-2-specific Fabs binding to RBD monomer, RBD-12mers, and stabilized SARS-CoV-2 S protein trimer (2P). B: Comparison of binding avidities of SARS-CoV-2-specific IgG antibodies binding to RBD monomer, RBD-12mers, and stabilized SARS-CoV-2 S protein trimer (2P). NB: No binding.

FIG. 10. Comparing antigenicity of different RBD-12mers by Bio-Layer Interferometry (BLI).

FIG. 11. SARS-CoV-2 RBD-NP 24mer immunization.

### DETAILED DESCRIPTION

In one aspect, provided herein are fusion polypeptides comprising glycol engineered and/or membrane-tethered or nanoparticle-tethered SARS-CoV-2 receptor binding domain (RBD) constructs (e.g., memRBD and RBD-12mer).

In some embodiments, the glycol engineered RBD constructs described herein are effective to elicit an immune response against SARS-CoV-2.

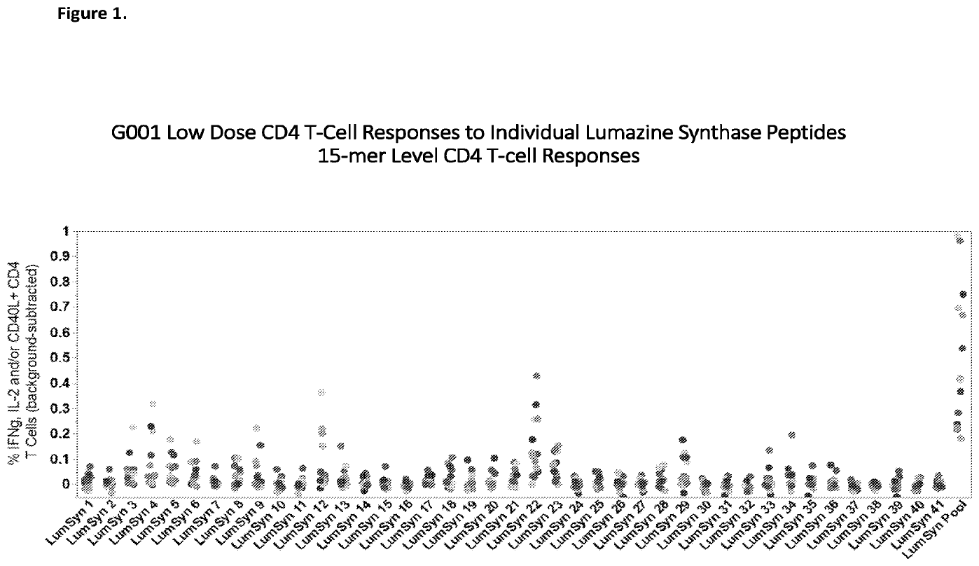

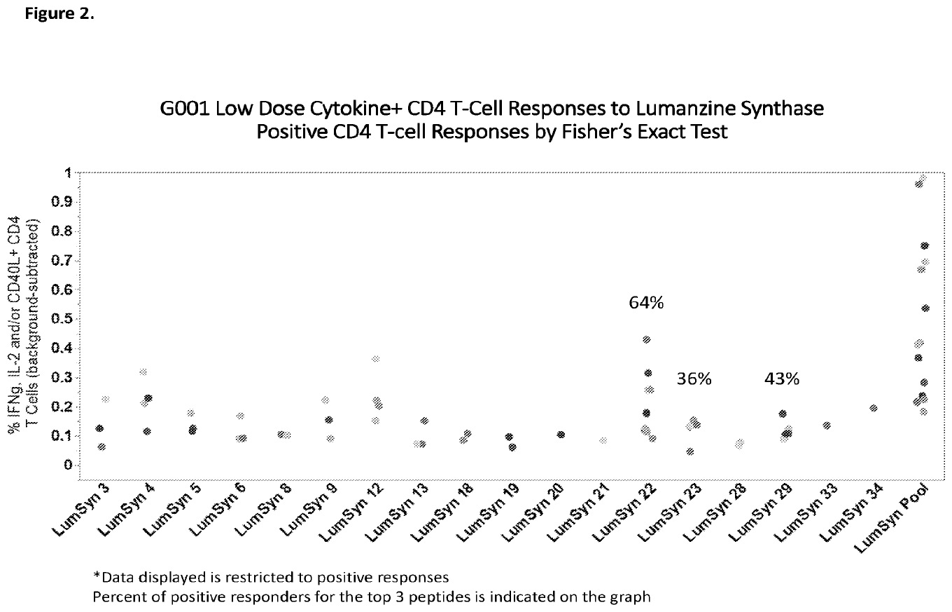

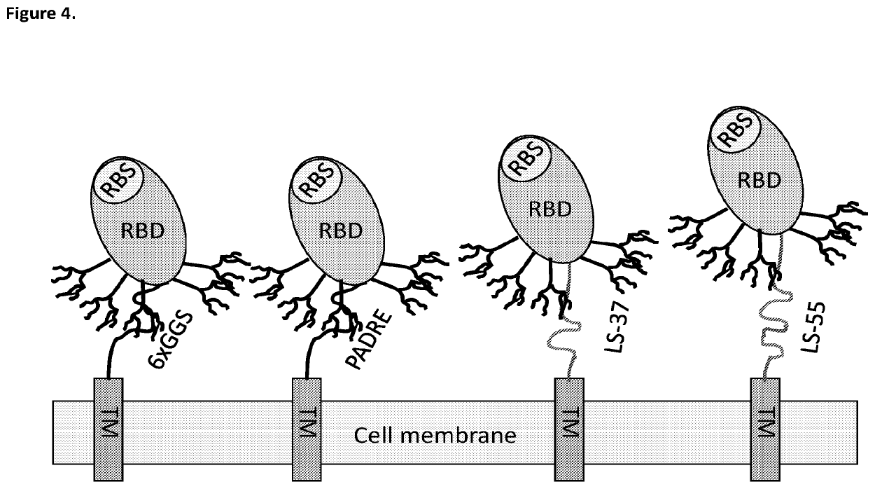

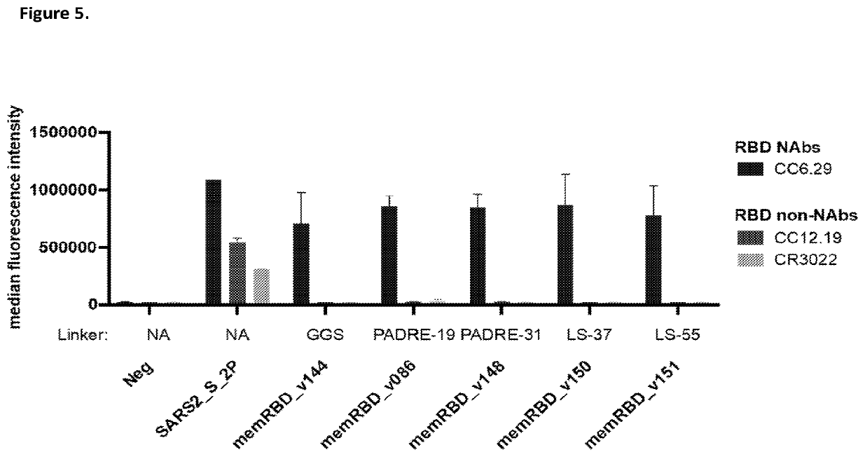

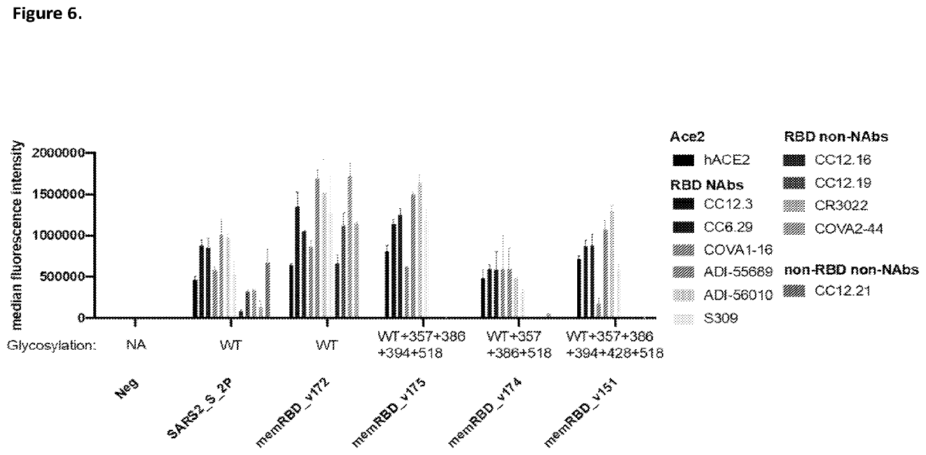

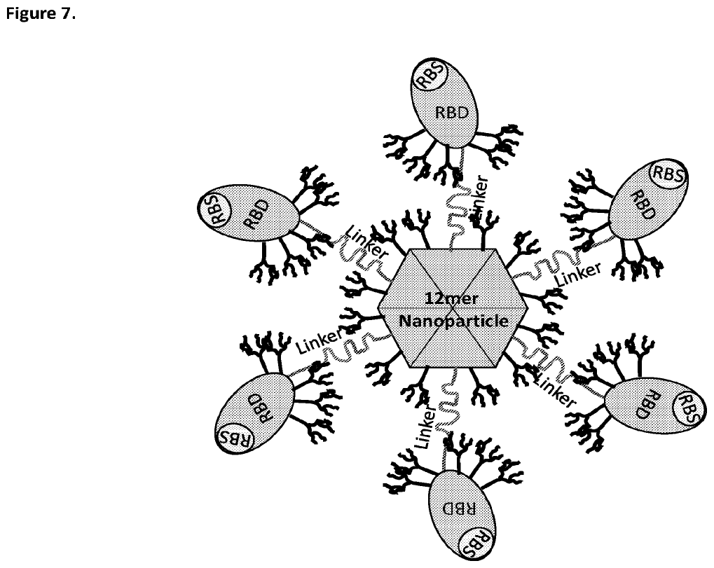

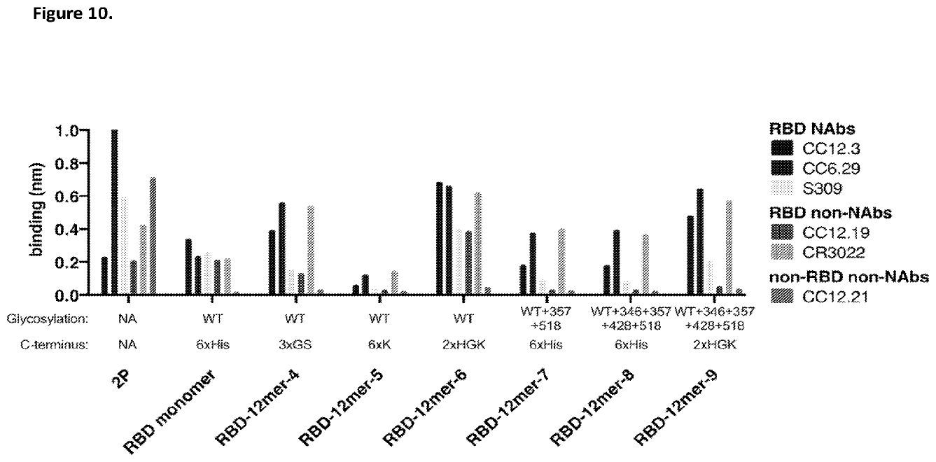

In some embodiments, the immunogens described herein aim to elicit potent neutralizing antibodies against the receptor binding domain (RBD) of SARS-CoV-2. In some embodiments, the immunogens are based on the RBD tethered to a transmembrane domain via a flexible linker (FIG. 4). In some embodiments, the immunogens are based on the RBD tethered to a self-assembling polypeptide capable of forming a nanoparticle via a flexible linker (FIG. 7). In some embodiments, glycosylation sites have been engineered into the RBD in order to mask the portion of the RBD surface that would be occluded on the SARS-CoV-2 spike trimer (FIGS. 5, 6, 9 and 10). Antibodies targeting surfaces occluded on the trimer should be non-neutralizing. Without being bound by a particular theory, the engineered glycosylation sites prevent binding or elicitation of non-neutralizing antibodies. The added glycans block binding of non-neutralizing or weak-neutralizing RBD antibodies but do not hinder binding of potent neutralizing RBD antibodies (FIGS. 6 and 10), thus these fusion constructs should be able to elicit a focused, potently neutralizing response. The focused response should allow for protective responses from lower vaccine doses, reducing the cost of each dose and increasing the number of people that can be vaccinated from one batch of vaccine. In some embodiments, the tether includes an MHC class II T cell epitope (e.g., an epitope described herein or the universal Pan DR epitope (PADRE) CD4 T cell epitope), which increases B cell responses in diverse humans (FIGS. 1 and 2). In some embodiments, delivery of such constructs is by nucleic acid or viral vector approaches. The small size of the memRBD and RBD-12mer constructs compared to the full-length spike protein provides other advantages: it contributes to dose sparing for nucleic acid delivery, and, in the context of viral vector delivery the smaller size of the insert reduces the burden on viral fitness.

In some embodiments, the fusion polypeptides described herein elicit focused neutralizing antibody responses, in contrast to the full length spike, which induces both neutralizing antibody and non-neutralizing antibody responses. This allows for dose sparing and avoid problems associated with non-neutralizing antibody elicitation. In some embodiments, the cell-surface expression level of a membrane tethered fusion polypeptide described herein is higher than for the full length spike, which also allows for dose sparing. In some embodiments, a fusion polypeptide described herein is smaller than the full length spike, thus further allowing for dose sparing in the case of nucleic acid delivery and for easier incorporation into viral vectors.

In one aspect, provided herein are SARS antigens designed to promote induction of nAbs against SARS-CoV-2. The S protein of SARS-CoV-2 and SARS-CoV show considerable structural and sequence homology. In some embodiments, the SARS antigens described herein promote induction of nAbs against SARS-CoV and related viruses.

In one aspect, provided herein are additional glycans and refined glycan positioning, enhanced CD4 T help, including on the intracellular side of the TM domain, multimerization to increase B cell activation and alternate transmembrane domains for further improved expression.

### Definitions

Unless defined otherwise, all technical and scientific terms used herein have the same meaning as commonly understood by one of ordinary skill in the art to which this disclosure is related. To facilitate an understanding of the described methods, a number of terms and phrases are defined below.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides described herein are based upon antibodies, in certain embodiments, the polypeptides can occur as single chains or associated chains.

A “fragment” is a portion of a protein or nucleic acid that is substantially identical to a reference protein or nucleic acid. In some embodiments, the portion retains at least 50%, 75%, or 80%, or 90%, 95%, or even 99% of the biological activity of the reference protein or nucleic acid described herein.

The term “immune response” includes T cell mediated and/or B cell mediated immune responses that are influenced by modulation of T cell costimulation. Exemplary immune responses include T cell responses, e.g., cytokine production. In addition, the term immune response includes immune responses that are indirectly affected by T cell activation, e.g., antibody production (humoral responses) and activation of cytokine responsive cells, e.g., macrophages.

The term “MHC class II T cell epitope” refers to a peptide sequence which can be bound by class II MHC molecules in the form of a peptide-presenting MHC molecule or MHC complex and then, in this form, be recognized and bound by CD4 T-helper cells.

An “antigen” is a molecule capable of stimulating an immune response, and can be produced by infectious agents or cancer cells or an autoimmune disease. Antigens recognized by T cells, whether helper T lymphocytes (T helper (TH) cells) or cytotoxic T lymphocytes (CTLs), are not recognized as intact proteins, but rather as small peptides in association with HLA class I or class II proteins on the surface of cells. During the course of a naturally occurring immune response, antigens that are recognized in association with HLA class II molecules on antigen presenting cells (APCs) are acquired from outside the cell, internalized, and processed into small peptides that associate with the HLA class II molecules. APCs can also cross-present peptide antigens by processing exogenous antigens and presenting the processed antigens on HLA class I molecules. Antigens that give rise to peptides that are recognized in association with HLA class I MHC molecules are generally peptides that are produced within the cells, and these antigens are processed and associated with class I MHC molecules. It is now understood that the peptides that associate with given HLA class I or class II molecules are characterized as having a common binding motif, and the binding motifs for a large number of different HLA class I and II molecules have been determined. Synthetic peptides that correspond to the amino acid sequence of a given antigen and that contain a binding motif for a given HLA class I or II molecule can also be synthesized. These peptides can then be added to appropriate APCs, and the APCs can be used to stimulate a T helper cell or CTL response either in vitro or in vivo. Methods for synthesizing the peptides, and methods for stimulating a T helper cell or CTL response are all known and readily available to one of ordinary skill in the art.

The terms “linker,” “spacer,” and “hinge” are used interchangeably herein to refer to a peptide or other chemical linkage located between two or more otherwise independent functional domains of an immunogenic composition. For example, a linker may be located between an immunogenic polypeptide and a target antigen. In some embodiments, the linker is a polypeptide located between two domains of a fusion polypeptide, e.g., an immunogenic polypeptide and a target antigen. Suitable linkers for coupling the two or more domains are described herein and/or will otherwise be clear to a person skilled in the art.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin et al, Proc. Natl. Acad. Sci., 87:2264-2268 (1990), as modified in Karlin et al., Proc. Natl. Acad. Sci., 90:5873-5877 (1993), and incorporated into the NBLAST and XBLAST programs (Altschul et al., Nucleic Acids Res., 25:3389-3402 (1991)). In certain embodiments, Gapped BLAST can be used as described in Altschul et al., Nucleic Acids Res. 25:3389-3402 (1997). BLAST-2, WU-BLAST-2 (Altschul et al., Methods in Enzymology, 266:460-480 (1996)), ALIGN, ALIGN-2 (Genentech, South San Francisco, California) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48):444-453 (1970)) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, WI 53711). Bestfit uses the local homology algorithm of Smith and Waterman (Advances in Applied Mathematics 2: 482 489 (1981)) to find the best segment of homology between two sequences. When using Bestfit or any other sequence alignment program to determine whether a particular sequence is, for instance, 95% identical to a reference sequence described herein, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides described herein are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. Identity can exist over a region of the sequences that is at least about 10, about 20, about 40-60 residues in length or any integral value there between, and can be over a longer region than 60-80 residues, for example, at least about 90-100 residues, and in some embodiments, the sequences are substantially identical over the full length of the sequences being compared, such as the coding region of a nucleotide sequence for example.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In some embodiments, conservative substitutions in the sequences of the polypeptides and antibodies described herein do not abrogate the binding of the polypeptide or antibody containing the amino acid sequence, to the antigen(s). Methods of identifying nucleotide and amino acid conservative substitutions which do not eliminate antigen binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:.412-417 (1997)).

As used herein, the terms “treatment” or “therapy” (as well as different forms thereof, including curative or palliative) refer to treatment of an infected person. As used herein, the term “treating” includes alleviating or reducing at least one adverse or negative effect or symptom of a condition, disease or disorder. In some embodiments, the condition, disease or disorder is COVID-19. In some embodiments, the condition, disease or disorder is a cancer or tumor.

Terms such as “treating” or “treatment” or “to treat” or “alleviating” or “to alleviate” refer to therapeutic measures that cure, slow down, lessen symptoms of, and/or halt progression of a diagnosed pathologic condition or disorder, such as a viral infection. Thus, those in need of treatment include those already diagnosed with or suspected of having the disorder. In certain embodiments, a subject is successfully “treated” for the disorder according to the methods described herein if the patient shows one or more of the following: a reduction in the number of or complete absence of viral load; a reduction in the viral burden; inhibition of or an absence of the virus into peripheral organs; relief of one or more symptoms associated with the disorder; reduced morbidity and mortality; improvement in quality of life, or any combination thereof. In some embodiments, the pathologic condition or disorder is infection with SARS-CoV-2. In some embodiments, the pathologic condition or disorder is COVID-19.

As used herein, the terms “prevention” or “prophylaxis” refer to preventing a subject from becoming infected with, or reducing the risk of a subject from becoming infected with, or halting transmission of, or the reducing the risk of transmission of a pathogen, e.g., a virus, bacteria, or parasite. In some embodiments, the pathogen is a virus. In some embodiments, the pathogen is SARS-CoV-2. Prophylactic or preventative measures refer to measures that prevent and/or slow the development of a targeted pathological condition or disorder. Thus, those in need of prophylactic or preventative measures include those prone to have the disorder and those in whom the disorder is to be prevented.

As employed above and throughout the disclosure the term “effective amount” refers to an amount effective, at dosages, and for periods of time necessary, to achieve the desired result, for example, with respect to the treatment of the relevant disorder, condition, or side effect. An “effective amount” can be determined empirically and in a routine manner, in relation to the stated purpose. It will be appreciated that the effective amount of components described herein will vary from subject to subject not only with the particular vaccine, component or composition selected, the route of administration, and the ability of the components to elicit a desired result in the individual, but also with factors such as the disease state or severity of the condition to be alleviated, hormone levels, age, sex, weight of the individual, the state of being of the subject, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide the improved therapeutic response. An effective amount is also one in which any toxic or detrimental effects of the components are outweighed by the therapeutically beneficial effects.

The term “therapeutically effective amount” refers to an amount of a polypeptide, polynucleotide, recombinant virus, immunogenic composition, therapeutic composition, or other drug effective to “treat” a disease or disorder in a subject or mammal. A “prophylactically effective amount” refers to an amount effective, at dosages and for periods of time necessary, to achieve the desired prophylactic result.

The terms “subject,” “individual,” and “patient” are used interchangeably herein, and refer to an animal, for example a human, to whom treatment, including prophylactic treatment, with a immunogenic composition or pharmaceutical composition described herein, is provided. In some embodiments, a subject is a human. In some embodiments, the subject is a non-human animal, for example, a mouse or a cynomolgus monkey. In some embodiments, the subject is a swine, cattle, sheep, goat or rabbit. In some embodiments, the subject is a mink. In some embodiments, the subject is a chicken or turkey.

In one embodiment, the subject, individual, or patient has been infected with a pathogen, e.g., a virus, bacteria or parasite. In one embodiment, the subject, individual, or patient suffers from an infection, e.g., a viral, bacterial or parasitic infection. In one embodiment, the subject, individual, or patient has been exposed to a pathogen, e.g., a virus, bacteria or parasite. In one embodiment, the subject, individual, or patient is at risk of being exposed to a pathogen, e.g., a virus, bacteria or parasite. In one embodiment, the subject, individual, or patient has been infected with a virus, e.g., SARS-CoV-2. In one embodiment, the subject, individual, or patient suffers from a viral infection, e.g., COVID-19. In one embodiment, the subject, individual, or patient has been exposed to a virus, e.g., SARS-CoV-2. In one embodiment, the subject, individual, or patient is at risk of being exposed to a virus, e.g., SARS-CoV-2. In some embodiments, the subject, individual, or patient has a cancer or tumor. In some embodiments, the cancer or tumor is melanoma or glioblastoma. In some embodiments, the cancer or tumor is lung cancer, non-small cell lung cancer, renal cancer, breast cancer, pancreatic cancer, nasopharyngeal cancer, ovarian cancer, cervical cancer, sarcoma, colorectal cancer, HPV16 Associated Cervical Cancer, gastric cancer, or prostate cancer.

The terms “pharmaceutical composition,” “pharmaceutical formulation,” “pharmaceutically acceptable formulation,” or “pharmaceutically acceptable composition” all of which are used interchangeably, refer to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. “Pharmaceutically acceptable” or “pharmaceutical formulation” refers to a preparation which is in such form as to permit the biological activity of the active ingredient to be effective, and which contains no additional components which are unacceptably toxic to a subject to which the formulation would be administered. The formulation can be sterile.

In some embodiments, the term “about” refers to ranges of approximately 10-20% greater than or less than the indicated number or range. In further embodiments, “about” refers to plus or minus 10% of the indicated number or range. For example, “about 10%” indicates a range of 9% to 11%.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where embodiments of the disclosure are described in terms of a Markush group or other grouping of alternatives, the described composition or method encompasses not only the entire group listed as a whole, but also each member of the group individually and all possible subgroups of the main group, and also the main group absent one or more of the group members. The described compositions and methods also envisage the explicit exclusion of one or more of any of the group members in the described compositions and methods.

### Pathogen Surface Glycoprotein Receptor Binding Domains

In one aspect, provided herein are non-naturally occurring pathogen surface glycoprotein receptor binding domains (RBD) comprising an engineered glycosylation site. In some embodiments, the pathogen is a coronavirus. In some embodiments, the pathogen is SARS-CoV-2.

In some embodiments, the invention provides a non-naturally occurring pathogen surface glycoprotein RBD wherein the engineered glycosylation site comprises substitution of N at the position to be glycosylated or substitution of S or T at the position two amino acids towards the C-terminus from an existing N of the surface glycoprotein RBD, so as to create the motif N-X-S/T, so long as X is not proline.

N-linked glycosylation involves attachment of a carbohydrate consisting of several sugar molecules, sometimes also referred to as glycan, to the amide nitrogen of an asparagine (Asn) residue of a protein. This type of linkage is important for both the structure and function of many eukaryotic proteins. The N-linked glycosylation process occurs in eukaryotes and widely in archaea, but very rarely in bacteria. The nature of N-linked glycans attached to a glycoprotein is determined by the protein, and the cell in which it is expressed, and varies across species. The carbohydrate consists of sugar moieties, linked to one another in via glycosidic bonds. Attachment of a glycan residue to a protein requires the presence of the consensus sequence Asn-X-Ser/Thr wherein X is any amino acid except proline (Pro). Different species synthesize different types of N-linked glycan.

In some embodiments, provided herein is a non-naturally occurring coronavirus surface glycoprotein receptor binding domain (RBD) which comprises an engineered glycosylation site at one or more of amino acid positions 357, 381, 386, 394, and 428 according to the amino acid numbering of the SARS-CoV-2-S surface glycoprotein. In some embodiments, the RBD comprises a sequence that is at least 70%, at least 80%, at least 85%, at least 90%, at least 95%, at least 96%, at least 97%, at least 98%, or at least 99%, identical to the RBD of the SARS-CoV-2-S surface glycoprotein. In some embodiments, the RBD comprises the RBD of the SARS-CoV-2-S surface glycoprotein. In some embodiments, the RBD comprises a sequence that is at least 70%, at least 80%, at least 85%, at least 90%, at least 95%, at least 96%, at least 97%, at least 98%, or at least 99%, identical to the RBD of the SARS-CoV-S surface glycoprotein. In some embodiments, the RBD comprises the RBD of the SARS-CoV-S surface glycoprotein. (SARS-CoV-2-S understood to be on the surface of COVID-19; SARS-CoV-S understood to be on the surface of the 2002 SARS virus.) An engineered glycosylation site comprises substitution of “N” at the position intended to be glycosylated or substitution of S or T two amino acids towards the C-terminal from a pre-existing “N” intended to be glycosylated. The consensus sequence is Asn-X-Ser/Thr where X is any amino acid except proline. In some embodiments, the RBD comprises an engineered glycosylation site at position 357, or at position 381, or at position 386, or at position 394, or at position 428, or at positions 357 and 381, or at positions 357 and 386, or at positions 357 and 394, or at positions 357 and 428, or at positions 381 and 386, or at positions 381 and 394, or at positions 381 and 428, or at positions 386 and 394, or at positions 386 and 428, or at positions 394 and 428, or at positions 357, 381, and 386, or at positions 357, 381, and 394, or at positions 357, 381, and 428, or at positions 357, 386, and 394, or at positions 357, 386, and 428, or at positions 357, 394 and 428, or at positions 381, 386, and 394, or at positions 381, 386 and 428, or at positions 381, 394 and 428, or at positions 386, 394 and 428, or at positions 357, 381, 386, and 394, or at positions 357, 381, 386, and 428, or at positions 357, 381, 394 and 428, or at positions 357, 386, 394, and 428, or at positions 381, 386, 394, and 428, or at positions 357, 381, 386, 394, and 428. In some embodiments, provided herein is a coronavirus surface glycoprotein, which comprises a RBD of any aspect of this paragraph. In some embodiments, provided herein is a hybrid protein which comprises a RBD of any aspect of this paragraph. In some embodiments, provided herein is a hybrid protein which comprises the RBD operatively linked to a transmembrane domain and/or a secretion signal sequence. In some embodiments, the hybrid protein can have the RBD is operatively linked to the transmembrane domain by a linker or flexible linker, such as a G rich linker or flexible linker or a linker including a T cell epitope. In some embodiments, the T cell epitope comprises a PADRE CD4 T cell epitope. In some embodiments, the T cell epitope comprises an MHC class II T cell epitope comprising the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40).

In some embodiments, the RBD, or the pathogen or coronavirus surface glycoprotein or the hybrid protein is operatively linked to a T cell epitope, such as an MHC class II T cell epitope comprising the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the RBD, or the pathogen or coronavirus surface glycoprotein or the hybrid protein is operatively linked to a T cell epitope, such as the PADRE CD4 T cell epitope.

In some embodiments, the non-naturally occurring pathogen surface glycoprotein RBD comprises a sequence that is at least 70%, at least 80%, at least 85%, at least 90%, at least 95%, at least 96%, at least 97%, at least 98%, or at least 99%, identical to the RBD of the SARS-CoV-2-S surface glycoprotein; or comprising a sequence that is at least 70%, at least 80%, at least 85%, at least 90%, at least 95%, at least 96%, at least 97%, at least 98%, or at least 99%, identical to the RBD of the SARS-CoV-2-S glycoprotein; or comprising a sequence as set forth and/or exemplified herein and/or a sequences having at least 95%, 96%, 97%, 98%, or 99% identity to such a sequence as set forth and/or exemplified herein.

In some embodiments, the non-naturally occurring pathogen surface glycoprotein RBD comprises an engineered glycosylation site at one or more of amino acid positions 357, 381, 386, 394, and 428 according to the amino acid numbering of the SARS-CoV-2-S surface glycoprotein.

In some embodiments, the non-naturally occurring pathogen surface glycoprotein RBD comprises, with reference to the amino acid numbering of the SARS-CoV-2-S surface glycoprotein, an engineered glycosylation site at position 357, or at position 381, or at position 386, or at position 394, or at position 428, or at positions 357 and 381, or at positions 357 and 386, or at positions 357 and 394, or at positions 357 and 428, or at positions 381 and 386, or at positions 381 and 394, or at positions 381 and 428, or at positions 386 and 394, or at positions 386 and 428, or at positions 394 and 428, or at positions 357, 381, and 386, or at positions 357, 381, and 394, or at positions 357, 381, and 428, or at positions 357, 386, and 394, or at positions 357, 386, and 428, or at positions 357, 394 and 428, or at positions 381, 386, and 394, or at positions 381, 386 and 428, or at positions 381, 394 and 428, or at positions 386, 394 and 428, or at positions 357, 381, 386, and 394, or at positions 357, 381, 386, and 428, or at positions 357, 381, 394 and 428, or at positions 357, 386, 394, and 428, or at positions 381, 386, 394, and 428, or at positions 357, 381, 386, 394, and 428, or at positions 357, 381, 394, and 518.

In some embodiments, the non-naturally occurring pathogen surface glycoprotein RBD comprises with reference to the amino acid numbering of the SARS-CoV-2-S surface glycoprotein, an engineered N-glycosylation site at one or two or three or four or five or six or seven or eight of positions 357, 360, 381, 386, 394, 428, 518, and 522, in any combination. In some embodiments, the pathogen surface glycoprotein RBD comprises engineered N-glycosylation sites at positions 357, 381, 386, 394, and 428 (SEQ ID NO:13), or at positions 357, 394, 428, 518, and 522 (SEQ ID NO:14), or at positions 357, 394, 428, and 518 (SEQ ID NO:15), or at positions 357, 386, 394, 428, and 518 (SEQ ID NO:16), or at positions 386, 394, 518, and 522 (SEQ ID NO:17), or at positions 357, 381, 386, 394, 428, 518, and 522 (SEQ ID NO:18), or at positions 357, 386, 394, 428, 518, and 522 (SEQ ID NO:19), or at positions 357, 381, 394, and 428 (SEQ ID NO:21), or at positions 357, 381, 394, and 518 (SEQ ID NO:20). In some embodiments, the pathogen surface glycoprotein RBD comprises engineered N-glycosylation sites in a combination selected from Table 1.

In some embodiments, the pathogen surface glycoprotein RBDs described herein further comprise an N-glycosylation site at one or both of positions 460 and 481, or an N-glycosylation site at one or both of positions 370 and 386.

Other glycosylation sites may be added to mask additional parts of the RBD. The most potent SARS-CoV-2 neutralizing antibodies are directed to the ACE2-binding site, so additional glycans may be added to mask all except the ACE2-binding site.

The consensus sequence for N-glycosylation is Asn-Xaa-Ser/Thr wherein Xaa can be any amino acid except proline. Thus, one way to introduce an N-glycosylation site is by substituting in an Asn residue two amino acids N-terminal to a Ser or Thr residue. Another way to introduce an N-glycosylation site is by substituting in a Ser or Thr residue two amino acids downstream from a preexisting Asn residue. A third way to introduce an N-glycosylation site is by substituting an Asn residue at a desired location and a Ser or Thr residue two amino acids towards the C terminal of the protein.

In some embodiments, SARS-CoV-2 proteins described herein comprise N-glycosylation sites introduced at one or more of amino acid positions 357, 360, 370, 381, 386, 428, 460, 481, 503, 518, and 522 by substituting amino acid residues in the SARS-CoV-2 RBD (SEQ ID NO:9) as follows: aa 357: R357N; aa 360: V362S or V362T; aa 370: A372S or A372T; aa 381: G381N; aa 386: K386N with N388T or N388S; aa 394: Y396S or Y396T; aa 428: D428N; aa 460: K462S or K462T; aa 481: V483S or V483T; aa 503: V503N with Y505S or Y505T; aa 518: L518N with A520S or A520T; aa 522: A522N with V524S or V524T. Likewise, N-glycosylation sites can be added at corresponding positions of other coronaviruses, including but not limited to SARS-CoV, MERS-CoV, and mutants of SARS-CoV-2, SARS-CoV, and MERS-CoV as they exist or arise in the population from time to time.

In some embodiments, the coronavirus RBDs and surface glycoproteins described herein comprise, with reference to the amino acid numbering of the SARS-CoV-2-S surface glycoprotein, an engineered glycosylation site at position 357, or at position 381, or at position 386, or at position 394, or at position 428, or at positions 357 and 381, or at positions 357 and 386, or at positions 357 and 394, or at positions 357 and 428, or at positions 381 and 386, or at positions 381 and 394, or at positions 381 and 428, or at positions 386 and 394, or at positions 386 and 428, or at positions 394 and 428, or at positions 357, 381, and 386, or at positions 357, 381, and 394, or at positions 357, 381, and 428, or at positions 357, 386, and 394, or at positions 357, 386, and 428, or at positions 357, 394 and 428, or at positions 381, 386, and 394, or at positions 381, 386 and 428, or at positions 381, 394 and 428, or at positions 386, 394 and 428, or at positions 357, 381, 386, and 394, or at positions 357, 381, 386, and 428, or at positions 357, 381, 394 and 428, or at positions 357, 386, 394, and 428, or at positions 381, 386, 394, and 428, or at positions 357, 381, 386, 394, and 428.

In some embodiments, the coronavirus RBDs and surface glycoproteins described herein comprise, with reference to the amino acid numbering of the SARS-CoV-2-S surface glycoprotein, an engineered glycosylation site at an engineered N-glycosylation site at one or two or three or four or five or six or seven or eight of positions 357, 360, 381, 386, 394, 428, 518, and 522, in any combination. In some embodiments, the coronavirus RBDs and surface glycoproteins comprise an engineered N-glycosylation site at positions 357, 381, 386, 394, and 528, or at positions 357, 394, 428, 518, and 522, or at positions 357, 394, 428, and 518, or at positions 357, 386, 394, 428, and 518, or at positions 386, 394, 518, and 522, or at positions 357, 381, 386, 394, 428, 518, and 522, or at positions 357, 386, 394, 428, 518, and 522. In some embodiments, the coronavirus RBDs and surface glycoproteins comprise engineered N-glycosylation sites in a combination selected from Table 1.

In some embodiments, coronavirus RBDs and surface glycoproteins described herein comprise N-glycosylation site at one or both of positions 460 and 481, or an N-glycosylation site at one or both of positions 370 and 386.

In one aspect, provided herein is a non-naturally occurring pathogen surface glycoprotein RBD wherein the RBD includes a linker to a transmembrane domain of the pathogen or coronavirus surface glycoprotein for cell surface expression.

In one aspect, provided herein is a non-naturally occurring pathogen surface glycoprotein RBD wherein the linker comprises a glycine rich linker, or GGSGGSGGSGGSGGS (SEQ ID NO:3), or a T-cell epitope, or a PADRE CD4 T cell epitope.

In one aspect, provided herein is a non-naturally occurring pathogen surface glycoprotein comprising the non-naturally occurring pathogen surface glycoprotein RBD, or a non-naturally occurring coronavirus surface glycoprotein comprising the non-naturally occurring pathogen surface glycoprotein RBD described herein.

In one aspect, provided herein is a non-naturally occurring pathogen or coronavirus surface glycoprotein described herein including a secretion signal sequence.

In one aspect, provided herein is a non-naturally occurring pathogen or coronavirus surface glycoprotein described herein including a moiety capable of binding to a metal hydroxide adjuvant; a moiety capable of binding to a metal hydroxide adjuvant at or near comprising within 25 amino acids of the N- or C-terminus; a moiety capable of binding to a metal hydroxide adjuvant comprising phosphoserine; a moiety capable of binding to a metal hydroxide adjuvant at or near comprising within 25 amino acids of the N- or C-terminus comprising phosphoserine; a moiety capable of binding to a metal hydroxide adjuvant comprising cysteine; a moiety capable of binding to a metal hydroxide adjuvant at or near comprising within 25 amino acids of the N- or C-terminus comprising cysteine; or any of the foregoing wherein the metal hydroxide adjuvant comprises aluminum hydroxide or alum or sodium bis(2-methoxyethoxy)aluminum hydride; or any of the foregoing comprising phosphoserine that can couple with a cysteine.

The following table provides amino acid sequences of the basic components discussed above, including the SARS-CoV-2 RBD and surface glycoprotein amino acid sequences used for reference.

TABLE 1
Table of Sequences

| SEQ | | |
|---|---|---|
| ID | Protein or | |
| NO | Component | Amino Acid Sequence |
| 1 | memRBD_v058 | MGILPSPGMP ALLSLVSLLS VLLMGCVAET GTNLCPFGEV |
| | | FNATRFASVY AWNRKNISNC VADYSVLYNS ASFSTFKCYN |
| | | VSPTNLTDLC FTNVSADSFV IRGDEVRQIA PGQTGKIADY |
| | | NYKLPDNFTG CVIAWNSNNL DSKVGGNYNY LYRLFRKSNL |
| | | KPFERDISTE IYQAGSTPCN GVEGFNCYFP LQSYGFQPTN |
| | | GVGYQPYRVV VLSFELLHAP ATVCGPGGSG GSGGSGGSGG |
| | | SGGSKIFIMI VGGLIGLRIV FAVLSVIHRV R |
| 2 | memRBD_v059 | MGILPSPGMP ALLSLVSLLS VLLMGCVAET GTNLCPFGEV |
| | | FNATRFASVY AWNRKNISNC VADYSVLYNS ASFSTFKCYN |
| | | VSPTNLTDLC FTNVSADSFV IRGDEVRQIA PGQTGKIADY |
| | | NYKLPDNFTG CVIAWNSNNL DSKVGGNYNY LYRLFRKSNL |
| | | KPFERDISTE IYQAGSTPCN GVEGFNCYFP LQSYGFQPTN |
| | | GVGYQPYRVV VLSFELLHAP ATVCGPGGSA KFVAAWTLKA |
| | | AAGGSKIFIM IVGGLIGLRI VFAVLSVIHR VR |
| 3 | GGS linker | GGSGGSGGSG GSGGSGGS |
| 4 | PADRE linker | GGSAKFVAAW TLKAAAGGS |
| 5 | pHLsec | MGILPSPGMP ALLSLVSLLS VLLMGCVAET G |
| 6 | HIV env TM | KIFIMIVGGL IGLRIVFAVL SVIHRVR |
| 7 | SARS-CoV-2 | KWPWYIWLGF IAGLIAIVMV TIML |
| | TM | |
| 8 | VSV-G TM | KSSIASFFFI IGLIIGLFLV LR |
| 9 | SARS-CoV-2 | TNLCPFGEVF NATRFASVYA WNRKRISNCV ADYSVLYNSA |
| | RBD | SFSTFKCYGV SPTKLNDLCF TNVYADSFVI RGDEVRQIAP |
| | GenBank | GQTGKIADYN YKLPDDFTGC VIAWNSNNLD SKVGGNYNYL |
| | QHD43416.1 | YRLFRKSNLK PFERDISTEI YQAGSTPCNG VEGFNCYFPL |
| | | QSYGFQPTNG VGYQPYRVVV LSFELLHAPA TVCGP |
| 10 | SARS-CoV RBD | TNLCPFGEVF NATKFPSVYA WERKKISNCV ADYSVLYNST |
| | GenBank | FFSTFKCYGV SATKLNDLCF SNVYADSFVV KGDDVRQIAP |
| | AAP41037.1 | GQTGVIADYN YKLPDDFMGC VLAWNTRNID ATSTGNYNYK |
| | | YRYLRHGKLR PFERDISNVP FSPDGKPCTP PALNCYWPLN |
| | | DYGFYTTTGI GYQPYRVVVL SFELLNAPAT VCGP |
| 11 | SARS-CoV-2 | MFVFLVLLPL VSSQCVNLTT RTQLPPAYTN SFTRGVYYPD |
| | surface | KVFRSSVLHS TQDLFLPFFS NVTWFHAIHV SGTNGTKRFD |
| | glycoprotein | NPVLPFNDGV YFASTEKSNI IRGWIFGTTL DSKTQSLLIV |
| | GenBank | NNATNVVIKV CEFQFCNDPF LGVYYHKNNK SWMESEFRVY |
| | QHD43416.1 | SSANNCTFEY VSQPFLMDLE GKQGNFKNLR EFVFKNIDGY |
| | | FKIYSKHTPI NLVRDLPQGF SALEPLVDLP IGINITRFQT |
| | | LLALHRSYLT PGDSSSGWTA GAAAYYVGYL QPRTFLLKYN |
| | | ENGTITDAVD CALDPLSETK CTLKSFTVEK GIYQTSNFRV |
| | | QPTESIVRFP NITNLCPFGE VFNATRFASV YAWNRKRISN |
| | | CVADYSVLYN SASFSTFKCY GVSPTKLNDL CFTNVYADSF |
| | | VIRGDEVRQI APGQTGKIAD YNYKLPDDFT GCVIAWNSNN |
| | | LDSKVGGNYN YLYRLFRKSN LKPFERDIST EIYQAGSTPC |
| | | NGVEGFNCYF PLQSYGFQPT NGVGYQPYRV VVLSFELLHA |
| | | PATVCGPKKS TNLVKNKCVN FNFNGLTGTG VLTESNKKFL |
| | | PFQQFGRDIA DTTDAVRDPQ TLEILDITPC SFGGVSVITP |
| | | GTNTSNQVAV LYQDVNCTEV PVAIHADQLT PTWRVYSTGS |
| | | NVFQTRAGCL IGAEHVNNSY ECDIPIGAGI CASYQTQTNS |
| | | PRRARSVASQ SIIAYTMSLG AENSVAYSNN SIAIPTNFTI |
| | | SVTTEILPVS MTKTSVDCTM YICGDSTECS NLLLQYGSFC |
| | | TQLNRALTGI AVEQDKNTQE VFAQVKQIYK TPPIKDFGGF |
| | | NFSQILPDPS KPSKRSFIED LLFNKVTLAD AGFIKQYGDC |
| | | LGDIAARDLI CAQKFNGLTV LPPLLTDEMI AQYTSALLAG |
| | | TITSGWTFGA GAALQIPFAM QMAYRFNGIG VTQNVLYENQ |
| | | KLIANQFNSA IGKIQDSLSS TASALGKLQD VVNQNAQALN |
| | | TLVKQLSSNF GAISSVLNDI LSRLDKVEAE VQIDRLITGR |
| | | LQSLQTYVTQ QLIRAAEIRA SANLAATKMS ECVLGQSKRV |
| | | DFCGKGYHLM SFPQSAPHGV VFLHVTYVPA QEKNFTTAPA |
| | | ICHDGKAHFP REGVFVSNGT HWFVTQRNFY EPQIITTDNT |
| | | FVSGNCDVVI GIVNNTVYDP LQPELDSFKE ELDKYFKNHT |
| | | SPDVDLGDIS GINASVVNIQ KEIDRLNEVA KNLNESLIDL |
| | | QELGKYEQYI KWPWYIWLGF IAGLIAIVMV TIMLCCMTSC |
| | | CSCLKGCCSC GSCCKFDEDD SEPVLKGVKL HYT |
| 12 | SARS-CoV | MFIFLLFLTL TSGSDLDRCT TFDDVQAPNY TQHTSSMRGV |
| | surface | YYPDEIFRSD TLYLTQDLFL PFYSNVTGFH TINHTFGNPV |
| | glycoprotein | IPFKDGIYFA ATEKSNVVRG WVFGSTMNNK SQSVIIINNS |
| | GenBank | TNVVIRACNF ELCDNPFFAV SKPMGTQTHT MIFDNAFNCT |
| | AAP41037.1 | FEYISDAFSL DVSEKSGNFK HLREFVFKNK DGFLYVYKGY |
| | | QPIDVVRDLP SGFNTLKPIF KLPLGINITN FRAILTAFSP |
| | | AQDIWGTSAA AYFVGYLKPT TFMLKYDENG TITDAVDCSQ |
| | | NPLAELKCSV KSFEIDKGIY QTSNFRVVPS GDVVRFPNIT |
| | | NLCPFGEVFN ATKFPSVYAW ERKKISNCVA DYSVLYNSTF |
| | | FSTFKCYGVS ATKLNDLCFS NVYADSFVVK GDDVRQIAPG |
| | | QTGVIADYNY KLPDDFMGCV LAWNTRNIDA TSTGNYNYKY |
| | | RYLRHGKLRP FERDISNVPF SPDGKPCTPP ALNCYWPLND |
| | | YGFYTTTGIG YQPYRVVVLS FELLNAPATV CGPKLSTDLI |
| | | KNQCVNFNFN GLIGTGVLTP SSKRFQPFQQ FGRDVSDFTD |
| | | SVRDPKTSEI LDISPCAFGG VSVITPGTNA SSEVAVLYQD |
| | | VNCTDVSTAI HADQLTPAWR IYSTGNNVFQ TQAGCLIGAE |
| | | HVDTSYECDI PIGAGICASY HTVSLLRSTS QKSIVAYTMS |
| | | LGADSSIAYS NNTIAIPTNF SISITTEVMP VSMAKTSVDC |
| | | NMYICGDSTE CANLLLQYGS FCTQLNRALS GIAAEQDRNT |
| | | REVFAQVKOM YKTPTLKYFG GFNFSQILPD PLKPTKRSFI |
| | | EDLLFNKVIL ADAGFMKQYG ECLGDINARD LICAQKENGL |
| | | TVLPPLLIDD MIAAYTAALV SGTATAGWTF GAGAALQIPF |
| | | AMQMAYRFNG IGVTQNVLYE NOKQIANQFN KAISQIQESL |
| | | TTTSTALGKL QDVVNQNAQA LNTLVKQLSS NFGAISSVLN |
| | | DILSRLDKVE AEVQIDRLIT GRLQSLQTYV TQQLIRAAEI |
| | | RASANLAATK MSECVLGQSK RVDFCGKGYH LMSFPQAAPH |
| | | GVVFLHVTYV PSQERNFTTA PAICHEGKAY FPREGVFVFN |
| | | GTSWFITQRN FFSPQIITTD NTFVSGNCDV VIGIINNTVY |
| | | DPLQPELDSF KEELDKYFKN HTSPDVDLGD ISGINASVVN |
| | | IQKEIDRLNE VAKNLNESLI DLQELGKYEQ YIKWPWYVWL |
| | | GFIAGLIAIV MVTILLCCMT SCCSCLKGAC SCGSCCKFDE |
| | | DDSEPVLKGV KLHYT |
| 13 | N-glycosylation | TNLCPFGEVF NATRFASVYA WNRKNISNCV ADYSVLYNSA |
| | at 357, 381, 386, | SFSTFKCYNV SPINLXDLCF TNVSADSFVI RGDEVRQIAP |
| | 384, 428 | GQTGKIADYN YKLPDNFTGC VIAWNSNNLD SKVGGNYNYL |
| | | YRLFRKSNLK PFERDISTEI YQAGSTPCNG VEGFNCYFPL |
| | | QSYGFQPTNG VGYQPYRVVV LSFELLHAPA TVCGP |
| | | wherein X is S or T |
| 14 | N-glycosylation | TNLCPFGEVF NATRFASVYA WNRKNISNCV ADYSVLYNSA |
| | at 357, 394, 428, | SFSTFKCYGV SPTKLNDLCF TNVSADSFVI RGDEVRQIAP |
| | 518, 522 | GQTGKIADYN YKLPDNFTGC VIAWNSNNLD SKVGGNYNYL |
| | | YRLFRKSNLK PFERDISTEI YQAGSTPCNG VEGFNCYFPL |
| | | QSYGFQPTNG VGYQPYRVVV LSFELNHXPN TXCGP |
| | | wherein X is S or T |
| 15 | N-glycosylation | TNLCPFGEVF NATRFASVYA WNRKNISNCV ADYSVLYNSA |
| | at 357, 394, 428, | SFSTFKCYGV SPTKLNDLCF TNVXADSFVI RGDEVRQIAP |
| | 518 | GQTGKIADYN YKLPDNFTGC VIAWNSNNLD SKVGGNYNYL |
| | | YRLFRKSNLK PFERDISTEI YQAGSTPCNG VEGFNCYFPL |
| | | QSYGFQPTNG VGYQPYRVVV LSFELNHXPA TVCGP |
| | | wherein X is S or T |
| 16 | N-glycosylation | TNLCPFGEVF NATRFASVYA WNRKNISNCV ADYSVLYNSA |
| | at 357, 386, 394, | SFSTFKCYGV SPTNLXDLCF TNVXADSFVI RGDEVRQIAP |
| | 428, 518 | GQTGKIADYN YKLPDNFTGC VIAWNSNNLD SKVGGNYNYL |
| | | YRLFRKSNLK PFERDISTEI YQAGSTPCNG VEGFNCYFPL |
| | | QSYGFQPTNG VGYQPYRVVV LSFELNHXPA TVCGP |
| | | wherein X is S or T |
| 17 | N-glycosylation | TNLCPFGEVF NATRFASVYA WNRKRISNCV ADYSVLYNSA |
| | at 386, 394, 518, | SFSTFKCYGV SPTNLXDLCF TNVXADSFVI RGDEVRQIAP |
| | 522 | GQTGKIADYN YKLPDDFIGC VIAWNSNNLD SKVGGNYNYL |
| | | YRLFRKSNLK PFERDISTEI YQAGSTPCNG VEGFNCYFPL |
| | | QSYGFQPTNG VGYQPYRVVV LSFELNHXPN TXCGP |
| | | wherein X is S or T |
| 18 | N-glycosylation | TNLCPFGEVF NATRFASVYA WNRKNISNCV ADYSVLYNSA |
| | at 357, 381, 386, | SFSTFKCYNV SPTNLXDLCF TNVXADSFVI RGDEVRQIAP |
| | 394, 428, 518, | GQTGKIADYN YKLPDNFTGC VIAWNSNNLD SKVGGNYNYL |
| | 522 | YRLFRKSNLK PFERDISTEI YQAGSTPCNG VEGFNCYFPL |
| | | QSYGFQPTNG VGYQPYRVVV LSFELNHXPN TXCGP |
| | | wherein X is S or T |
| 19 | N-glycosylation | TNLCPFGEVF NATRFASVYA WNRKNISNCV ADYSVLYNSA |
| | at 357, 386, 394, | SFSTFKCYGV SPTNLXDLCF TNVXADSFVI RGDEVRQIAP |
| | 428, 518, 522 | GQTGKIADYN YKLPDNFTGC VIAWNSNNLD SKVGGNYNYL |
| | | YRLFRKSNLK PFERDISTEI YQAGSTPCNG VEGFNCYFPL |
| | | QSYGFQPTNG VGYQPYRVVV LSFELNHXPN TXCGP |
| | | wherein X is S or T |
| 20 | N-glycosylation | TNLCPFGEVF NATRFASVYA WNRKNISNCV ADYSVLYNSA |
| | at 357, 381, 394, | SFSTFKCYNV SPTKLNDLCF TNVXADSFVI RGDEVRQIAP |
| | 518 | GQTGKIADYN YKLPDDFTGC VIAWNSNNLD SKVGGNYNYL |
| | | YRLFRKSNLK PFERDISTEI YQAGSTPCNG VEGFNCYFPL |
| | | QSYGFQPTNG VGYQPYRVVV LSFELNHXPA TVCGP |
| | | wherein X is S or T |
| 21 | N-glycosylation | TNLCPFGEVF NATRFASVYA WNRKNISNCV ADYSVLYNSA |
| | at 357, 381, 394, | SFSTFKCYNV SPTKLNDLCF TNVXADSFVI RGDEVRQIAP |
| | 428 | GQTGKIADYN YKLPDNFTGC VIAWNSNNLD SKVGGNYNYL |
| | | YRLFRKSNLK PFERDISTEI YQAGSTPCNG VEGFNCYFPL |
| | | QSYGFQPTNG VGYQPYRVVV LSFELLHAPA TVCGP |
| | | wherein X is S or T |
| 22 | N-glycosylation | TNLCPFGEVF NATRFASVYA WNRKNISNCX ADYSVLYNSX |
| | at 357, 360, 370, | SFSTFKCYNV SPTNLXDLCF TNVXADSFVI RGDEVRQIAP |
| | 381, 386, 394, | GQTGKIADYN YKLPDNFTGC VIAWNSNNLD SKVGGNYNYL |
| | 428, 460, 481, | YRLFRKSNLX PFERDISTEI YQAGSTPCNG XEGFNCYFPL |
| | 503, 518, 522 | QSYGFQPTNG NGXQPYRVVV LSFELNHXPN TXCGP |
| | | wherein X is S or T |

In some embodiment, the non-naturally occurring protein comprises the amino acid sequence of: MGILPSPGMPALLSLVSLLSVLLMGCVAETGTNLCPFGEVFNATRFASVYAWNRKNISN CVADYSVLYNSASFSTFKCYNVSPTNLTDLCFTNVSADSFVIRGDEVRQIAPGQTGKIAD YNYKLPDNFTGCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGPGGSGGSGGSGGS GGSGGSKIFIMIVGGLIGLRIVFAVLSVIHRVR (SEQ ID NO: 1; mem_RBD_v058). SEQ ID NO:1 includes five engineered glycosylation sites, at positions N357, N381, N386, N394, N428, and it includes a GGS linker (SEQ ID NO:43) between the RBD and TM domains. This construct expresses well on cell surface and has excellent antigenic profile in which neutralizing antibodies bind to it but non-neutralizing or weakly-neutralizing antibodies show no detectable binding.

In some embodiment, the non-naturally occurring protein comprises the amino acid sequence of MGILPSPGMPALLSLVSLLSVLLMGCVAETGTNLCPFGEVFNATRFASVYAWNRKNISN CVADYSVLYNSASFSTFKCYNVSPTNLTDLCFTNVSADSFVIRGDEVRQIAPGQTGKIAD YNYKLPDNFTGCVIAWNSNNLDSKVGGNYNYLYRLFRKSNLKPFERDISTEIYQAGSTPC NGVEGFNCYFPLQSYGFQPTNGVGYQPYRVVVLSFELLHAPATVCGPGGSAKFVAAWT LKAAAGGSKIFIMIVGGLIGLRIVFAVLSVIHRVR (SEQ ID NO: 2; memRBD_v059). SEQ ID NO:2 includes five engineered glycosylation sites, at positions N357, N381, N386, N394, N428, and includes a PADRE linker (SEQ ID NO:4) between RBD and TM domains. SEQ ID NO: 2 is the same as SEQ ID NO: 1 except it uses a PADRE linker instead of a GGS linker. This construct also has an excellent antigenic profile.

A secretion signal sequence from the pHLsec vector (MGILPSPGMPALLSLVSLLSVLLMGCVAETG; SEQ ID NO: 5) is indicated above, but any secretion signal sequence may be contemplated. The leader sequence will be cleaved during expression/secretion and is not present in the final expressed protein product. The embodiments contained herein are not limited to this particular leader sequence as different leader sequences could be used to serve the same purpose.

The transmembrane domain (TM) from HIV Env of the BG505 isolate (KIFIMIVGGLIGLRIVFAVLSVIHRVR; SEQ ID NO:6), but any TM domain will suffice. As examples, other TM domains could include the TM from SARS-CoV-2 (KWPWYIWLGFIAGLIAIVMVTIML; SEQ ID NO: 7) or the TM from VSV-G (KSSIASFFFIIGLIIGLFLVLR; SEQ ID NO: 8).

In some embodiments, mutations can be added to the RBD to increase expression levels and/or increase thermal stability.

In one aspect, provided herein is a diagnostic serological probe. In some embodiments, one or more epitopes that bind to a non-nAb is masked, providing a probe capable of detecting one or more nAbs in serum or other antibody mixture. In some embodiments, one or more epitopes that bind to a nAb is masked, providing a probe capable of detecting one or more non-nAbs in serum or other polyclonal mixture.

### Fusion Polypeptides

In one aspect, provided herein are fusion polypeptides comprising (a) at least one viral polypeptide comprising a SARS-CoV spike protein (S), a SARS-CoV-2 spike protein (S), or an immunogenic fragment thereof; and (b) an amino acid sequence that targets the fusion polypeptide to the cell surface or a self-assembling domain capable of forming a nanoparticle. In some embodiments, the fusion polypeptide comprises a SARS-CoV-2 spike protein (S) or an immunogenic fragment thereof. In some embodiments, the fusion polypeptide comprises a receptor binding domain (RBD) of the SARS-CoV-2 spike protein (e.g., SEQ ID NO:51). In some embodiments, the fusion polypeptide comprises an SARS-CoV-2 spike glycoprotein receptor binding domain described herein. In some embodiments, the SARS-CoV-2 spike glycoprotein receptor binding domain described herein comprises one or more engineered glycosylation sites.

In some embodiments, the fusion polypeptide comprises a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein.

In some embodiments, the fusion polypeptide comprises a SARS-CoV-2 spike protein (S) or an immunogenic fragment thereof. In some embodiments, the SARS-CoV-2 spike protein (S) comprises an amino acid sequence having at least 70%, at least 80%, at least 85%, at least 90%, at least 95%, at least 97%, at least 98% at least 99% or at least 100% identity with MFVFLVLLPLVSSQCVNLTTRTQLPPAYTNSFTRGVYYPDKVFRSSVLHSTQDLFLPFFSN VTWFHAIHVSGTNGTKRFDNPVLPFNDGVYFASTEKSNIIRGWIFGTTLDSKTQSLLIVNN ATNVVIKVCEFQFCNDPFLGVYYHKNNKSWMESEFRVYSSANNCTFEYVSQPFLMDLE GKQGNFKNLREFVFKNIDGYFKIYSKHTPINLVRDLPQGFSALEPLVDLPIGINITRFQTLL ALHRSYLTPGDSSSGWTAGAAAYYVGYLQPRTFLLKYNENGTITDAVDCALDPLSETKC TLKSFTVEKGIYQTSNFRVQPTESIVRFPNITNLCPFGEVFNATRFASVYAWNRKRISNCV ADYSVLYNSASFSTFKCYGVSPTKLNDLCFTNVYADSFVIRGDEVRQIAPGQTGKIADYN YKLPDDFTGCVIAWNSNNLDSKVGGNYNYLYRLFRKSNLKPFERDISTEIYQAGSTPCNG VEGFNCYFPLQSYGFQPTNGVGYQPYRVVVLSFELLHAPATVCGPKKSTNLVKNKCVNF NFNGLTGTGVLTESNKKFLPFQQFGRDIADTTDAVRDPQTLEILDITPCSFGGVSVITPGT NTSNQVAVLYQDVNCTEVPVAIHADQLTPTWRVYSTGSNVFQTRAGCLIGAEHVNNSY ECDIPIGAGICASYQTQTNSPRRARSVASQSIIAYTMSLGAENSVAYSNNSIAIPTNFTISVT TEILPVSMTKTSVDCTMYICGDSTECSNLLLQYGSFCTQLNRALTGIAVEQDKNTQEVFA QVKQIYKTPPIKDFGGFNFSQILPDPSKPSKRSFIEDLLFNKVTLADAGFIKQYGDCLGDIA ARDLICAQKFNGLTVLPPLLTDEMIAQYTSALLAGTITSGWTFGAGAALQIPFAMQMAY RFNGIGVTQNVLYENQKLIANQFNSAIGKIQDSLSSTASALGKLQDVVNQNAQALNTLV KQLSSNFGAISSVLNDILSRLDKVEAEVQIDRLITGRLQSLQTYVTQQLIRAAEIRASANLA ATKMSECVLGQSKRVDFCGKGYHLMSFPQSAPHGVVFLHVTYVPAQEKNFTTAPAICH DGKAHFPREGVFVSNGTHWFVTQRNFYEPQIITTDNTFVSGNCDVVIGIVNNTVYDPLQP ELDSFKEELDKYFKNHTSPDVDLGDISGINASVVNIQKEIDRLNEVAKNLNESLIDLQELG KYEQYIKWPWYIWLGFIAGLIAIVMVTIMLCCMTSCCSCLKGCCSCGSCCKFDEDDSEPV LKGVKLHYT (SEQ ID NO:51). In some embodiments, the SARS-CoV-2 spike protein (S) comprises an amino acid sequence of SEQ ID NO:51.

In some embodiments, the SARS-CoV-2 spike protein (S) or an immunogenic fragment thereof comprises a trimerized SARS-CoV-2 receptor-binding domain. In some embodiments, the SARS-CoV-2 spike protein (S) or an immunogenic fragment thereof comprises prefusion stabilized membrane-anchored SARS-CoV-2 full-length spike protein. In some embodiments, the SARS-CoV-2 spike protein (S) or an immunogenic fragment thereof comprises a prefusion stabilized SARS-CoV-2 spike protein.

In some embodiments, the SARS-CoV-2 spike protein (S) or an immunogenic fragment thereof comprises the receptor binding domain of the SARS-CoV-2 spike protein. In some embodiments, the receptor binding domain comprises the amino acid sequence of TNLCPFGEVFNATRFASVYAWNRKRISNCVADYSVLYNSASFSTFKCYGVSPTKLNDLC FTNVYADSFVIRGDEVRQIAPGQTGKIADYNYKLPDDFTGCVIAWNSNNLDSKVGGNYN YLYRLFRKSNLKPFERDISTEIYQAGSTPCNGVEGFNCYFPLQSYGFQPTNGVGYQPYRV VVLSFELLHAPATVCGP (SEQ ID NO:32). In some embodiments, the receptor binding domain comprises the amino acid sequence of NITNLCPFGEVFNATRFASVYAWNRKRISNCVADYSVLYNSASFSTFKCYGVSPTKLNDL CFTNVYADSFVIRGDEVRQIAPGQTGKIADYNYKLPDDFTGCVIAWNSNNLDSKVGGNY NYLYRLFRKSNLKPFERDISTEIYQAGSTPCNGVEGFNCYFPLQSYGFQPTNGVGYQPYR VVVLSFELLHAPATVCGP (SEQ ID NO:33). In some embodiments, the receptor binding domain comprises the amino acid sequence of FPNITNLCPFGEVFNATRFASVYAWNRKRISNCVADYSVLYNSASFSTFKCYGVSPTKLN DLCFTNVYADSFVIRGDEVRQIAPGQTGKIADYNYKLPDDFTGCVIAWNSNNLDSKVGG NYNYLYRLFRKSNLKPFERDISTEIYQAGSTPCNGVEGFNCYFPLQSYGFQPTNGVGYQP YRVVVLSFELLHAPATVCGP (SEQ ID NO:53). In some embodiments, the receptor binding domain comprises the amino acid sequence of TNLCPFGEVFNATRFASVYAWNRKRISNCVADYSVLYNSASFSTFKCYGVSPTKLNDLC FTNVYADSFVIRGDEVRQIAPGQTGKIADYNYKLPDDFTGCVIAWNSNNLDSKVGGNYN YLYRLFRKSNLKPFERDISTEIYQAGSTPCNGVEGFNCYFPLQSYGFQPTNGVGYQPYRV VVLSFELLHAPATVCGPKKST (SEQ ID NO:54). In some embodiments, the receptor binding domain comprises the amino acid sequence of NITNLCPFGEVFNATRFASVYAWNRKRISNCVADYSVLYNSASFSTFKCYGVSPTKLNDL CFTNVYADSFVIRGDEVRQIAPGQTGKIADYNYKLPDDFTGCVIAWNSNNLDSKVGGNY NYLYRLFRKSNLKPFERDISTEIYQAGSTPCNGVEGFNCYFPLQSYGFQPTNGVGYQPYR VVVLSFELLHAPATVCGPKKST (SEQ ID NO:55). In some embodiments, the receptor binding domain comprises the amino acid sequence of FPNITNLCPFGEVFNATRFASVYAWNRKRISNCVADYSVLYNSASFSTFKCYGVSPTKLN DLCFTNVYADSFVIRGDEVRQIAPGQTGKIADYNYKLPDDFTGCVIAWNSNNLDSKVGG NYNYLYRLFRKSNLKPFERDISTEIYQAGSTPCNGVEGFNCYFPLQSYGFQPTNGVGYQP YRVVVLSFELLHAPATVCGPKKST (SEQ ID NO:56).

In some embodiments, the receptor binding domain comprises an amino acid sequence having at least 70%, at least 80%, at least 85%, at least 90%, at least 95%, at least 97%, at least 98% at least 99% or at least 100% identity with (SEQ ID NO:32). In some embodiments, the receptor binding domain comprises an amino acid sequence having at least 70%, at least 80%, at least 85%, at least 90%, at least 95%, at least 97%, at least 98% at least 99% or at least 100% identity with SEQ ID NO:33. In some embodiments, the receptor binding domain comprises an amino acid sequence having at least 70%, at least 80%, at least 85%, at least 90%, at least 95%, at least 97%, at least 98% at least 99% or at least 100% identity with SEQ ID NO:53. In some embodiments, the receptor binding domain comprises an amino acid sequence having at least 70%, at least 80%, at least 85%, at least 90%, at least 95%, at least 97%, at least 98% at least 99% or at least 100% identity with SEQ ID NO:54. In some embodiments, the receptor binding domain comprises an amino acid sequence having at least 70%, at least 80%, at least 85%, at least 90%, at least 95%, at least 97%, at least 98% at least 99% or at least 100% identity with SEQ ID NO:55. In some embodiments, the receptor binding domain comprises an amino acid sequence having at least 70%, at least 80%, at least 85%, at least 90%, at least 95%, at least 97%, at least 98% at least 99% or at least 100% identity with SEQ ID NO:56.

In some embodiments, the receptor binding domain comprises one or more engineered glycosylation site, wherein the engineered glycosylation site comprises the amino acid sequence of NXS or NXT, wherein X is not proline. In some embodiments, the one or more engineered glycosylation site is at an amino acid position corresponding to position 346, 357, 360, 370, 381, 386, 394, 428, 444, 458, 468, 481, 518, and/or 522 of SEQ ID NO:51. In some embodiments, the one or more engineered glycosylation site is at an amino acid position corresponding to position 357, 360, 381, 386, 394, 428, 518, and/or 522 of SEQ ID NO:51.

In some embodiments, the receptor binding domain comprises 1, 2, 3, 4, 5, 6, 7, or 8 engineered glycosylation sites.

In some embodiments, the receptor binding domain comprises an engineered glycosylation site at the amino acid positions corresponding to
a) positions 357, 381, 386, 394, and 528 of SEQ ID NO:51;
b) positions 357, 394, 428, 518, and 522 of SEQ ID NO:51;
c) positions 357, 394, 428, and 518 of SEQ ID NO:51;
d) positions 357, and 518 of SEQ ID NO:51;
e) positions 357, 386, and 518 of SEQ ID NO:51;
f) positions 357, 386, 394, 428, and 518 of SEQ ID NO:51;
g) positions 386, 394, 518, and 522 of SEQ ID NO:51;
h) positions 357, 381, 386, 394, 428, 518, and 522 of SEQ ID NO:51;
i) positions 357, 386, 394, 428, 518, and 522 of SEQ ID NO:51;
j) positions 357, 381, 394, and 428 of SEQ ID NO:51; or
k) positions 357, 381, 394, and 518 of SEQ ID NO:51.

In some embodiments, the receptor binding domain comprises an engineered glycosylation site at the amino acid positions corresponding to
a) positions 357, 386, 394, 428, and 518 of SEQ ID NO:51;
b) positions 357, 386, 394, and 518 of SEQ ID NO:51;
c) positions 357, 386, and 518 of SEQ ID NO:51.

In some embodiments, the receptor binding domain comprises an engineered glycosylation site at the amino acid positions corresponding to positions 357, 386, 394, 428, and 518 of SEQ ID NO:51.

In some embodiments, the receptor binding domain comprises an engineered glycosylation site at the amino acid positions corresponding to positions 357, and 518 of SEQ ID NO:51.

In some embodiments, the receptor binding domain comprises an engineered glycosylation site at the amino acid positions corresponding to
a) positions 357, and 518 of SEQ ID NO:51;
b) positions 346, 357, 428, and 518 of SEQ ID NO:51;
c) positions 357, 386, 394, and 518 of SEQ ID NO:51;
d) positions 346, 357, 386, 428, and 518 of SEQ ID NO:51;
e) positions 357, 428, and 518 of SEQ ID NO:51;
f) positions 357, 386, 428, and 518 of SEQ ID NO:51; or
g) positions 357, 394, and 518 of SEQ ID NO:51.

### Membrane Tether

In some embodiments, a fusion polypeptide described herein further comprises an amino acid sequence that targets the fusion polypeptide to the cell surface. In some embodiments, the amino acid sequence that targets the fusion polypeptide to the cell surface comprises a GPI anchor signal sequence. In some embodiments, the amino acid sequence that targets the fusion polypeptide to the cell surface comprises a transmembrane domain.

In some embodiments, the fusion polypeptide comprises an amino acid sequence that targets the fusion polypeptide to the cell surface. In some embodiments, the amino acid sequence that targets the fusion polypeptide to the cell surface comprises a GPI anchor signal sequence.

In some embodiments, the fusion polypeptide comprises an amino acid sequence that targets the fusion polypeptide to the cell surface. In some embodiments, the amino acid sequence that targets the fusion polypeptide to the cell surface comprises a transmembrane domain.

In some embodiments, the transmembrane domain (TM) is from HIV Env of the BG505 isolate (KIFIMIVGGLIGLRIVFAVLSVIHRVR; SEQ ID NO:6), but any TM domain will suffice. For example, other TM domains could include the TM from SARS-CoV-2 (KWPWYIWLGFIAGLIAIVMVTIML; SEQ ID NO: 7) or the TM from VSV-G (KSSIASFFFIIGLIIGLFLVLR; SEQ ID NO: 8).

In some embodiments, the fusion polypeptide comprises an amino acid sequence that targets the fusion polypeptide to the cell surface. transmembrane domain comprises an HIV Env transmembrane domain, a SARS-CoV-2 transmembrane domain, or a VSV-G transmembrane domain. In some embodiments, the HIV Env transmembrane domain comprises the amino acid sequence of SEQ ID NO:6, the SARS-CoV-2 transmembrane domain comprises the amino acid sequence of SEQ ID NO:7, and the VSV-G transmembrane domain comprises the amino acid sequence of SEQ ID NO:8.

In some embodiments, the transmembrane domain comprises a VSV-G transmembrane domain. In some embodiments, the VSV-G transmembrane domain comprises the amino acid sequence of SEQ ID NO:8.

In some embodiments, the viral polypeptide and the transmembrane domain are directly linked. In some embodiments, the viral polypeptide and the transmembrane domain are separated by a linker peptide. In some embodiments, the linker comprises no more than 10 or no more than 5 amino acid residues. In some embodiments, the linker comprises one or more repeats of the GGS (SEQ ID NO:43) or GGGS (SEQ ID NO:44) sequence. In some embodiments, the linker comprises the amino acid sequence of GGS (SEQ ID NO:43), GGSGGS (SEQ ID NO:45), GGSGGSGGS (SEQ ID NO:46), GGGS (SEQ ID NO: 44), GGGSGGGS (SEQ ID NO:47), or GGGSGGGSGGGS (SEQ ID NO:34).

In some embodiments, in a fusion polypeptide described herein the viral polypeptide is closer to the N terminus than the transmembrane domain.

In some embodiments, multimerization domains could be added in order to display clusters of the fusion polypeptide on the membrane surface. While tethering to the membrane already should provide a multivalent array of RBDs for B cell interaction, fusion to multimerization domains can enhance the local fusion polypeptide density and concomitantly enhance B cell activation. Such multimerization domains could be added either to a linker, or to the C-terminus of the construct after the TM domain. In some embodiments, the multimerization domain is added after the TM domain. Without being bound by any specific theory, this arrangement would hide the domain from B cell recognition and thus avoid generating non-RBD antibody responses. Examples of small multimerization domains (with fewer than 50 amino acids) include trimerization motifs like the coiled-coil GCN4 (PDB ID: 1GCN) or the trimeric fibritin foldon, or tetramerization motifs like the tetrameric variant of GCN4 in PDB ID 1GCL, or the heptameric coil in PDB ID: 4PNA or the octameric coil in PDB ID: 6G67. Larger multimerization domains with >100 amino acids which include a larger number of CD4 T helper epitopes could also be included. In some embodiments, a lumazine synthase domain that self-assembles into a pentamer can be fused C-terminal to the TM domain, to serve a dual purpose of providing additional T help and providing multimerization. Another example is the protein PH0250 that assembles into a 12-mer ring in PDB ID: 2EKD.

### Self-Assembling Domain Capable of Forming a Nanoparticle

In some embodiments, a fusion polypeptide described herein further comprises a self-assembling domain capable of forming a nanoparticle. In some embodiments, the self-assembling domain comprises a type II 3-Dehydroquinase, ferritin or lumazine synthase. In some embodiments, the self-assembling domain comprises a type II 3-Dehydroquinase polypeptide comprising one or more engineered glycosylation site. In some embodiments, the self-assembling domain comprises a Thermus thermophilus type type II 3-Dehydroquinase, optionally comprising one or more engineered glycosylation site.

In some embodiments, the self-assembling domain comprises a type II 3-Dehydroquinase, ferritin or lumazine synthase. In some embodiments, the self-assembling domain comprises a Thermus thermophilus, Mycobacterium tuberculosis, Streptomyces coelicolor, Acinetobacter baumannii, Yersinia pestis, Bacillus subtilis, Proprionibacterium acnes, Acidithiobacillus caldus, Zymomonas mobilus, Helicobacter pylori, Pseudomonas aeruginosa, Candida albicans, or Psychromonas ingrahamii type II 3-Dehydroquinase polypeptide.

In some embodiments, the self-assembling domain comprises a Thermus thermophilus type II 3-Dehydroquinase polypeptide. In some embodiments, the Thermus thermophilus type II 3-Dehydroquinase polypeptide comprises an amino acid sequence having at least 70%, at least 80%, at least 85%, at least 90%, at least 95%, at least 97%, at least 98% at least 99% or at least 100% identity with MVLILNGPNLNLLGRREPEVYGRTTLEELEALCEAWGAELGLGVVFRQTNYEGQLIEWV QNAWQEGFLAIVLNPGALTHYSYALLDAIRAQPLPVVEVHLTNLHAREEFRRHSVTAPA ARGIVSGFGPLSYKLALVYLAETLEVGGEGF (SEQ ID NO:48). In some embodiments, the Thermus thermophilus type II 3-Dehydroquinase polypeptide comprises the amino acid sequence of SEQ ID NO:48.

In some embodiments, the 3-Dehydroquinase polypeptide comprises one or more engineered glycosylation site, wherein the engineered glycosylation site comprises the amino acid sequence of NXS or NXT, wherein X is not proline. In some embodiments, the one or more engineered glycosylation site is at an amino acid position corresponding to position 1, 25, 32, 49, and/or 63 of NGSVLILNGPNLNLLGRREPEVYGNTTLEELNASAEAWGAELGLGVVFNQTNYEGQLIE WVQNASQEGFLAIVLNPGALTHYSYALLDAIRAQPLPVVEVHLTNLHAREEFRRHSVTA PAARGIVSGFGPLSYKLALVYLAETLEVGGEGF (SEQ ID NO:52). In some embodiments, the 3-Dehydroquinase polypeptide comprises 1, 2, 3, 4, or 5 engineered glycosylation sites.

In some embodiments, the 3-Dehydroquinase polypeptide comprises the amino acid sequence of SEQ ID NO:52.

In some embodiments, a fusion polypeptide described herein comprises from the N terminus to the C terminus VP-SAD, SAD-VP, VP-SAD-VP, wherein VP and SAD corresponds to the at least one viral polypeptide, and self-assembling domain, respectively.

In some embodiments, the viral polypeptide comprises the receptor binding domain of the SARS-CoV-2 spike protein comprising one or more engineered glycosylation site.

In some embodiments, the VP and SAD are directly linked, wherein VP and SAD corresponds to the at least one viral polypeptide, and self-assembling domain, respectively. In some embodiments, the fusion polypeptide comprises one or more linkers linking the VP and SAD. In some embodiments, the fusion polypeptide comprises one or more linkers linking the VP and SAD. In some embodiments, the one or more linker independently comprises no more than 10 or no more than 5 amino acid residues. In some embodiments, the one or more linker independently comprises one or more repeats of the GGS (SEQ ID NO:43) or GGGS (SEQ ID NO:44) sequence. In some embodiments, the one or more linker independently comprises the amino acid sequence of GGS (SEQ ID NO:43), GGSGGS (SEQ ID NO:45), GGSGGSGGS (SEQ ID NO:46), GGGS (SEQ ID NO: 44), GGGSGGGS (SEQ ID NO:47), or GGGSGGGSGGGS (SEQ ID NO:34).

### Immunogenic Polypeptide Comprising One or More MHC Class II T Cell Epitope

In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40), ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42).

In some embodiments, the MHC class II T cell epitope comprises an amino acid sequence selected from the group consisting of
(a) ATPHFDYIASEVSKG (SEQ ID NO:37) comprising 0, 1, 2, 3, 4, or 5 substitutions,
(b) FGVITADTLEQAIER (SEQ ID NO:38) comprising 0, 1, 2, 3, 4, or 5 substitutions,
(c) FDYIASEVSKGLADL (SEQ ID NO:39) comprising 0, 1, 2, 3, 4, or 5 substitutions,
(d) ATPHFDYIASEVSKGLADL (SEQ ID NO:40) comprising 0, 1, 2, 3, 4, or 5 substitutions,
(e) 9, 10, 11, 12, 13, 14, or 15 consecutive residues of ATPHFDYIASEVSKG (SEQ ID NO:37),
(f) 9, 10, 11, 12, 13, 14, or 15 consecutive residues of FGVITADTLEQAIER (SEQ ID NO:38),
(g) 9, 10, 11, 12, 13, 14, or 15 consecutive residues of FDYIASEVSKGLADL (SEQ ID NO:39), and
(h) 10, 11, 12, 13, 14, 15, 16, 17, 18, or 19 consecutive residues of ATPHFDYIASEVSKGLADL (SEQ ID NO:40).

In some embodiments, the MHC class II T cell epitope comprises an amino acid sequence selected from the group consisting of
(a) 9, 10, 11, 12, 13, 14, or 15 consecutive residues of ATPHFDYIASEVSKG (SEQ ID NO:37),
(b) 9, 10, 11, 12, 13, 14, or 15 consecutive residues of FGVITADTLEQAIER (SEQ ID NO:38),
(c) 9, 10, 11, 12, 13, 14, or 15 consecutive residues of FDYIASEVSKGLADL (SEQ ID NO:39), and
(d) 10, 11, 12, 13, 14, 15, 16, 17, 18, or 19 consecutive residues of ATPHFDYIASEVSKGLADL (SEQ ID NO:40).

In some embodiments, the MHC class II T cell epitope comprises an amino acid sequence selected from the group consisting of
(a) ATPHFDYIASEVSKG (SEQ ID NO:37),
(b) FGVITADTLEQAIER (SEQ ID NO:38),
(c) FDYIASEVSKGLADL (SEQ ID NO:39), and
(d) ATPHFDYIASEVSKGLADL (SEQ ID NO:40).

In some embodiments, the immunogenic polypeptide comprises at least 2 MHC class II T cell epitopes. In some embodiments, the at least 2 MHC class II T cell epitopes comprise the amino acid sequences of
(a) ATPHFDYIASEVSKG (SEQ ID NO:37) comprising 0, 1, 2, 3, 4, or 5 substitutions; and
(b) FGVITADTLEQAIER (SEQ ID NO:38) comprising 0, 1, 2, 3, 4, or 5 substitutions.

In some embodiments, the at least 2 MHC class II T cell epitopes comprise the amino acid sequences of
(a) ATPHFDYIASEVSKG (SEQ ID NO:37); and
(b) FGVITADTLEQAIER (SEQ ID NO:38).

In some embodiments, the at least 2 MHC class II T cell epitopes comprise the amino acid sequences of
(a) ATPHFDYIASEVSKGLADL (SEQ ID NO:40) comprising 0, 1, 2, 3, 4, or 5 substitutions; and
(b) FGVITADTLEQAIER (SEQ ID NO:38) comprising 0, 1, 2, 3, 4, or 5 substitutions.

In some embodiments, the at least 2 MHC class II T cell epitopes comprise the amino acid sequences of (a) ATPHFDYIASEVSKGLADL (SEQ ID NO:40); and (b) FGVITADTLEQAIER (SEQ ID NO:38).

In some embodiments, the at least 2 MHC class II T cell epitopes are directly linked in any order. In some embodiments, the at least 2 MHC class II T cell epitopes are in any order and are separated by a linker peptide. In some embodiments, the linker comprises no more than 10 or no more than 5 amino acid residues. In some embodiments, the linker comprises one or more repeats of the GGS (SEQ ID NO:43) or GGGS (SEQ ID NO:44) sequence. In some embodiments, the linker comprises the amino acid sequence of GGS (SEQ ID NO:43), GGSGGS (SEQ ID NO:45), GGSGGSGGS (SEQ ID NO:46), GGGS (SEQ ID NO: 44), GGGSGGGS (SEQ ID NO:47), or GGGSGGGSGGGS (SEQ ID NO:34).

In some embodiments, the immunogenic polypeptide comprises an amino acid sequence selected from the group consisting of
(a) ATPHFDYIASEVSKGLADL (SEQ ID NO:40) comprising 0, 1, 2, 3, 4, or 5 substitutions;
(b) ATPHFDYIASEVSKGLADL (SEQ ID NO:40);
(c) ATPHFDYIASEVSKGLADLGGSFGVITADTLEQAIER (SEQ ID NO:41) comprising 0, 1, 2, 3, 4, or 5 substitutions;
(d) amino acid sequence having at least 70%, at least 80%, at least 85%, at least 90%, at least 95%, at least 97%, at least 98% at least 99% or at least 100% identity with ATPHFDYIASEVSKGLADLGGSFGVITADTLEQAIER (SEQ ID NO:41);
(e) ATPHFDYIASEVSKGLADLGGSFGVITADTLEQAIER (SEQ ID NO: 41);
(f) ATPHFDYIASEVSKGLADLSLELRKPITFGVITADTLEQAIER (SEQ ID NO:42) comprising 0, 1, 2, 3, 4, or 5 substitutions;
(g) amino acid sequence having at least 70%, at least 80%, at least 85%, at least 90%, at least 95%, at least 97%, at least 98% at least 99% or at least 100% identity with ATPHFDYIASEVSKGLADLSLELRKPITFGVITADTLEQAIER (SEQ ID NO:42); and
(h) ATPHFDYIASEVSKGLADLSLELRKPITFGVITADTLEQAIER (SEQ ID NO:42).

In some embodiments, the immunogenic polypeptide comprises an amino acid sequence selected from the group consisting of
(a) ATPHFDYIASEVSKGLADL (SEQ ID NO:40);
(b) ATPHFDYIASEVSKGLADLGGSFGVITADTLEQAIER (SEQ ID NO:41); and
(c) ATPHFDYIASEVSKGLADLSLELRKPITFGVITADTLEQAIER (SEQ ID NO:42).

In some embodiments, the immunogenic polypeptide comprises the amino acid sequence of ATPHFDYIASEVSKGLADL (SEQ ID NO:40).

In some embodiments, the immunogenic polypeptide comprises the amino acid sequence of ATPHFDYIASEVSKGLADLGGSFGVITADTLEQAIER (SEQ ID NO:41).

In some embodiments, the MHC class II T cell epitope comprises the amino acid sequence of AKFVAAWTLKAAA (SEQ ID NO:36).

Additional broadly reactive CD4 T helper epitopes can be added. Examples of such broadly reactive CD4 T helper epitopes include, without limitation:
(a) the TT p2 epitope 830-QYIKANSKFIGITE-843 (Falugi et al. 2001; SEQ ID NO:23) or the related TT epitope 826-NILMQYIKANSK-837 (Wantuch et al. 2020; SEQ ID NO:24) or those two combined into 826-NILMQYIKANSKFIGITE-843 (SEQ ID NO:25), each of which is incorporated herein by reference in its entirety;
(b) the TT p32 epitope 1174-LKFIIKRYTPNNEIDS-1189 (Falugi et al. 2001; SEQ ID NO:26) or the related TT epitope 1169-LYNGLKFIIKR-1179 (Wantuch et al. 2020; SEQ ID NO:27) or those two combined into 1169-LYNGLKFIIKRYTPNNEIDS-1189 (SEQ ID NO:28), each of which is incorporated herein by reference in its entirety;
(c) the CRM197 epitope 299-KTTAALSILPGIGS-312 (Wantuch et al. 2020; SEQ ID NO:29), which is incorporated herein by reference in its entirety;
(d) the HBsAg_19-33 epitope FFLLTRILTIPQSLD (Celis JImmunol 1988; Greenstein et al J Virol 1992; SEQ ID NO:30), which is incorporated herein by reference in its entirety;
(e) the T* epitope from P. falciparum strain NF54 residues 326-345 EYLNKIQNSLSTEWSCSVT (Moreno et al. J Immunol 1993; SEQ ID NO:31), which is incorporated herein by reference in its entirety.

### Fusion Polypeptide Architecture

In some embodiments, a fusion polypeptide described herein comprises from the N terminus to the C terminus VP-IP-TM or VP-TM-IP, wherein VP, IP and TM corresponds to the at least one viral polypeptide, at least one immunogenic polypeptide, and transmembrane domain, respectively. In some embodiments, a fusion polypeptide described herein comprises from the N terminus to the C terminus IP1-VP-IP2-TM, wherein VP, IP and TM corresponds to the at least one viral polypeptide, at least one immunogenic polypeptide, and transmembrane domain, respectively. In some embodiments, a fusion polypeptide described herein comprises from the N terminus to the C terminus IP1-VP-TM-IP2, wherein VP, IP and TM corresponds to the at least one viral polypeptide, at least one immunogenic polypeptide, and transmembrane domain, respectively. In some embodiments IP1 and IP2 comprise the same sequence. In some embodiments IP1 and IP2 comprise different sequences. In some embodiments, IP1 comprises the amino acid sequence of ATPHFDYIASEVSKGLADL (SEQ ID NO:40) and IP2 comprises the amino acid sequence of FGVITADTLEQAIER (SEQ ID NO:38). In some embodiments, IP1 comprises the amino acid sequence of FGVITADTLEQAIER (SEQ ID NO:38) and IP2 comprises the amino acid sequence of ATPHFDYIASEVSKGLADL (SEQ ID NO:40). In some embodiments, the viral polypeptide comprises a receptor binding domain of the SARS-CoV-2 spike protein comprising one or more engineered glycosylation site described herein.

In some embodiments, the VP, IP and TM are directly linked. In some embodiments, the fusion polypeptide comprises one or more linkers linking the VP, IP and/or TM. In some embodiments, the fusion polypeptide comprises linkers linking the VP, IP and TM. T In some embodiments, the one or more linker or linkers independently comprise no more than 10 or no more than 5 amino acid residues. In some embodiments, the one or more linker or linkers independently comprise one or more repeats of the GGS (SEQ ID NO:43) or GGGS (SEQ ID NO:44) sequence. In some embodiments, the one or more linker or linkers independently comprise the amino acid sequence of GGS (SEQ ID NO:43), GGSGGS (SEQ ID NO:45), GGSGGSGGS (SEQ ID NO:46), GGGS (SEQ ID NO: 44), GGGSGGGS (SEQ ID NO:47), or GGGSGGGSGGGS (SEQ ID NO:34).

In some embodiments, a fusion polypeptide described herein comprises from the N terminus to the C terminus VP-IP-SAD, SAD-IP-VP, VP-IP-SAD-IP-VP, wherein VP, IP and SAD corresponds to the at least one viral polypeptide, at least one immunogenic polypeptide, and self-assembling domain, respectively. In some embodiments, the fusion polypeptide comprises two or more immunogenic polypeptides, and the two or more immunogenic polypeptides comprise the same amino acid sequence. In some embodiments, the fusion polypeptide comprises two or more immunogenic polypeptides, and the two or more immunogenic polypeptides comprise different amino acid sequences. In some embodiments, the viral polypeptide comprises a receptor binding domain of the SARS-CoV-2 spike protein comprising one or more engineered glycosylation site described herein.

In some embodiments, a fusion polypeptide described herein comprises from the N terminus to the C terminus IP1-VP-IP2-SAD or SAD-IP1-VP-IP2, wherein VP, IP and SAD corresponds to the at least one viral polypeptide, at least one immunogenic polypeptide, and self-assembling domain, respectively. In some embodiments IP1 and IP2 comprise the same sequence. In some embodiments IP1 and IP2 comprise different sequences. In some embodiments, IP1 comprises the amino acid sequence of ATPHFDYIASEVSKGLADL (SEQ ID NO:40) and IP2 comprises the amino acid sequence of FGVITADTLEQAIER (SEQ ID NO:38). In some embodiments, IP1 comprises the amino acid sequence of FGVITADTLEQAIER (SEQ ID NO:38) and IP2 comprises the amino acid sequence of ATPHFDYIASEVSKGLADL (SEQ ID NO:40). In some embodiments, the viral polypeptide comprises a receptor binding domain of the SARS-CoV-2 spike protein comprising one or more engineered glycosylation site described herein.

In some embodiments, a fusion polypeptide described herein comprises from the N terminus to the C terminus VP-IP1-SAD-IP2 or IP1-SAD-IP2-VP, wherein VP, IP and SAD corresponds to the at least one viral polypeptide, at least one immunogenic polypeptide, and self-assembling domain, respectively. In some embodiments, a fusion polypeptide described herein comprises from the N terminus to the C terminus VP-IP1-SAD-IP2-VP, wherein VP, IP and SAD corresponds to the at least one viral polypeptide, at least one immunogenic polypeptide, and self-assembling domain, respectively. In some embodiments, a fusion polypeptide described herein comprises from the N terminus to the C terminus VP-IP1-SAD-IP2-VP, wherein VP, IP and SAD corresponds to the at least one viral polypeptide, at least one immunogenic polypeptide, and self-assembling domain, respectively. In some embodiments IP1 and IP2 comprise the same sequence. In some embodiments IP1 and IP2 comprise different sequences. In some embodiments, IP1 comprises the amino acid sequence of ATPHFDYIASEVSKGLADL (SEQ ID NO:40) and IP2 comprises the amino acid sequence of FGVITADTLEQAIER (SEQ ID NO:38). In some embodiments, IP1 comprises the amino acid sequence of FGVITADTLEQAIER (SEQ ID NO:38) and IP2 comprises the amino acid sequence of ATPHFDYIASEVSKGLADL (SEQ ID NO:40). In some embodiments, the viral polypeptide comprises a receptor binding domain of the SARS-CoV-2 spike protein comprising one or more engineered glycosylation site described herein.

In some embodiments, a fusion polypeptide described herein comprises from the N terminus to the C terminus IP1-VP-IP2-SAD-IP3-VP-IP4, IP1-VP-IP2-SAD-IP3, IP1-SAD-IP2-VP-IP3, wherein VP, IP and SAD corresponds to the at least one viral polypeptide, at least one immunogenic polypeptide, and self-assembling domain, respectively. In some embodiments, the fusion polypeptide comprises two or more immunogenic polypeptides, and the two or more immunogenic polypeptides comprise the same amino acid sequence. In some embodiments, the fusion polypeptide comprises two or more immunogenic polypeptides, and the two or more immunogenic polypeptides comprise different amino acid sequences. In some embodiments IP1 and IP2 comprise the same sequence. In some embodiments, IP1 and IP2 comprise different sequences. In some embodiments, IP1 comprises the amino acid sequence of ATPHFDYIASEVSKGLADL (SEQ ID NO:40) and IP2 comprises the amino acid sequence of FGVITADTLEQAIER (SEQ ID NO:38). In some embodiments, IP1 comprises the amino acid sequence of FGVITADTLEQAIER (SEQ ID NO:38) and IP2 comprises the amino acid sequence of ATPHFDYIASEVSKGLADL (SEQ ID NO:40). In some embodiments, the fusion polypeptide comprises two or more viral polypeptides, and the two or more viral polypeptides comprise the same amino acid sequence. In some embodiments, the fusion polypeptide comprises two or more viral polypeptides, and the two or more viral polypeptides comprise different amino acid sequences. In some embodiments, the viral polypeptide comprises a receptor binding domain of the SARS-CoV-2 spike protein comprising one or more engineered glycosylation site described herein.

In some embodiments, the VP, IP and SAD are directly linked. In some embodiments, the fusion polypeptide comprises one or more linker linking the VP, IP and/or SAD. In some embodiments, the fusion polypeptide comprises linkers linking the VP, IP and SAD. In some embodiments, the one or more linker independently comprises no more than 10 or no more than 5 amino acid residues. In some embodiments, the one or more linker independently comprises one or more repeats of the GGS (SEQ ID NO:43) or GGGS (SEQ ID NO:44) sequence. In some embodiments, the one or more linker independently comprises the amino acid sequence of GGS (SEQ ID NO:43), GGSGGS (SEQ ID NO:45), GGSGGSGGS (SEQ ID NO:46), GGGS (SEQ ID NO: 44), GGGSGGGS (SEQ ID NO:47), or GGGSGGGSGGGS (SEQ ID NO:34).

### Signal Peptide

In some embodiments, a fusion polypeptide described herein further comprises a signal peptide. In some embodiments, a secretion signal sequence from the pHLsec vector (MGILPSPGMPALLSLVSLLSVLLMGCVAETG; SEQ ID NO: 5) is used, but any secretion signal sequence can be used. The signal sequence will be cleaved during expression/secretion and is not present in the final expressed protein product. The embodiments contained herein are not limited to this particular signal sequence as different signal sequences could be used to serve the same purpose. In some embodiments, the signal peptide comprises the amino acid sequence of SEQ ID NO: 5.

In some embodiments, a fusion polypeptide described herein further comprises a His tag. In some embodiments, the His tag is at the C terminal end of the fusion polypeptide

In some embodiments, a fusion polypeptide described herein further comprises the amino acid sequence of HGKHGK (SEQ ID NO:35). In some embodiments, the HGKHGK (SEQ ID NO:35) sequence is at the C terminal end of the fusion polypeptide.

In some embodiments, a fusion polypeptide described herein comprises a cysteine residue at their N- or C-terminus that is capable of being conjugated to a phosphoserine group or chemically analogous group for the purpose of targeting the fusion polypeptide to a metal hydroxide adjuvant (e.g. alum) for improved immunogenicity. See, e.g., Moyer et al. Nature Medicine 26, pages 430-440 (2020), which is incorporated herein by reference in its entirety.

### Exemplary Fusion Polypeptides

In some embodiments, a fusion polypeptide described herein comprises an amino acid sequence that has at least 70%, at least 80%, at least 85%, at least 90%, at least 95%, at least 97%, at least 98% at least 99% or at least 100% identity with

(a)
(SEQ ID NO: 57)
TNLCPFGEVFNATRFASVYAWNRKNISNCVADYSVLYNSASFSTFKCY
GVSPTNLTDLCFTNVSADSFVIRGDEVRQIAPGQTGKIADYNYKLPDN
FTGCVIAWNSNNLDSKVGGNYNYLYRLFRKSNLKPFERDISTEIYQAG
STPCNGVEGFNCYFPLQSYGFQPTNGVGYQPYRVVVLSFELNHTPATV
CGPGGSAKFVAAWTLKAAAGGSKSSIASFFFIIGLIIGLFLVLR,
(b)
(SEQ ID NO: 59)
TNLCPFGEVFNATRFASVYAWNRKNISNCVADYSVLYNSASFSTFKCY
GVSPTNLTDLCFTNVSADSFVIRGDEVRQIAPGQTGKIADYNYKLPDN
FTGCVIAWNSNNLDSKVGGNYNYLYRLFRKSNLKPFERDISTEIYQAG
STPCNGVEGFNCYFPLQSYGFQPTNGVGYQPYRVVVLSFELNHTPATV
CGPGGSGGSGGSGGSGGSGGSKSSIASFFFIIGLIIGLFLVLR,
(c)
(SEQ ID NO: 61)
TNLCPFGEVENATRFASVYAWNRKNISNCVADYSVLYNSASFSTFKCY
GVSPTNLTDLCFTNVSADSFVIRGDEVRQIAPGQTGKIADYNYKLPDN
FTGCVIAWNSNNLDSKVGGNYNYLYRLFRKSNLKPFERDISTEIYQAG
STPCNGVEGFNCYFPLQSYGFQPTNGVGYQPYRVVVLSFELNHTPATV
CGPGGSGGSGGSAKFVAAWTLKAAAGGSGGSGGSKSSIASFFFIIGLI
IGLFLVLR,
(d)
(SEQ ID NO: 63)
TNLCPFGEVFNATRFASVYAWNRKNISNCVADYSVLYNSASFSTFKCY
GVSPTNLTDLCFTNVSADSFVIRGDEVRQIAPGQTGKIADYNYKLPDN
FTGCVIAWNSNNLDSKVGGNYNYLYRLFRKSNLKPFERDISTEIYQAG
STPCNGVEGFNCYFPLQSYGFQPTNGVGYQPYRVVVLSFELNHTPATV
CGPGGSGGSGGSATPHFDYIASEVSKGLADLGGSGGSGGSKSSIASFF
FIIGLIIGLFLVLR,
(e)
(SEQ ID NO: 65)
TNLCPFGEVFNATRFASVYAWNRKNISNCVADYSVLYNSASFSTFKCY
GVSPTNLTDLCFTNVSADSFVIRGDEVRQIAPGQTGKIADYNYKLPDN
FTGCVIAWNSNNLDSKVGGNYNYLYRLFRKSNLKPFERDISTEIYQAG
STPCNGVEGFNCYFPLQSYGFQPTNGVGYQPYRVVVLSFELNHTPATV
CGPGGSGGSGGSATPHFDYIASEVSKGLADLGGSFGVITADTLEQAIE
RGGSGGSGGSKSSIASFFFIIGLIIGLFLVLR,
(f)
(SEQ ID NO: 67)
TNLCPFGEVFNATRFASVYAWNRKRISNCVADYSVLYNSASFSTFKCY
GVSPTKLNDLCFTNVYADSFVIRGDEVRQIAPGQTGKIADYNYKLPDD
FTGCVIAWNSNNLDSKVGGNYNYLYRLFRKSNLKPFERDISTEIYQAG
STPCNGVEGFNCYFPLQSYGFQPTNGVGYQPYRVVVLSFELLHAPATV
CGPGGSGGSGGSATPHFDYIASEVSKGLADLGGSFGVITADTLEQAIE
RGGSGGSGGSKSSIASFFFIIGLIIGLFLVLR,
(g)
(SEQ ID NO: 69)
TNLCPFGEVFNATRFASVYAWNRKNITNCVADYSVLYNSASFSTFKCY
GVSPTNLTDLCFTNVYADSFVIRGDEVRQIAPGQTGKIADYNYKLPDD
FTGCVIAWNSNNLDSKVGGNYNYLYRLFRKSNLKPFERDISTEIYQAG
STPCNGVEGFNCYFPLQSYGFQPTNGVGYQPYRVVVLSFELNHTPATV
CGPGGSGGSGGSATPHFDYIASEVSKGLADLGGSFGVITADTLEQAIE
RGGSGGSGGSKSSIASFFFIIGLIIGLFLVLR,
or
(h)
(SEQ ID NO: 71)
TNLCPFGEVFNATRFASVYAWNRKNITNCVADYSVLYNSASFSTFKCY
GVSPTNLTDLCFTNVSADSFVIRGDEVRQIAPGQTGKIADYNYKLPDD
FTGCVIAWNSNNLDSKVGGNYNYLYRLFRKSNLKPFERDISTEIYQAG
STPCNGVEGFNCYFPLQSYGFQPTNGVGYQPYRVVVLSFELNHTPATV
CGPGGSGGSGGSATPHFDYIASEVSKGLADLGGSFGVITADTLEQAIE
RGGSGGSGGSKSSIASFFFIIGLIIGLFLVLR.

In some embodiments, a fusion polypeptide described herein comprising the amino acid sequence of
(i) SEQ ID NO:57,
(j) SEQ ID NO:59,
(k) SEQ ID NO:61,
(l) SEQ ID NO:63,
(m) SEQ ID NO:65,
(n) SEQ ID NO:67,
(o) SEQ ID NO:69, or
(p) SEQ ID NO:71.

In some embodiments, a fusion polypeptide described herein comprises an amino acid sequence that has at least 70%, at least 80%, at least 85%, at least 90%, at least 95%, at least 97%, at least 98% at least 99% or at least 100% identity with

(a)
(SEQ ID NO: 73)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFGGGSATPHFDYIASEVSKGLADLGGSGGSGGSNITNLCPFG
EVFNATRFASVYAWNRKRISNCVADYSVLYNSASFSTFKCYGVSPTKL
NDLCFTNVYADSFVIRGDEVRQIAPGQTGKIADYNYKLPDDFTGCVIA
WNSNNLDSKVGGNYNYLYRLFRKSNLKPFERDISTEIYQAGSTPCNGV
EGFNCYFPLQSYGFQPTNGVGYQPYRVVVLSFELLHAPATVCGPGTKH
GKHGK,
(b)
(SEQ ID NO: 74)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFGGGSATPHFDYIASEVSKGLADLGGSFGVITADTLEQAIER
GGSGGSGGSNITNLCPFGEVFNATRFASVYAWNRKRISNCVADYSVLY
NSASFSTFKCYGVSPTKLNDLCFTNVYADSFVIRGDEVRQIAPGQTGK
IADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVL
SFELLHAPATVCGPGTKHHHHHH,
(c)
(SEQ ID NO: 75)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFGGGSATPHFDYIASEVSKGLADLGGSGGSGGSNITNLCPFG
EVFNATRFASVYAWNRKRISNCVADYSVLYNSASFSTFKCYGVSPTKL
NDLCFTNVYADSFVIRGDEVRQIAPGQTGKIADYNYKLPDDFTGCVIA
WNSNNLDSKVGGNYNYLYRLFRKSNLKPFERDISTEIYQAGSTPCNGV
EGFNCYFPLQSYGFQPTNGVGYQPYRVVVLSFELLHAPATVCGPGTKH
HHHHH,
(d)
(SEQ ID NO: 76)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFGTGTNLCPFGEVFNATRFASVYAWNRKRISNCVADYSVLYN
SASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFER
DISTEIYQAGSTPCNGVEGFNCYFPLQSYGFQPTNGVGYQPYRVVVLS
FELLHAPATVCGPGTKGSGSGS,
(e)
(SEQ ID NO: 77)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFGTGTNLCPFGEVFNATRFASVYAWNRKRISNCVADYSVLYN
SASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFER
DISTEIYQAGSTPCNGVEGFNCYFPLQSYGFQPTNGVGYQPYRVVVLS
FELLHAPATVCGPGTKKKKKKK,
(f)
(SEQ ID NO: 78)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFGTGTNLCPFGEVFNATRFASVYAWNRKRISNCVADYSVLYN
SASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFER
DISTEIYQAGSTPCNGVEGFNCYFPLQSYGFQPTNGVGYQPYRVVVLS
FELLHAPATVCGPGTKHGKHGK,
(g)
(SEQ ID NO: 79)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFGTGTNLCPFGEVFNATRFASVYAWNRKNITNCVADYSVLYN
SASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFER
DISTEIYQAGSTPCNGVEGFNCYFPLQSYGFQPTNGVGYQPYRVVVLS
FELNHTPATVCGPGTKHHHHHH,
(h)
(SEQ ID NO: 80)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFGTGTNLCPFGEVFNATNFSSVYAWNRKNITNCVADYSVLYN
SASFSTFKCYGVSPTKLNDLCFTNVYADSFVIRGDEVRQIAPGQTGKI
ADYNYKLPDNFTGCVIAWNSNNLDSKVGGNYNYLYRLFRKSNLKPFER
DISTEIYQAGSTPCNGVEGFNCYFPLQSYGFQPTNGVGYQPYRVVVLS
FELNHTPATVCGPGTKHHHHHH,
(i)
(SEQ ID NO: 81)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFGTGTNLCPFGEVFNATNFSSVYAWNRKNITNCVADYSVLYN
SASFSTFKCYGVSPTKLNDLCFTNVYADSFVIRGDEVRQIAPGQTGKI
ADYNYKLPDNFTGCVIAWNSNNLDSKVGGNYNYLYRLFRKSNLKPFER
DISTEIYQAGSTPCNGVEGFNCYFPLQSYGFQPTNGVGYQPYRVVVLS
FELNHTPATVCGPGTKHGKHGK,
(j)
(SEQ ID NO: 82-84)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFXNITNLCPFGEVFNATRFASVYAWNRKNITNCVADYSVLYN
SASFSTFKCYGVSPTNLTDLCFTNVSADSFVIRGDEVRQIAPGQTGKI
ADYNYKLPDDFTGCVIAWNSNNLDSKVGGNYNYLYRLFRKSNLKPFER
DISTEIYQAGSTPCNGVEGFNCYFPLQSYGFQPTNGVGYQPYRVVVLS
FELNHTPATVCGPGTKHGKHGK
wherein X is GTG, GGGSATPHFDYIASEVSKGLADLGGSGGSG
GS or GGGSATPHFDYIASEVSKGLADLGGSFGVITADTLEQAIERG
GSGGSGGS,
(k)
(SEQ ID NO: 85-87)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFXNITNLCPFGEVFNATNFSSVYAWNRKNITNCVADYSVLYN
SASFSTFKCYGVSPTKLNDLCFTNVYADSFVIRGDEVRQIAPGQTGKI
ADYNYKLPDNFTGCVIAWNSNNLDSKVGGNYNYLYRLFRKSNLKPFER
DISTEIYQAGSTPCNGVEGFNCYFPLQSYGFQPTNGVGYQPYRVVVLS
FELNHTPATVCGPGTKHGKHGK
wherein X is GTG, GGGSATPHFDYIASEVSKGLADLGGSGGSG
GS or GGGSATPHFDYIASEVSKGLADLGGSFGVITADTLEQAIERG
GSGGSGGS,
(l)
(SEQ ID NO: 88-90)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFXNITNLCPFGEVFNATNFSSVYAWNRKNITNCVADYSVLYN
SASFSTFKCYGVSPTNLTDLCFTNVYADSFVIRGDEVRQIAPGQTGKI
ADYNYKLPDNFTGCVIAWNSNNLDSKVGGNYNYLYRLFRKSNLKPFER
DISTEIYQAGSTPCNGVEGFNCYFPLQSYGFQPTNGVGYQPYRVVVLS
FELNHTPATVCGPGTKHGKHGK
wherein X is GTG, GGGSATPHFDYIASEVSKGLADLGGSGGSG
GS or GGGSATPHFDYIASEVSKGLADLGGSFGVITADTLEQAIERG
GSGGSGGS,
(m)
(SEQ ID NO: 91-93)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFXNITNLCPFGEVFNATRFASVYAWNRKNITNCVADYSVLYN
SASFSTFKCYGVSPTKLNDLCFTNVYADSFVIRGDEVRQIAPGQTGKI
ADYNYKLPDNFTGCVIAWNSNNLDSKVGGNYNYLYRLFRKSNLKPFER
DISTEIYQAGSTPCNGVEGFNCYFPLQSYGFQPTNGVGYQPYRVVVLS
FELNHTPATVCGPGTKHGKHGK
wherein X is GTG, GGGSATPHFDYIASEVSKGLADLGGSGGSG
GS or GGGSATPHFDYIASEVSKGLADLGGSFGVITADTLEQAIERG
GSGGSGGS,
(n)
(SEQ ID NO: 94-96)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFXNITNLCPFGEVFNATRFASVYAWNRKNITNCVADYSVLYN
SASFSTFKCYGVSPTNLTDLCFTNVYADSFVIRGDEVRQIAPGQTGKI
ADYNYKLPDNFTGCVIAWNSNNLDSKVGGNYNYLYRLFRKSNLKPFER
DISTEIYQAGSTPCNGVEGFNCYFPLQSYGFQPTNGVGYQPYRVVVLS
FELNHTPATVCGPGTKHGKHGK
wherein X is GTG, GGGSATPHFDYIASEVSKGLADLGGSGGSG
GS or GGGSATPHFDYIASEVSKGLADLGGSFGVITADTLEQAIERG
GSGGSGGS,
(o)
(SEQ ID NO: 97-99)
NGSVLILNGPNLNLLGRREPEVYGNTTLEELNASAEAWGAELGLGVVF
NQTNYEGQLIEWVQNASQEGFLAIVLNPGALTHYSYALLDAIRAQPLP
VVEVHLTNLHAREEFRRHSVTAPAARGIVSGFGPLSYKLALVYLAETL
EVGGEGFXNITNLCPFGEVFNATRFASVYAWNRKNITNCVADYSVLYN
SASFSTFKCYGVSPTKLNDLCFTNVSADSFVIRGDEVRQIAPGQTGKI
ADYNYKLPDDFTGCVIAWNSNNLDSKVGGNYNYLYRLFRKSNLKPFER
DISTEIYQAGSTPCNGVEGFNCYFPLQSYGFQPTNGVGYQPYRVVVLS
FELNHTPATVCGPGTKHGKHGK
wherein X is GTG, GGGSATPHFDYIASEVSKGLADLGGSGGSG
GS or GGGSATPHFDYIASEVSKGLADLGGSFGVITADTLEQAIERG
GSGGSGGS,
(p)
(SEQ ID NO: 100-108)
NITNLCPFGEVFNATRFASVYAWNRKNITNCVADYSVLYNSASFSTFK
CYGVSPTNLTDLCFTNVSADSFVIRGDEVRQIAPGQTGKIADYNYKLP
DDFTGCVIAWNSNNLDSKVGGNYNYLYRLFRKSNLKPFERDISTEIYQ
AGSTPCNGVEGFNCYFPLQSYGFQPTNGVGYQPYRVVVLSFELNHTPA
TVCGPGTKHGKHGKX1NGSVLILNGPNLNLLGRREPEVYGNTTLEELN
ASAEAWGAELGLGVVFNQTNYEGQLIEWVQNASQEGFLAIVLNPGALT
HYSYALLDAIRAQPLPVVEVHLTNLHAREEFRRHSVTAPAARGIVSGF
GPLSYKLALVYLAETLEVGGEGFX2NITNLCPFGEVFNATRFASVYAW
NRKNITNCVADYSVLYNSASFSTFKCYGVSPTNLTDLCFTNVSADSFV
IRGDEVRQIAPGQTGKIADYNYKLPDDFTGCVIAWNSNNLDSKVGGNY
NYLYRLFRKSNLKPFERDISTEIYQAGSTPCNGVEGFNCYFPLQSYGF
QPTNGVGYQPYRVVVLSFELNHTPATVCGPGTKHGKHGK
wherein X1 and X2 is independently GTG,
GGGSATPHFDYIASEVSKGLADLGGSGGSGGS or
GGGSATPHFDYIASEVSKGLADLGGSFGVITADTLEQAIERGGSGGSG
GS,
(q)
(SEQ ID NO: 109-117)
NITNLCPFGEVFNATNFSSVYAWNRKNITNCVADYSVLYNSASFSTFK
CYGVSPTKLNDLCFTNVYADSFVIRGDEVRQIAPGQTGKIADYNYKLP
DNFTGCVIAWNSNNLDSKVGGNYNYLYRLFRKSNLKPFERDISTEIYQ
AGSTPCNGVEGENCYFPLQSYGFQPTNGVGYQPYRVVVLSFELNHTPA
TVCGPGTKHGKHGKX1NGSVLILNGPNLNLLGRREPEVYGNTTLEELN
ASAEAWGAELGLGVVFNQTNYEGQLIEWVQNASQEGFLAIVLNPGALT
HYSYALLDAIRAQPLPVVEVHLTNLHAREEFRRHSVTAPAARGIVSGF
GPLSYKLALVYLAETLEVGGEGFX2NITNLCPFGEVFNATNFSSVYAW
NRKNITNCVADYSVLYNSASFSTFKCYGVSPTKLNDLCFTNVYADSFV
IRGDEVRQIAPGQTGKIADYNYKLPDNFTGCVIAWNSNNLDSKVGGNY
NYLYRLFRKSNLKPFERDISTEIYQAGSTPCNGVEGFNCYFPLQSYGF
QPTNGVGYQPYRVVVLSFELNHTPATVCGPGTKHGKHGK
wherein X1 and X2 is independently GTG,
GGGSATPHFDYIASEVSKGLADLGGSGGSGGS or
GGGSATPHFDYIASEVSKGLADLGGSFGVITADTLEQAIERGGSGGSG
GS,
(r)
(SEQ ID NO: 118-126)
NITNLCPFGEVFNATNFSSVYAWNRKNITNCVADYSVLYNSASFSTFK
CYGVSPTNLTDLCFTNVYADSFVIRGDEVRQIAPGQTGKIADYNYKLP
DNFTGCVIAWNSNNLDSKVGGNYNYLYRLFRKSNLKPFERDISTEIYQ
AGSTPCNGVEGENCYFPLQSYGFQPTNGVGYQPYRVVVLSFELNHTPA
TVCGPGTKHGKHGKX1NGSVLILNGPNLNLLGRREPEVYGNTTLEELN
ASAEAWGAELGLGVVFNQTNYEGQLIEWVQNASQEGFLAIVLNPGALT
HYSYALLDAIRAQPLPVVEVHLTNLHAREEFRRHSVTAPAARGIVSGF
GPLSYKLALVYLAETLEVGGEGFX2NITNLCPFGEVFNATNESSVYAW
NRKNITNCVADYSVLYNSASFSTFKCYGVSPTNLTDLCFTNVYADSFV
IRGDEVRQIAPGQTGKIADYNYKLPDNFTGCVIAWNSNNLDSKVGGNY
NYLYRLFRKSNLKPFERDISTEIYQAGSTPCNGVEGFNCYFPLQSYGF
QPTNGVGYQPYRVVVLSFELNHTPATVCGPGTKHGKHGK
wherein X1 and X2 is independently GTG,
GGGSATPHFDYIASEVSKGLADLGGSGGSGGS or
GGGSATPHFDYIASEVSKGLADLGGSFGVITADTLEQAIERGGSGGSG
GS,
(s)
(SEQ ID NO: 127-135)
NITNLCPFGEVFNATRFASVYAWNRKNITNCVADYSVLYNSASFSTFK
CYGVSPTKLNDLCFTNVYADSFVIRGDEVRQIAPGQTGKIADYNYKLP
DNFTGCVIAWNSNNLDSKVGGNYNYLYRLFRKSNLKPFERDISTEIYQ
AGSTPCNGVEGENCYFPLQSYGFQPTNGVGYQPYRVVVLSFELNHTPA
TVCGPGTKHGKHGKX1NGSVLILNGPNLNLLGRREPEVYGNTTLEELN
ASAEAWGAELGLGVVFNQTNYEGQLIEWVQNASQEGFLAIVLNPGALT
HYSYALLDAIRAQPLPVVEVHLTNLHAREEFRRHSVTAPAARGIVSGF
GPLSYKLALVYLAETLEVGGEGFX2NITNLCPFGEVFNATRFASVYAW
NRKNITNCVADYSVLYNSASFSTFKCYGVSPTKLNDLCFTNVYADSFV
IRGDEVRQIAPGQTGKIADYNYKLPDNFTGCVIAWNSNNLDSKVGGNY
NYLYRLFRKSNLKPFERDISTEIYQAGSTPCNGVEGFNCYFPLQSYGF
QPTNGVGYQPYRVVVLSFELNHTPATVCGPGTKHGKHGK
wherein X1 and X2 is independently GTG,
GGGSATPHFDYIASEVSKGLADLGGSGGSGGS or
GGGSATPHFDYIASEVSKGLADLGGSFGVITADTLEQAIERGGSGGSG
GS,
(t)
(SEQ ID NO: 136-144)
NITNLCPFGEVFNATRFASVYAWNRKNITNCVADYSVLYNSASFSTFK
CYGVSPTNLTDLCFTNVYADSFVIRGDEVRQIAPGQTGKIADYNYKLP
DNFTGCVIAWNSNNLDSKVGGNYNYLYRLFRKSNLKPFERDISTEIYQ
AGSTPCNGVEGENCYFPLQSYGFQPTNGVGYQPYRVVVLSFELNHTPA
TVCGPGTKHGKHGKX1NGSVLILNGPNLNLLGRREPEVYGNTTLEELN
ASAEAWGAELGLGVVFNQTNYEGQLIEWVQNASQEGFLAIVLNPGALT
HYSYALLDAIRAQPLPVVEVHLTNLHAREEFRRHSVTAPAARGIVSGF
GPLSYKLALVYLAETLEVGGEGFX2NITNLCPFGEVFNATRFASVYAW
NRKNITNCVADYSVLYNSASFSTFKCYGVSPTNLTDLCFTNVYADSFV
IRGDEVRQIAPGQTGKIADYNYKLPDNFTGCVIAWNSNNLDSKVGGNY
NYLYRLFRKSNLKPFERDISTEIYQAGSTPCNGVEGFNCYFPLQSYGF
QPTNGVGYQPYRVVVLSFELNHTPATVCGPGTKHGKHGK
wherein X1 and X2 is independently GTG,
GGGSATPHFDYIASEVSKGLADLGGSGGSGGS or
GGGSATPHFDYIASEVSKGLADLGGSFGVITADTLEQAIERGGSGGSG
GS,
or
(u)
(SEQ ID NO: 145-153)
NITNLCPFGEVFNATRFASVYAWNRKNITNCVADYSVLYNSASFSTFK
CYGVSPTKLNDLCFTNVSADSFVIRGDEVRQIAPGQTGKIADYNYKLP
DDFTGCVIAWNSNNLDSKVGGNYNYLYRLFRKSNLKPFERDISTEIYQ
AGSTPCNGVEGENCYFPLQSYGFQPTNGVGYQPYRVVVLSFELNHTPA
TVCGPGTKHGKHGKX1NGSVLILNGPNLNLLGRREPEVYGNTTLEELN
ASAEAWGAELGLGVVFNQTNYEGQLIEWVQNASQEGFLAIVLNPGALT
HYSYALLDAIRAQPLPVVEVHLTNLHAREEFRRHSVTAPAARGIVSGF
GPLSYKLALVYLAETLEVGGEGFX2NITNLCPFGEVFNATRFASVYAW
NRKNITNCVADYSVLYNSASFSTFKCYGVSPTKLNDLCFTNVSADSFV
IRGDEVRQIAPGQTGKIADYNYKLPDDFTGCVIAWNSNNLDSKVGGNY
NYLYRLFRKSNLKPFERDISTEIYQAGSTPCNGVEGFNCYFPLQSYGF
QPTNGVGYQPYRVVVLSFELNHTPATVCGPGTKHGKHGK
wherein X1 and X2 is independently GTG,
GGGSATPHFDYIASEVSKGLADLGGSGGSGGS or
GGGSATPHFDYIASEVSKGLADLGGSFGVITADTLEQAIERGGSGGSG
GS, provided X1 or X2 is
GGGSATPHFDYIASEVSKGLADLGGSGGSGGS or
GGGSATPHFDYIASEVSKGLADLGGSFGVITADTLEQAIERGGSGGSG
GS.

In some embodiments, a fusion polypeptide described herein comprising the amino acid sequence of SEQ ID NO: 73-152 or 153.

In some embodiments, a fusion polypeptide described herein comprises a moiety capable of binding to a metal hydroxide adjuvant, e.g., alum. Any methods know to one of skill for linking polypeptides to a metal hydroxide adjuvant, e.g., alum can be used in connection with a fusion polypeptide described herein. See, e.g., US20190358312, which is incorporated herein by reference in its entirety. In some embodiments, the moiety capable of binding to a metal hydroxide adjuvant is positioned within 25 amino acids of the N- or C-terminus of the fusion polypeptide. In some embodiments, the moiety capable of binding to a metal hydroxide adjuvant comprises a peptide comprising phosphoserine repeats, wherein the peptide is conjugated to the fusion polypeptide. In some embodiments, the moiety capable of binding to a metal hydroxide adjuvant is a peptide comprising phosphoserine repeats conjugated to the fusion polypeptide within 25 amino acids of the N- or C-terminus of the fusion polypeptide. In some embodiments, the peptide comprising phosphoserine repeats is conjugated to the fusion polypeptide at one or more cysteine residues. In some embodiments, the cysteine residues are positioned within 25 amino acids of the N- or C-terminus of the fusion polypeptide. In some embodiments, the moiety capable of binding to a metal hydroxide adjuvant comprises cysteine. In some embodiments, the moiety capable of binding to a metal hydroxide adjuvant comprises cysteine positioned within 25 amino acids of the N- or C-terminus of the fusion polypeptide. In some embodiments, the metal hydroxide adjuvant comprises aluminum hydroxide or alum or sodium bis(2-methoxyethoxy)aluminum hydride.

### Polynucleotides

In one aspect, provided herein are isolated polynucleotides encoding pathogen surface glycoprotein receptor binding domains and fusion polypeptides described herein.

In some embodiments, the polynucleotide is DNA.

In some embodiments, the polynucleotide is RNA. In some embodiments, the polynucleotide is mRNA. In some embodiment the RNA, e.g., mRNA comprises modified ribonucleotides.

In some embodiments, an mRNA described herein comprises a coding region encoding a polypeptide described herein, and additionally comprises one or more of a 5′ untranslated region, 3′ untranslated region, 5′ cap, and polyadenylation signal. In some embodiments, an mRNA described herein comprises a coding region encoding a polypeptide described herein, a 5′ untranslated region, a 3′ untranslated region, a 5′ cap, and a polyadenylation signal. In some embodiments, an mRNA described herein comprises modified ribonucleotides. In some embodiments, the mRNA comprises N1-methylpseudouridine or N1-ethylpseudouridine. In some embodiments, the 5′ terminal cap is 7mG(5′)ppp(5′)N1mpNp. See, e.g., US20200261572, US20190351040, and US20190211065, each of which is incorporated herein by reference in its entirety.

In some embodiments, a polynucleotide described herein encodes a fusion polypeptide comprising a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein. In some embodiments, the receptor binding domain comprises the amino acid sequence of SEQ ID NO:33, optionally comprising one or more engineered glycosylation sites, optionally at an amino acid position corresponding to position 346, 357, 360, 370, 381, 386, 394, 428, 444, 458, 468, 481, 518, and/or 522 of SEQ ID NO:51. In some embodiments, the fusion polypeptide further comprises an amino acid sequence that targets the fusion polypeptide to the cell surface. In some embodiments, the fusion polypeptide comprises a GPI anchor signal sequence. In some embodiments, the fusion polypeptide comprises a transmembrane domain. In some embodiments, the transmembrane domain comprises the amino acid sequence of SEQ ID NO:8. In some embodiments, the fusion polypeptide further comprises a self-assembling domain capable of forming a nanoparticle. In some embodiments, the fusion polypeptide comprises a type II 3-Dehydroquinase, ferritin or lumazine synthase. In some embodiments, the fusion polypeptide comprises a type II 3-Dehydroquinase polypeptide comprising one or more engineered glycosylation site. In some embodiments, the fusion polypeptide comprises a Thermus thermophilus type II 3-Dehydroquinase, optionally comprising one or more engineered glycosylation site. In some embodiments, the type II 3-Dehydroquinase polypeptide comprises the amino acid sequence of SEQ ID NO:48. In some embodiments, the type II 3-Dehydroquinase polypeptide comprises one or more engineered glycosylation sites is at an amino acid position corresponding to position 1, 25, 32, 49, and/or 63 of SEQ ID NO:52. In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40) ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42). In some embodiments, the fusion polypeptide further comprises a signal peptide. In some embodiments, the polynucleotide is a DNA. In some embodiments, the polynucleotide is an RNA. In some embodiment the RNA, e.g., mRNA comprises modified ribonucleotides. In some embodiments, the mRNA comprises a coding region encoding a polypeptide described herein, and additionally comprises one or more of a 5′ untranslated region, 3′ untranslated region, 5′ cap, and polyadenylation signal. In some embodiments, the mRNA comprises a coding region encoding a polypeptide described herein, a 5′ untranslated region, a 3′ untranslated region, a 5′ cap, and a polyadenylation signal. In some embodiments, the mRNA comprises modified ribonucleotides. In some embodiments, the mRNA comprises N1-methylpseudouridine or N1-ethylpseudouridine. In some embodiments, the 5′ terminal cap is 7mG(5′)ppp(5′)N1mpNp. See, e.g., US20200261572, US20190351040, and US20190211065, each of which is incorporated herein by reference in its entirety.

In one aspect, provided herein are vectors comprising a polynucleotide described herein.

In one aspect, provided herein are host cells comprising a polynucleotide described herein. In some embodiments, the host cell comprises a CHO cell or a HEK293 cell.

In one aspect, provided herein are recombinant viruses comprising a polynucleotide described herein. In some embodiments, the recombinant virus comprises a DNA virus, a RNA virus, a replicon RNA virus, an alphavirus, a flavivirus, a measles virus, a rhabdovirus, a baculovirus, a poxvirus, a vaccinia virus, an avipox virus, a canarypox a fowlpox virus, a dovepox virus, a modified vaccinia Ankara (MVA), a NYVAC vaccinia virus, an ALVAC canarypox virus, a TROVAC fowlpox virus, an MVA-BN, a herpesvirus, an adenovirus, an adeno-associated virus (AAV), a vesicular stomatitis virus (VSV), a chimeric virus expressing as a surface protein a protein described herein, e.g., a fusion polypeptide described herein. In some embodiments, the recombinant virus comprises an adenovirus or an adeno-associated virus (AAV) comprising a polynucleotide encoding a polypeptide described herein, e.g., a fusion polypeptide described herein.

In some embodiments, provided herein are isolated nucleic acids (e.g., modified mRNAs encoding a polypeptide described herein) comprising a translatable region and a modified nucleoside. In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In one aspect, provided herein are methods of producing a fusion polypeptide described herein, comprising culturing a host cell described herein under suitable conditions to produce the fusion polypeptide. In some embodiments, the host cell comprises a CHO cell or a HEK293 cell.

In one aspect, provided herein are methods of producing an isolated polynucleotide described herein, comprising producing the mRNA through chemical synthesis or in vitro translation.

In one aspect, provided herein is a non-naturally occurring nucleic acid molecule encoding the non-naturally occurring pathogen surface glycoprotein RBD described herein or the non-naturally occurring pathogen or coronavirus surface glycoprotein as described herein.

In one aspect, provided herein is a vector comprising a regulatory element operable in a eukaryotic cell operably linked to a nucleic acid described herein.

In some embodiments, the vector comprises a DNA or DNA plasmid vector.

In some embodiments, the vector comprises an RNA or mRNA vector.

In one aspect, provided herein is a cellular eukaryotic organism, a eukaryotic cell, a mammalian cell, a 293 cell, a VERO cell, a CHO (Chinese Hamster Ovary) cell, a viral vector or a yeast comprising a polynucleotide described herein.

In one aspect, provided herein is a eukaryotic cell, mammalian cell, 293 cell, VERO cell, or CHO cell comprising a vector described herein.

In some embodiments, the vector comprises a viral vector.

In some embodiments, the viral vector comprises a DNA virus, a RNA virus, a replicon RNA virus, an alphavirus, a flavivirus, a measles virus, a rhabdovirus, a baculovirus, a poxvirus, a vaccinia virus, an avipox virus, a canarypox a fowlpox virus, a dovepox virus, a modified vaccinia Ankara (MVA), a NYVAC vaccinia virus, an ALVAC canarypox virus, a TROVAC fowlpox virus, an MVA-BN, a herpesvirus, an adenovirus, an adeno-associated virus (AAV), a vesicular stomatitis virus (VSV), a chimeric virus expressing as a surface protein the non-naturally occurring pathogen or coronavirus surface glycoprotein or the non-naturally occurring pathogen surface glycoprotein RBD.

In some embodiments, the vector comprises a regulatory element operable in a eukaryotic cell operably linked to the nucleic acid molecule. The vector can be any vector as herein discussed, including that the vector can comprise a viral vector, such as AAV, VSV, or a chimeric vector (e.g., VSV or another virus expressing the RBD or surface glycoprotein described herein on the surface of the virus).

Further provided are methods for producing the non-naturally occurring pathogen surface glycoprotein RBD, the non-naturally occurring pathogen or coronavirus surface glycoprotein or fusion polypeptide described herein comprising expressing a non-naturally nucleic acid molecule described herein, or expressing a non-naturally nucleic acid molecule from a vector described herein; and optionally recovering, isolating and/or purifying the non-naturally occurring pathogen surface glycoprotein RBD, the non-naturally occurring pathogen or coronavirus surface glycoprotein, or fusion polypeptide. Advantageously, for a subunit vaccine there is the recovering, isolating and/or purifying.

The nucleotide sequences described herein can be inserted into “vectors.” The term “vector” is widely used and understood by those of skill in the art, and as used herein the term “vector” is used consistent with its meaning to those of skill in the art. For example, the term “vector” is commonly used by those skilled in the art to refer to a vehicle that allows or facilitates the transfer of nucleic acid molecules from one environment to another or that allows or facilitates the manipulation of a nucleic acid molecule.

Any vector that allows expression of the proteins described herein can be used in accordance with the present disclosure. In some embodiments, the proteins described herein can be used in vitro (such as using cell-free expression systems) and/or in cultured cells grown in vitro in order to produce the encoded SARS-CoV-2 proteins, which can then be used for various applications such as in the production of proteinaceous vaccines. For such applications, any vector that allows expression of the proteins in vitro and/or in cultured cells can be used.

For applications where it is desired that the proteins be expressed in vivo, for example when the transgenes are used in DNA or DNA-containing vaccines, any vector that allows for the expression of the proteins described herein and is safe for use in vivo can be used. In some embodiments, the vectors used are safe for use in humans, mammals and/or laboratory animals.

For the proteins described hereinto be expressed, the protein coding sequence should be “operably linked” to regulatory or nucleic acid control sequences that direct transcription and translation of the protein. As used herein, a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” may be any nucleic acid element, such as, but not limited to promoters, enhancers, IRES, introns, and other elements described herein that direct the expression of a nucleic acid sequence or coding sequence that is operably linked thereto. The term “promoter” will be used herein to refer to a group of transcriptional control modules that are clustered around the initiation site for RNA polymerase II and that when operationally linked to the protein coding sequences lead to the expression of the encoded protein. The expression of the transgenes described herein may be under the control of a constitutive promoter or of an inducible promoter, which initiates transcription only when exposed to some particular external stimulus, such as, without limitation, antibiotics such as tetracycline, hormones such as ecdysone, or heavy metals. The promoter can also be specific to a particular cell-type, tissue or organ. Many suitable promoters and enhancers are known in the art, and any such suitable promoter or enhancer can be used for expression of the transgenes. For example, suitable promoters and/or enhancers can be selected from the Eukaryotic Promoter Database (EPDB).

The vectors should typically be chosen such that they contain a suitable gene regulatory region, such as a promoter or enhancer, such that the fusion polypeptides can be expressed.

Any suitable vector may be used depending on the application. For example, plasmids, viral vectors, bacterial vectors, protozoal vectors, insect vectors, baculovirus expression vectors, yeast vectors, mammalian cell vectors, and the like, can be used. Eukaryotic expression vectors are advantageous. Suitable vectors can be selected by the skilled artisan taking into consideration the characteristics of the vector and the requirements for expressing the proteins under the identified circumstances.

Volz describes a recombinant modified vaccinia virus Ankara (MVA) vaccine expressing full-length MERS-CoV spike (S) glycoprotein and immunizing BALB/c mice with either intramuscular or subcutaneous regimens. (Volz et al., Protective Efficacy of Recombinant Modified Vaccinia Virus Ankara Delivering Middle East Respiratory Syndrome Coronavirus Spike Glycoprotein. Journal of Virology July 2015, 89 (16) 8651-8656; DOI: 10.1128/JVI.00614-15), which is incorporated herein by reference in its entirety. Such a vaccine is useful to express fusion polypeptides and immunogens described herein.

Malczyk generated MVs expressing the spike glycoprotein of MERS-CoV in its full-length (MERS-S) or a truncated, soluble variant of MERS-S. (Malczyk et al., A Highly Immunogenic and Protective Middle East Respiratory Syndrome Coronavirus Vaccine Based on a Recombinant Measles Virus Vaccine Platform. Journal of Virology October 2015, 89 (22) 11654-11667; DOI: 10.1128/JVI.01815-15), which is incorporated herein by reference in its entirety. The engineered glycoproteins and fusion polypeptides described herein can be similarly expressed.

Wang generated MERS-CoV VLPs using the baculovirus expression system. Innoculation of Rhesus macaques with MERS-CoV VLPs and Alum adjuvant induced virus-neutralizing antibodies against the RBD. (Wang et al., MERS-CoV virus-like particles produced in insect cells induce specific humoural and cellular imminity in rhesus macaques. Oncotarget. 2017 Feb. 21; 8(8): 12686-12694. Published online 2016 Mar. 30. doi: 10.18632/oncotarget.8475), which is incorporated herein by reference in its entirety. The immunogens and fusion polypeptides provided herein can be similarly be expressed from a baculovirus expression system and used for immunization.

McPherson describes methods for expression, purification, release testing, adjuvant formulation, and animal testing of SARS recombinant spike protein antigen. (McPherson et al., Development of a SARS Coronavirus Vaccine from Recombinant Spike Protein Plus Delta Inulin Adjuvant. All of the vaccine compositions described herein can be produced, formulated, and tested accordingly. In: Vaccine Design Methods and Protocols: Volume 1: Vaccines for Human Diseases, Editors: Sunil Thomas DOI: 10.1007/978-1-4939-3387-7_14), which is incorporated herein by reference in its entirety. All of the vaccine compositions described herein can be produced, formulated, and tested accordingly.

Du reports a recombinant adeno-associated virus (rAAV)-based RBD (RBD-rAAV) vaccine could induce highly potent neutralizing Ab responses in immunized animals. (Du et al., Intranasal Vaccination of Recombinant Adeno-Associated Virus Encoding Receptor-Binding Domain of Severe Acute Respiratory Syndrome Coronavirus (SARS-CoV) Spike Protein Induces Strong Mucosal Immune Responses and Provides Long-Term Protection against SARS-CoV Infection. J Immunol Jan. 15, 2008, 180 (2) 948-956; DOI: 10.4049/jimmunol.180.2.948), which is incorporated herein by reference in its entirety.

The glycoprotein immunogens and fusion polypeptides described herein can likewise be expressed using a recombinant adeno-associated virus system.

Moss (WO2006071250A2, which is incorporated herein by reference in its entirety) reported an attenuated poxvirus carrying a spike (S) polypeptide induces formation of neutralizing antibodies and protectively immunizes animals against a subsequent infection with SARS-CoV. Antiserum collected from animals immunized with the attenuated poxvirus reduced SARS viral replication in infected animals. As also described herein, a secreted, glycosylated S polypeptide including amino acids 14 to 762 of the SARS coronavirus (SARS-CoV) S protein provided complete protection of the upper and lower respiratory tract against SARS infection. Poxviruses replicate entirely in the cytoplasm. They have been used as vaccines since the early 1980's (see, e.g., Panicali, D. et al. Construction of live vaccines by using genetically engineered pox viruses: biological activity of recombinant vaccinia virus expressing influenza virus hemagglutinin, Proc. Natl. Acad. Sci. USA 80:5364-5368, 1983).

Sutter (WO2016116398A1, which is incorporated herein by reference in its entirety) reports development of vaccines and compositions to protect from MERS-CoV infection. Viruses of Modified Vaccinia virus Ankara (MVA) stably containing gene sequences encoding the full-length MERS-CoV proteins S and N were constructed. The recombinant MVA viruses amplified to high titers in chicken embryo fibroblasts and intramuscular vaccination of BALB/c mice with MVA-MERS-SN confirmed the particular immunogenicity of the recombinant N protein. Vaccination raised high levels of serum antibodies that reacted with the authentic N protein of MERS-CoV.

Liu reports a recombinant MERS-CoV vaccine that elicits high-level and lasting neutralizing antibodies in camels. The authors used recombinant nonvirulent Newcastle disease virus (NDV) LaSota strain expressing MERS-CoV S protein. (Liu et al., Newcastle disease virus-based MERS-CoV candidate vaccine elicits high-level and lasting neutralizing antibodies in Bactrian camels. Journal of Integrative Agriculture, October 2017, 16(10,):2264-2273, which is incorporated herein by reference in its entirety). All of the technologies and methods reported above for coronavirus are generally applicable to the engineered RBD-containing glycoproteins and fusion polypeptides described herein.

Wang reviews the development of mRNA-based SARS-CoV-2 vaccines, including non-replicating mRNA vaccines and self-amplifying or replicon RNA vaccines and different delivery methods, such as ex vivo loading of dendritic cell and direct in vivo injection into various anatomical sites. Wang et al., An Evidence Based Perspective on mRNA-SARS-CoV-2 Vaccine Development. Med Sci Monit. 2020; 26: e924700-1-e924700-8. doi: 10.12659/MSM.924700, which is incorporated herein by reference in its entirety.

Inovio has announced positive results from the first-in-human trial of its DNA vaccine against MERS. Modjarrad et al., Safety and immunogenicity of an anti-Middle East respiratory syndrome coronavirus DNA vaccine: A phase 1, open-label, single-arm, dose-escalation trial, The Lancet, Sep. 1, 2019, 19(9)1013-1022; see also Clinical trial NCT0371718: Evaluate the Safety, Tolerability and Immunogenicity Study of GLS-5300 in Healthy Volunteers, which is incorporated herein by reference in its entirety. The compositions, vaccines and methods described herein can be tested and administered by the same procedures.

Generally, mammalian expression systems producing mammalian N-linked glycans are preferred, the goal being to promote neutralizing immune responses involving selected SARS-CoV-2 epitopes while minimize immunogenicity of epitopes that would elicit non-neutralizing antibodies. In certain instances, human expression systems such as HEK293 can be preferred, as animal cells such as CHO, Sp2/0 and NS0 mouse myeloma cells can produce glycoproteins with non-human glycans that can potentially illicit immunogenic responses.

Goh describes the types of host cells used for production of therapeutics, their glycosylation potential and the resultant impact on glycoprotein properties. Goh describes the various complex-type N-linked glycans and commonly used mammalian production cells, including Chinese hamster ovary (CHO) cells, baby hamster kidney (BHK) cells, NS0 myeloma and Sp2/0 hybridoma mouse cell lines, human embryonic kidney cells 293 (HEK293) and HT-1080 human cells. Goh et al., Impact of host cell line choice on glycan profile. Critical Reviews in Biotechnology, 2008, 38:6, 851-867, DOI: 10.1080/07388551.2017.1416577, which is incorporated herein by reference in its entirety. Lalonde reviews mammalian cell lines, including considerations of cell types, cell engineering, gene expression, cell growth and proliferation, protein folding and secretion. Lalonde et al., Therapeutic glycoprotein production in mammalian cells. Journal of Biotechnology, 2017, 251:128; 10.1016/j.jbiotec.2017.04.028, which is incorporated herein by reference in its entirety. The glycoprotein compositions are expressed similarly, taking in to account, for example, variations in types of glycosylation, protein expression and the like.

Hunter reviews strategies for producing recombinant proteins in mammalian cell lines to introduce proper protein folding and post-translational modifications and how to overcome various obstacles that can be encountered. Hunter et al., Optimization of Protein Expression in Mammalian Cells. Current Protocols, February 2019, Volume 95, Issue 1, p. e77. DOI:10.1002/cpps.77, which is incorporated herein by reference in its entirety. Gupta reviews approaches that minimize the glycan heterogeneity for the production of the desired protein with improved glycoforms, including mammalian, insect, and yeast and glycoengineering to produce human-like glycan composition of a recombinant product. Gupta et al., Glycosylation control technologies for recombinant therapeutic proteins. Appl Microbiol Biotechnol 2018, 102, 10457-10468, which is incorporated herein by reference in its entirety.

When the aim is to express the proteins described herein in vivo in a subject, for example in order to generate an immune response against a SARS-CoV-2 antigen and/or protective immunity against SARS-CoV-2, expression vectors that are suitable for expression on that subject, and that are safe for use in vivo, should be chosen. For example, in some embodiments it can be desired to express the proteins described herein in a laboratory animal, such as for pre-clinical testing of the SARS-CoV-2 immunogenic compositions and vaccines described herein. In other embodiments, it will be desirable to express the proteins described herein in human subjects, such as in clinical trials and for actual clinical use of the immunogenic compositions and vaccine described herein. Any vectors that are suitable for such uses can be employed, and it is well within the capabilities of the skilled artisan to select a suitable vector. In some embodiments it can be preferred that the vectors used for these in vivo applications are attenuated to vector from amplifying in the subject. For example, if plasmid vectors are used, preferably they will lack an origin of replication that functions in the subject so as to enhance safety for in vivo use in the subject. If viral vectors are used, preferably they are attenuated or replication-defective in the subject, again, so as to enhance safety for in vivo use in the subject.

In some embodiments described herein viral vectors are used. Viral expression vectors are well known to those skilled in the art and include, for example, viruses such as adenoviruses, adeno-associated viruses (AAV), alphaviruses, herpesviruses, retroviruses and poxviruses, including avipox viruses, attenuated poxviruses, vaccinia viruses, and particularly, the modified vaccinia Ankara virus (MVA; ATCC Accession No. VR-1566). Vesicular stomatitis viruses (VSV) are also contemplated, especially if the VSV G protein is substituted with another protein, such as the fusion polypeptides described herein. Such viruses, when used as expression vectors are innately non-pathogenic in the selected subjects such as humans or have been modified to render them non-pathogenic in the selected subjects. For example, replication-defective adenoviruses and alphaviruses are well known and can be used as gene delivery vectors.

The nucleotide sequences and vectors described herein can be delivered to cells, for example if the aim is to express the SARS-CoV-2 antigens in cells in order to produce and isolate the expressed proteins, such as from cells grown in culture. For expressing the fusion polypeptides in cells any suitable transfection, transformation, or gene delivery methods can be used. Such methods are well known by those skilled in the art, and one of skill in the art would readily be able to select a suitable method depending on the nature of the nucleotide sequences, vectors, and cell types used. For example, transfection, transformation, microinjection, infection, electroporation, lipofection, or liposome-mediated delivery could be used. Expression of the fusion polypeptides can be carried out in any suitable type of host cells, such as bacterial cells, yeast, insect cells, and mammalian cells. The fusion polypeptides described herein can also be expressed using in vitro transcription/translation systems. All of such methods are well known by those skilled in the art, and one of skill in the art would readily be able to select a suitable method depending on the nature of the nucleotide sequences, vectors, and cell types used.

A fusion polypeptide can be chemically synthesized in whole or part using techniques that are well-known in the art (see, e.g., Kochendoerfer, G. G., 2001). Additionally, homologs and derivatives of the polypeptide can be also be synthesized.

Alternatively, methods which are well known to those skilled in the art can be used to construct expression vectors containing nucleic acid molecules that encode the polypeptide or homologs or derivatives thereof under appropriate transcriptional/translational control signals, for expression. These methods include in vitro recombinant DNA techniques, synthetic techniques and in vivo recombination/genetic recombination. See, for example, the techniques described in Maniatis et al., 1989.

Pre-clinical vaccine testing models, in particular the nucleic acid delivery systems, can be adapted to express the fusion polypeptides described herein. J. Yu et al., Science 10.1126/science. abc6284 (2020) reports a naked DNA vaccine immunization in NHPs with various SARS-CoV-2 constructs, including a trimerized RBD, and tests protection against a virus challenge. Corbett et al. (bioRxiv preprint https://doi.org/10.1101/2020.06.11.145920) report development of a Moderna mRNA vaccine called mRNA-1273 that encodes a stabilized SARS-CoV-2 spike which shows elicitation of nAbs and CD8 responses in mice. McKay et al. describe a self-amplifying RNA nanoparticle vaccine to immunize mice with saRNA encoding the SARS-CoV-2 spike protein encapsulated in LNP with doses ranging from 0.01 to 10 μg. (McKay et al., Self-amplifying RNA SARS-CoV-2 lipid nanoparticle vaccine induces equivalent preclinical antibody titers and viral neutralization to recovered COVID-19 patients. bioRxiv preprint doi: 10.1101/2020.04.22.055608 posted Apr. 25, 2020. Each of these publications are incorporated herein by reference in their entirety. The methods described therein can be used to express fusion polypeptides described herein.

Erasmus describes a highly immunogenic vaccine candidate comprised of an RNA replicon (LION) designed to enhance vaccine stability, delivery, and immunogenicity delivery and immunogenicity for intramuscular injection to elicit antibody and T cell responses to SARS-CoV-2. Erasmus et al., Single-dose replicating RNA vaccine induces neutralizing antibodies against SARS-CoV-2 in nonhuman primates. bioRxiv preprint doi: 10.1101/2020.05.28.121640 posted Can 28, 2020, which is incorporated herein by reference in its entirety. The methods described can be used with the fusion polypeptides described herein.

Smith et al (Nature Communicationsl (2020) 11:2601 |https://doi.org/10.1038/s41467-020-16505-0|www.nature.com/naturecommunications) report mouse immunization results with the Inovio DNA vaccine encoding a SARS-CoV-2 spike protein. Quinlan et al (bioRxiv preprint https://doi.org/10.1101/2020.04.10.036418doi) test a subunit vaccine composed of RBD-Fc conjugated to KLH carrier protein tests in rodents. Quinlan et al. show that the prototype elicits neutralizing antibodies and those antibodies do not mediate antibody-dependent enhancement (ADE) under conditions in which Zika virus ADE had previously been observed. Ravichandran et al (bioRxiv preprint https://doi.org/10.1101/2020.05.12.091918) test three different subunit vaccines, including spike ectodomain (S1+S2), S1, and RBD, in rabbits and show that all three subunit vaccines elicit neutralizing Abs. van Doremalen et al (bioRxiv preprint. https://doi.org/10.1101/2020.05.13.093195) report the Oxford vaccine that is being used by AstraZeneca (ChAdOx1 Chimpanzee Adenovirus vector (ChAd)) show that this vaccine provides some protection in NHPs. Each of these publications are incorporated herein by reference in their entirety. The methods described therein can be used to express fusion polypeptides described herein.

Other vaccine modalities and platforms that can be used to express the fusion polypeptides described herein can include the following. Jardine et al (Science Vol 340 10 CAN 2013) report a design of a self-assembling nanoparticle presenting an engineered outer domain from HIV (eOD-GT6 60mer). Sok et al. (Science 30 Sep. 2016 Vol 353 Issue 6307) show that the next generation version of this nanoparticle (eOD-GT8 60mer) induces responses from rare precursors in human-Ig-transgenic mice. Kanekiyo et al. (Nature Immunology|VOL 20|Mar. 2019 1 362-372) test purified protein subunit vaccines that are self-assembling nanoparticles (NPs) presenting RBDs from influenza hemagglutinin that elicit neutralizing responses. Xu et al. describe a DNA vaccine comprising self-assembling nanoparticles comprising lumazine synthase for vaccination with an HIV immunogen and induction of strong humoral responses. (Xu et al., In Vivo Assembly of Nanoparticles Achieved through Synergy of Structure-Based Protein Engineering and Synthetic DNA Generates Enhanced Adaptive Immunity. Adv. Sci. 2020, DOI: 10.1002/advs.201902802. Melo et al. describe an alphavirus RNA replicon for vaccination of subjects with germline-targeting HIV immunogens. (Melo et al., Immunogenicity of RNA Replicons Encoding HIV Env Immunogens Designed for Self-Assembly into Nanoparticles. Molecular Therapy, Vol. 27 No 12, pp. 1-11, December 2019.) Each of these publications are incorporated herein by reference in their entirety. The methods described therein can be used to express fusion polypeptides described herein.

### Immunogenic Compositions

In one aspect, provided herein are immunogenic compositions comprising a pathogen surface glycoprotein receptor binding domain described herein, a fusion polypeptide described herein, a polynucleotide described herein, a vector described herein, or a recombinant virus described herein. In some embodiments, the immunogenic composition comprises a fusion polypeptide described herein. In some embodiments, the immunogenic composition comprises a polynucleotide described herein. In some embodiments, the immunogenic composition comprises a vector described herein. In some embodiments, the immunogenic composition comprises a recombinant virus described herein. In some embodiments, the immunogenic composition further comprises an adjuvant.

In some embodiments, the immunogenic composition comprises a fusion polypeptide described herein. In some embodiments, the fusion polypeptide comprises a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein. In some embodiments, the receptor binding domain comprises the amino acid sequence of SEQ ID NO:33, optionally comprising one or more engineered glycosylation sites, optionally at an amino acid position corresponding to position 346, 357, 360, 370, 381, 386, 394, 428, 444, 458, 468, 481, 518, and/or 522 of SEQ ID NO:51. In some embodiments, the fusion polypeptide further comprises an amino acid sequence that targets the fusion polypeptide to the cell surface. In some embodiments, the fusion polypeptide comprises a GPI anchor signal sequence. In some embodiments, the fusion polypeptide comprises a transmembrane domain. In some embodiments, the transmembrane domain comprises the amino acid sequence of SEQ ID NO:8. In some embodiments, the fusion polypeptide further comprises a self-assembling domain capable of forming a nanoparticle. In some embodiments, the fusion polypeptide comprises a type II 3-Dehydroquinase, ferritin or lumazine synthase. In some embodiments, the fusion polypeptide comprises a type II 3-Dehydroquinase polypeptide comprising one or more engineered glycosylation site. In some embodiments, the fusion polypeptide comprises a Thermus thermophilus type II 3-Dehydroquinase, optionally comprising one or more engineered glycosylation site. In some embodiments, the type II 3-Dehydroquinase polypeptide comprises the amino acid sequence of SEQ ID NO:48. In some embodiments, the type II 3-Dehydroquinase polypeptide comprises one or more engineered glycosylation sites is at an amino acid position corresponding to position 1, 25, 32, 49, and/or 63 of SEQ ID NO:52. In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40), ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42).

In some embodiments, an immunogenic composition described herein comprises a fusion polypeptide comprising an immunogenic polypeptide comprising one or more MHC class II T cell epitope. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40), ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42). In some embodiments, the immunogenic composition comprising the fusion polypeptide with one or more MHC class II T cell epitope is capable of eliciting an increased immune response in a subject compared to the immune response elicited by a reference immunogenic composition comprising a polypeptide without the one or more MHC class II T cell epitope. In some embodiments, the increased immune response is an increased humoral response. In some embodiments, the increased immune response is an increased cellular immune response. In some embodiments, the subject is a mouse or a cynomolgus monkey.

In some embodiments, the immunogenic composition comprises a polynucleotide described herein. In some embodiments, the polynucleotide encodes a fusion polypeptide described herein. In some embodiments, the fusion polypeptide comprises a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein. In some embodiments, the receptor binding domain comprises the amino acid sequence of SEQ ID NO:33, optionally comprising one or more engineered glycosylation sites, optionally at an amino acid position corresponding to position 346, 357, 360, 370, 381, 386, 394, 428, 444, 458, 468, 481, 518, and/or 522 of SEQ ID NO:51. In some embodiments, the fusion polypeptide further comprises an amino acid sequence that targets the fusion polypeptide to the cell surface. In some embodiments, the fusion polypeptide comprises a GPI anchor signal sequence. In some embodiments, the fusion polypeptide comprises a transmembrane domain. In some embodiments, the transmembrane domain comprises the amino acid sequence of SEQ ID NO:8. In some embodiments, the fusion polypeptide further comprises a self-assembling domain capable of forming a nanoparticle. In some embodiments, the fusion polypeptide comprises a type II 3-Dehydroquinase, ferritin or lumazine synthase. In some embodiments, the fusion polypeptide comprises a type II 3-Dehydroquinase polypeptide comprising one or more engineered glycosylation site. In some embodiments, the fusion polypeptide comprises a Thermus thermophilus type II 3-Dehydroquinase, optionally comprising one or more engineered glycosylation site. In some embodiments, the type II 3-Dehydroquinase polypeptide comprises the amino acid sequence of SEQ ID NO:48. In some embodiments, the type II 3-Dehydroquinase polypeptide comprises one or more engineered glycosylation sites is at an amino acid position corresponding to position 1, 25, 32, 49, and/or 63 of SEQ ID NO:52. In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40), ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42). In some embodiments, the fusion polypeptide further comprises a signal peptide. In some embodiments, the polynucleotide is a DNA. In some embodiments, the polynucleotide is an RNA. In some embodiment the RNA, e.g., mRNA comprises modified ribonucleotides. In some embodiments, the mRNA comprises a coding region encoding a polypeptide described herein, and additionally comprises one or more of a 5′ untranslated region, 3′ untranslated region, 5′ cap, and polyadenylation signal. In some embodiments, the mRNA comprises a coding region encoding a polypeptide described herein, a 5′ untranslated region, a 3′ untranslated region, a 5′ cap, and a polyadenylation signal. In some embodiments, the mRNA comprises modified ribonucleotides. In some embodiments, the mRNA comprises N1-methylpseudouridine or N1-ethylpseudouridine. In some embodiments, the 5′ terminal cap is 7mG(5′)ppp(5′)N1mpNp. See, e.g., US20200261572, US20190351040, and US20190211065, each of which is incorporated herein by reference in its entirety.

In some embodiments, an immunogenic composition described herein comprises a polynucleotide encoding a fusion polypeptide comprising an immunogenic polypeptide comprising one or more MHC class II T cell epitope. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40), ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42). In some embodiments, the immunogenic composition comprising the polynucleotide encoding the fusion polypeptide with one or more MHC class II T cell epitope is capable of eliciting an increased immune response in a subject compared to the immune response elicited by a reference immunogenic composition comprising a polynucleotide encoding a polypeptide without the one or more MHC class II T cell epitope. In some embodiments, the polynucleotide is DNA. In some embodiments, the polynucleotide is RNA. In some embodiments, the polynucleotide is mRNA, optionally comprising a modified nucleotide. In some embodiments, the increased immune response is an increased humoral response. In some embodiments, the increased immune response is an increased cellular immune response. In some embodiments, the subject is a mouse or a cynomolgus monkey.

In some embodiments, the immunogenic composition comprises a fusion polypeptide described herein. In some embodiments, the immunogenic composition comprises a polynucleotide described herein. In some embodiments, the immunogenic composition comprises a vector described herein. In some embodiments, the immunogenic composition comprises a recombinant virus described herein.

In some embodiments, an immunogenic composition described herein further comprises an adjuvant. In some embodiments, the adjuvant comprises AS01B, AS03, alum (e.g., Alhydrogel®), Adjuplex™, SMNP, ISCOMs, CpG, and combinations thereof.

Suitable adjuvants are known in the art. Suitable adjuvants include, but are not limited to, mineral salts (e.g., AlK(SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides, such as those described in Chuang, T. H. et al, (2002) J. Leuk. Biol. 71(3): 538-44; Ahmad-Nejad, P. et al (2002) Eur. J. Immunol. 32(7): 1958-68; poly IC or poly AU acids, polyarginine with or without CpG (also known in the art as IC3 1; see Schellack, C. et al (2003) Proceedings of the 34th Annual Meeting of the German Society of Immunology; Lingnau, K. et al (2002) Vaccine 20(29-30): 3498-508), JuvaVax™ (U.S. Pat. No. 6,693,086), certain natural substances (e.g., wax D from Mycobacterium tuberculosis, substances found in Corynebacterium parvum, Bordetella pertussis, or members of the genus Brucella), flagellin (Toll-like receptor 5 ligand; see McSorley, S. J. et al (2002) J. Immunol. 169(7): 3914-9), saponins such as QS21, QS17, and QS7 (U.S. Pat. Nos. 5,057,540; 5,650,398; 6,524,584; 6,645,495), monophosphoryl lipid A, in particular, 3-de-0-acylated monophosphoryl lipid A (3D-MPL), imiquimod (also known in the art as IQM and commercially available as Aldara®; U.S. Pat. Nos. 4,689,338; 5,238,944; Zuber, A K. et al (2004) 22(13-14): 1791-8), and the CCR5 inhibitor CMPD167 (see Veazey, R S. et al (2003) J. Exp. Med. 198: 1551-1562).

Aluminum hydroxide or phosphate (alum) are commonly used at 0.05 to 0.1% solution in phosphate buffered saline. Other adjuvants that can be used, especially with DNA vaccines, are cholera toxin, especially CTA1-DD/ISCOMs (see Mowat, A M. et al (2001) J. Immunol. 167(6): 3398-405), polyphosphazenes (Allcock, H. R. (1998) App. Organometallic Chem. 12(10-11): 659-666; Payne, L. G. et al (1995) Pharm. Biotechnol. 6: 473-93), cytokines such as, but not limited to, IL-2, IL-4, GM-CSF, IL-12, IL-15 IGF-1, IFN-α, IFN-, and IFN-γ (Boyer et al., (2002) J. Liposome Res. 121:137-142; WOOl/095919), immunoregulatory proteins such as CD40L (ADX40; see, for example, WO03/063899), and the CD1a ligand of natural killer cells (also known as CRONY or a-galactosyl ceramide; see Green, T. D. et al, (2003) J. Virol. 77(3): 2046-2055), immunostimulatory fusion proteins such as IL-2 fused to the Fe fragment of immunoglobulins (Barouch et al., Science 290:486-492, 2000) and co-stimulatory molecules B7. 1 and B7.2 (Boyer), all of which can be administered either as proteins or in the form of DNA, on the same expression vectors as those encoding the antigens described herein or on separate expression vectors. In some embodiments, the adjuvant comprises lecithin combined with an acrylic polymer (Adjuplex-LAP), lecithin coated oil droplets in an oil-in-water emulsion (Adjuplex-LE) or lecithin and acrylic polymer in an oil-in-water emulsion (Adjuplex-LAO) (Advanced BioAdjuvants (ABA)). In some embodiments, the adjuvant comprises lecithin. In some embodiments, the adjuvant comprises alum. In some embodiments, the adjuvant comprises saponin, cholesterol and phospholipid. In some embodiments, the adjuvant comprises ISCOMATRIX™. In some embodiments, the adjuvant comprises carbomer homopolymer and lecithin. In some embodiments, the adjuvant comprises Adjuplex™. In some embodiments, the adjuvant comprises poly-ICLC or poly(I:C). In some embodiments, the adjuvant can be a mixture of emulsifier(s), micelle-forming agent, and oil such as that which is commercially available under the name Provax® (IDEC Pharmaceuticals, San Diego, CA). (PEG).

In some embodiments, an immunogenic composition described herein is capable of eliciting neutralizing antibodies against the receptor binding domain (RBD) of SARS-CoV-2 upon administration to a subject. In some embodiments, the subject is a mouse or a cynomolgus monkey. In some embodiments, the subject is a human.

In one aspect, provided herein is an immunogenic or vaccine composition comprising a pharmaceutically or veterinarily acceptable carrier and an effective amount to elicit an immune response, or an effective amount to elicit a protective immune response, of: the non-naturally occurring pathogen surface glycoprotein RBD, or the non-naturally occurring pathogen or coronavirus surface glycoprotein, or the non-naturally nucleic acid molecule, or the vector, described herein.

In some embodiments, the vaccine or immunogenic composition comprises or can be a subunit, polypeptide, DNA, DNA plasmid, mRNA vaccine or immunogenic composition. In some embodiments, the vaccine or immunogenic composition can be lyophilized, or reconstituted lyophilized composition.

In some embodiments, the vaccine or immunogenic composition can be administered, without limitation, orally, nasally, perilingually, sublingually, rectally, subcutaneously, intradermally, or by injection.

In some embodiments, the vaccine or composition can be administered alone, as a single administration; or administered as part of immunization/vaccination regimen such as bi-annually, annually, once or twice or thrice or quarterly or more such as monthly and/or a prime-boost regimen including where prime and boost same or different presentations of antigen (surface glycoprotein). In some embodiments, the vaccine or composition can be administered in two doses, wherein the doses are separated by between about 1 week and about 6 weeks, such as by about 1 week, about 2 weeks, about 3 weeks, about 4 weeks, about 5 weeks or about 6 weeks.

In some embodiments, the vaccine or composition is or can be administered in a regimen wherein regimen comprises administration of one or more immunogenic or vaccine composition against another pathogen, e.g. influenza such as a coronavirus and influenza vaccination or immunization regimen.

In some embodiments, the vaccine or composition is or can be administered as part of a combination vaccine or co-administration or sequential administration with an immunogenic or vaccine composition against another pathogen, e.g. a combination of a coronavirus and influenza.

In some embodiments, the vaccine or immunogenic composition described herein comprises an adjuvant.

In some embodiments, the adjuvant comprises aluminum hydroxide, or alum, or sodium bis(2-methoxyethoxy)aluminum hydride, or an oil-in-water adjuvant, water-in-oil adjuvant, or a carbomer adjuvant.

In some embodiments, the composition comprises the non-naturally occurring pathogen surface glycoprotein RBD, the non-naturally occurring pathogen or coronavirus surface glycoprotein or a fusion polypeptide described herein and the adjuvant comprises aluminum hydroxide, or alum, or sodium bis(2-methoxyethoxy)aluminum hydride.

In some embodiments, the vaccine or immunogenic composition comprising the non-naturally occurring pathogen, coronavirus surface glycoprotein or fusion polypeptide comprising a moiety capable of binding to a metal hydroxide adjuvant; or a moiety capable of binding to a metal hydroxide adjuvant at or near comprising within 25 amino acids of the N- or C-terminus; a moiety capable of binding to a metal hydroxide adjuvant comprising phosphoserine; or a moiety capable of binding to a metal hydroxide adjuvant at or near comprising within 25 amino acids of the N- or C-terminus comprising phosphoserine; or a moiety capable of binding to a metal hydroxide adjuvant comprising cysteine; or a moiety capable of binding to a metal hydroxide adjuvant at or near comprising within 25 amino acids of the N- or C-terminus comprising cysteine; wherein the adjuvant couples with the pathogen, coronavirus surface glycoprotein or fusion polypeptide.

In some embodiments, provided herein is a conjugate comprising the RBD, coronavirus surface glycoprotein, or fusion polypeptide described herein and a moiety capable of binding to a metal hydroxide adjuvant.

In some embodiments, provided herein is an immunogenic composition which comprises the RBD, coronavirus surface glycoprotein, or fusion polypeptide, or conjugate as herein described.

In some embodiments, an immunogenic composition described herein comprises a fusion polypeptide described herein and a nanoparticle. In some embodiments, the nanoparticle is a carbohydrate nanoparticle, a lipid nanoparticle, metallic oxide nanoparticle, or an inorganic nanoparticle.

### Pharmaceutical Compositions

In one aspect, provided herein are pharmaceutical composition comprising a pathogen surface glycoprotein receptor binding domain described herein, a fusion polypeptide described herein, a polynucleotide described herein, a vector described herein, a recombinant virus described herein, or an immunogenic composition described herein and a pharmaceutically acceptable excipient. In some embodiments, the pharmaceutical composition comprises a pathogen surface glycoprotein receptor binding domain described herein. In some embodiments, the pharmaceutical composition comprises a fusion polypeptide described herein. In some embodiments, the pharmaceutical composition comprises a polynucleotide described herein. In some embodiments, the pharmaceutical composition comprises a vector described herein. In some embodiments, the pharmaceutical composition comprises a recombinant virus described herein.

In some embodiments, a pharmaceutical composition described herein comprises a fusion polypeptide described herein. In some embodiments, the fusion polypeptide comprises a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein. In some embodiments, the receptor binding domain comprises the amino acid sequence of SEQ ID NO:33, optionally comprising one or more engineered glycosylation sites, optionally at an amino acid position corresponding to position 346, 357, 360, 370, 381, 386, 394, 428, 444, 458, 468, 481, 518, and/or 522 of SEQ ID NO:51. In some embodiments, the fusion polypeptide further comprises an amino acid sequence that targets the fusion polypeptide to the cell surface. In some embodiments, the fusion polypeptide comprises a GPI anchor signal sequence. In some embodiments, the fusion polypeptide comprises a transmembrane domain. In some embodiments, the transmembrane domain comprises the amino acid sequence of SEQ ID NO:8. In some embodiments, the fusion polypeptide further comprises a self-assembling domain capable of forming a nanoparticle. In some embodiments, the fusion polypeptide comprises a type II 3-Dehydroquinase, ferritin or lumazine synthase. In some embodiments, the fusion polypeptide comprises a type II 3-Dehydroquinase polypeptide comprising one or more engineered glycosylation site. In some embodiments, the fusion polypeptide comprises a Thermus thermophilus type II 3-Dehydroquinase, optionally comprising one or more engineered glycosylation site. In some embodiments, the type II 3-Dehydroquinase polypeptide comprises the amino acid sequence of SEQ ID NO:48. In some embodiments, the type II 3-Dehydroquinase polypeptide comprises one or more engineered glycosylation sites is at an amino acid position corresponding to position 1, 25, 32, 49, and/or 63 of SEQ ID NO:52. In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40), ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42). In some embodiments, the fusion polypeptide further comprises a signal peptide.

In some embodiments, a pharmaceutical composition described herein comprises an mRNA encoding a fusion polypeptide described herein. Pharmaceutical compositions suitable for in vivo delivery of mRNA to a subject, e.g., a human subject are known to one of skill in the art. See, e.g., US20200261572, US20190351040, and US20190211065, each of which is incorporated herein by reference in its entirety. In some embodiments, the fusion polypeptide comprises a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein. In some embodiments, the receptor binding domain comprises the amino acid sequence of SEQ ID NO:33, optionally comprising one or more engineered glycosylation sites, optionally at an amino acid position corresponding to position 346, 357, 360, 370, 381, 386, 394, 428, 444, 458, 468, 481, 518, and/or 522 of SEQ ID NO:51. In some embodiments, the fusion polypeptide further comprises an amino acid sequence that targets the fusion polypeptide to the cell surface. In some embodiments, the fusion polypeptide comprises a GPI anchor signal sequence. In some embodiments, the fusion polypeptide comprises a transmembrane domain. In some embodiments, the transmembrane domain comprises the amino acid sequence of SEQ ID NO:8. In some embodiments, the fusion polypeptide further comprises a self-assembling domain capable of forming a nanoparticle. In some embodiments, the fusion polypeptide comprises a type II 3-Dehydroquinase, ferritin or lumazine synthase. In some embodiments, the fusion polypeptide comprises a type II 3-Dehydroquinase polypeptide comprising one or more engineered glycosylation site. In some embodiments, the fusion polypeptide comprises a Thermus thermophilus type II 3-Dehydroquinase, optionally comprising one or more engineered glycosylation site. In some embodiments, the type II 3-Dehydroquinase polypeptide comprises the amino acid sequence of SEQ ID NO:48. In some embodiments, the type II 3-Dehydroquinase polypeptide comprises one or more engineered glycosylation sites is at an amino acid position corresponding to position 1, 25, 32, 49, and/or 63 of SEQ ID NO:52. In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40) ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42). In some embodiments, the fusion polypeptide further comprises a signal peptide. In some embodiment the mRNA comprises modified ribonucleotides. In some embodiments, the mRNA comprises a coding region encoding a polypeptide described herein, and additionally comprises one or more of a 5′ untranslated region, 3′ untranslated region, 5′ cap, and polyadenylation signal. In some embodiments, the mRNA comprises a coding region encoding a polypeptide described herein, a 5′ untranslated region, a 3′ untranslated region, a 5′ cap, and a polyadenylation signal. In some embodiments, the mRNA comprises modified ribonucleotides. In some embodiments, the mRNA comprises N1-methylpseudouridine or N1-ethylpseudouridine. In some embodiments, the 5′ terminal cap is 7mG(5′)ppp(5′)N1mpNp. See, e.g., US20200261572, US20190351040, and US20190211065, each of which is incorporated herein by reference in its entirety.

The pharmaceutical compositions described herein are prepared in a manner known per se, for example, by means of conventional dissolving, lyophilizing, mixing, granulating or confectioning processes. The pharmaceutical compositions may be formulated according to conventional pharmaceutical practice (see for example, in Remington: The Science and Practice of Pharmacy (22nd ed.), eds. Loyd V. Allen, Jr., 2012, Pharmaceutical Press, Philadelphia, PA, and Encyclopedia of Pharmaceutical Technology, eds. J. Swarbrick and J. C. Boylan, 2013, Marcel Dekker, New York, NY).

In some embodiments, provided herein is a conjugate comprising the RBD, coronavirus surface glycoprotein, or fusion polypeptide described herein and a moiety capable of binding to a metal hydroxide adjuvant.

In some embodiments, provided herein is a pharmaceutical composition which comprises the RBD, coronavirus surface glycoprotein, or fusion polypeptide, or conjugate as herein described.

In some embodiments, a pharmaceutical composition described herein comprises a fusion polypeptide described herein and a nanoparticle. In some embodiments, the nanoparticle is a carbohydrate nanoparticle, a lipid nanoparticle, a metallic oxide nanoparticle, or an inorganic nanoparticle.

### Methods of Use

### Methods of Vaccinating

In one aspect, provided herein are methods of vaccinating a subject comprising administering a therapeutically effective amount of a pathogen surface glycoprotein receptor binding domain described herein, a fusion polypeptide described herein, a polynucleotide described herein, a vector described herein, a recombinant virus described herein, a pharmaceutical composition described herein or an immunogenic composition described herein to the subject. In some embodiments, the method of vaccinating comprises administering a therapeutically effective amount of a pathogen surface glycoprotein receptor binding domain described herein. In some embodiments, the method of vaccinating comprises administering a therapeutically effective amount of a fusion polypeptide described herein. In some embodiments, the method of vaccinating comprises administering a therapeutically effective amount of a polynucleotide described herein. In some embodiments, the method of vaccinating comprises administering a therapeutically effective amount of a vector described herein. In some embodiments, the method of vaccinating comprises administering a therapeutically effective amount of a recombinant virus described herein. In some embodiments, the method of vaccinating comprises administering a therapeutically effective amount of a pharmaceutical composition described herein. In some embodiments, the method of vaccinating comprises administering a therapeutically effective amount of an immunogenic composition described herein. In some embodiments, the subject is a human.

In some embodiments, the method of vaccinating comprises administering a therapeutically effective amount of a fusion polypeptide described herein. In some embodiments, the fusion polypeptide comprises a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein. In some embodiments, the receptor binding domain comprises the amino acid sequence of SEQ ID NO:33, optionally comprising one or more engineered glycosylation sites, optionally at an amino acid position corresponding to position 346, 357, 360, 370, 381, 386, 394, 428, 444, 458, 468, 481, 518, and/or 522 of SEQ ID NO:51. In some embodiments, the fusion polypeptide further comprises an amino acid sequence that targets the fusion polypeptide to the cell surface. In some embodiments, the fusion polypeptide comprises a GPI anchor signal sequence. In some embodiments, the fusion polypeptide comprises a transmembrane domain. In some embodiments, the transmembrane domain comprises the amino acid sequence of SEQ ID NO:8. In some embodiments, the fusion polypeptide further comprises a self-assembling domain capable of forming a nanoparticle. In some embodiments, the fusion polypeptide comprises a type II 3-Dehydroquinase, ferritin or lumazine synthase. In some embodiments, the fusion polypeptide comprises a type II 3-Dehydroquinase polypeptide comprising one or more engineered glycosylation site. In some embodiments, the fusion polypeptide comprises a Thermus thermophilus type II 3-Dehydroquinase, optionally comprising one or more engineered glycosylation site. In some embodiments, the type II 3-Dehydroquinase polypeptide comprises the amino acid sequence of SEQ ID NO:48. In some embodiments, the type II 3-Dehydroquinase polypeptide comprises one or more engineered glycosylation sites is at an amino acid position corresponding to position 1, 25, 32, 49, and/or 63 of SEQ ID NO:52. In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40) ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42). In some embodiments, the fusion polypeptide further comprises a signal peptide. In some embodiments, the subject is a human.

In some embodiments, the method of vaccinating comprises administering a therapeutically effective amount of a polynucleotide described herein, wherein the polynucleotide is an mRNA. In some embodiments, the fusion polypeptide comprises a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein. In some embodiments, the receptor binding domain comprises the amino acid sequence of SEQ ID NO:33, optionally comprising one or more engineered glycosylation sites, optionally at an amino acid position corresponding to position 346, 357, 360, 370, 381, 386, 394, 428, 444, 458, 468, 481, 518, and/or 522 of SEQ ID NO:51. In some embodiments, the fusion polypeptide further comprises an amino acid sequence that targets the fusion polypeptide to the cell surface. In some embodiments, the fusion polypeptide comprises a GPI anchor signal sequence. In some embodiments, the fusion polypeptide comprises a transmembrane domain. In some embodiments, the transmembrane domain comprises the amino acid sequence of SEQ ID NO:8. In some embodiments, the fusion polypeptide further comprises a self-assembling domain capable of forming a nanoparticle. In some embodiments, the fusion polypeptide comprises a type II 3-Dehydroquinase, ferritin or lumazine synthase. In some embodiments, the fusion polypeptide comprises a type II 3-Dehydroquinase polypeptide comprising one or more engineered glycosylation site. In some embodiments, the fusion polypeptide comprises a Thermus thermophilus type II 3-Dehydroquinase, optionally comprising one or more engineered glycosylation site. In some embodiments, the type II 3-Dehydroquinase polypeptide comprises the amino acid sequence of SEQ ID NO:48. In some embodiments, the type II 3-Dehydroquinase polypeptide comprises one or more engineered glycosylation sites is at an amino acid position corresponding to position 1, 25, 32, 49, and/or 63 of SEQ ID NO:52. In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40) ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42). In some embodiments, the fusion polypeptide further comprises a signal peptide. In some embodiment the mRNA comprises modified ribonucleotides. In some embodiments, the mRNA comprises a coding region encoding a polypeptide described herein, and additionally comprises one or more of a 5′ untranslated region, 3′ untranslated region, 5′ cap, and polyadenylation signal. In some embodiments, the mRNA comprises a coding region encoding a polypeptide described herein, a 5′ untranslated region, a 3′ untranslated region, a 5′ cap, and a polyadenylation signal. In some embodiments, the mRNA comprises modified ribonucleotides. In some embodiments, the mRNA comprises N1-methylpseudouridine or N1-ethylpseudouridine. In some embodiments, the 5′ terminal cap is 7mG(5′)ppp(5′)N1mpNp. See, e.g., US20200261572, US20190351040, and US20190211065, each of which is incorporated herein by reference in its entirety. In some embodiments, the subject is a human.

### Methods of Inducing an Immune Response

In one aspect, provided herein are methods of inducing an immune response in a subject comprising administering a therapeutically effective amount of a pathogen surface glycoprotein receptor binding domain described herein, a fusion polypeptide described herein, a polynucleotide described herein, a vector described herein, a recombinant virus described herein, a pharmaceutical composition described herein or an immunogenic composition described herein to the subject. In some embodiments, the method of inducing an immune response comprises administering a therapeutically effective amount of a pathogen surface glycoprotein receptor binding domain described herein. In some embodiments, the method of inducing an immune response comprises administering a therapeutically effective amount of a fusion polypeptide described herein. In some embodiments, the method of inducing an immune response comprises administering a therapeutically effective amount of a polynucleotide described herein. In some embodiments, the method of inducing an immune response comprises administering a therapeutically effective amount of a vector described herein. In some embodiments, the method of inducing an immune response comprises administering a therapeutically effective amount of a recombinant virus described herein. In some embodiments, the method of inducing an immune response comprises administering a therapeutically effective amount of a pharmaceutical composition described herein. In some embodiments, the method of inducing an immune response comprises administering a therapeutically effective amount of an immunogenic composition described herein. In some embodiments, the subject is a human.

In some embodiments, the method of inducing an immune response comprises administering a therapeutically effective amount of a fusion polypeptide described herein. In some embodiments, the fusion polypeptide comprises a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein. In some embodiments, the receptor binding domain comprises the amino acid sequence of SEQ ID NO:33, optionally comprising one or more engineered glycosylation sites, optionally at an amino acid position corresponding to position 346, 357, 360, 370, 381, 386, 394, 428, 444, 458, 468, 481, 518, and/or 522 of SEQ ID NO:51. In some embodiments, the fusion polypeptide further comprises an amino acid sequence that targets the fusion polypeptide to the cell surface. In some embodiments, the fusion polypeptide comprises a GPI anchor signal sequence. In some embodiments, the fusion polypeptide comprises a transmembrane domain. In some embodiments, the transmembrane domain comprises the amino acid sequence of SEQ ID NO:8. In some embodiments, the fusion polypeptide further comprises a self-assembling domain capable of forming a nanoparticle. In some embodiments, the fusion polypeptide comprises a type II 3-Dehydroquinase, ferritin or lumazine synthase. In some embodiments, the fusion polypeptide comprises a type II 3-Dehydroquinase polypeptide comprising one or more engineered glycosylation site. In some embodiments, the fusion polypeptide comprises a Thermus thermophilus type II 3-Dehydroquinase, optionally comprising one or more engineered glycosylation site. In some embodiments, the type II 3-Dehydroquinase polypeptide comprises the amino acid sequence of SEQ ID NO:48. In some embodiments, the type II 3-Dehydroquinase polypeptide comprises one or more engineered glycosylation sites is at an amino acid position corresponding to position 1, 25, 32, 49, and/or 63 of SEQ ID NO:52. In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40) ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42). In some embodiments, the fusion polypeptide further comprises a signal peptide. In some embodiments, the subject is a human.

In some embodiments, the method of inducing an immune response comprises administering a therapeutically effective amount of a polynucleotide described herein, wherein the polynucleotide is an mRNA. In some embodiments, the fusion polypeptide comprises a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein. In some embodiments, the receptor binding domain comprises the amino acid sequence of SEQ ID NO:33, optionally comprising one or more engineered glycosylation sites, optionally at an amino acid position corresponding to position 346, 357, 360, 370, 381, 386, 394, 428, 444, 458, 468, 481, 518, and/or 522 of SEQ ID NO:51. In some embodiments, the fusion polypeptide further comprises an amino acid sequence that targets the fusion polypeptide to the cell surface. In some embodiments, the fusion polypeptide comprises a GPI anchor signal sequence. In some embodiments, the fusion polypeptide comprises a transmembrane domain. In some embodiments, the transmembrane domain comprises the amino acid sequence of SEQ ID NO:8. In some embodiments, the fusion polypeptide further comprises a self-assembling domain capable of forming a nanoparticle. In some embodiments, the fusion polypeptide comprises a type II 3-Dehydroquinase, ferritin or lumazine synthase. In some embodiments, the fusion polypeptide comprises a type II 3-Dehydroquinase polypeptide comprising one or more engineered glycosylation site. In some embodiments, the fusion polypeptide comprises a Thermus thermophilus type II 3-Dehydroquinase, optionally comprising one or more engineered glycosylation site. In some embodiments, the type II 3-Dehydroquinase polypeptide comprises the amino acid sequence of SEQ ID NO:48. In some embodiments, the type II 3-Dehydroquinase polypeptide comprises one or more engineered glycosylation sites is at an amino acid position corresponding to position 1, 25, 32, 49, and/or 63 of SEQ ID NO:52. In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40) ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42). In some embodiments, the fusion polypeptide further comprises a signal peptide. In some embodiment the mRNA comprises modified ribonucleotides. In some embodiments, the mRNA comprises a coding region encoding a polypeptide described herein, and additionally comprises one or more of a 5′ untranslated region, 3′ untranslated region, 5′ cap, and polyadenylation signal. In some embodiments, the mRNA comprises a coding region encoding a polypeptide described herein, a 5′ untranslated region, a 3′ untranslated region, a 5′ cap, and a polyadenylation signal. In some embodiments, the mRNA comprises modified ribonucleotides. In some embodiments, the mRNA comprises N1-methylpseudouridine or N1-ethylpseudouridine. In some embodiments, the 5′ terminal cap is 7mG(5′)ppp(5′)N1mpNp. See, e.g., US20200261572, US20190351040, and US20190211065, each of which is incorporated herein by reference in its entirety. In some embodiments, the subject is a human.

In some embodiments, provided herein are methods of eliciting an immune response in a mammal comprising administering a molecule (e.g., fusion polypeptide or nucleotide encoding the fusion polypeptide) described herein. In some embodiments, the method can result in stimulating a neutralizing antibody (nAb) in the mammal by the method comprising administering the molecule. In some embodiments, the mammal can be any mammal herein discussed, such as a human or a non-human primate, or a mammal having elements of a human immune system, or a mammal is capable of producing human antibodies. In some embodiments, the method includes administering with an adjuvant, such as, for example, alum.

### Methods of Treating a Viral Infection

In one aspect, provided herein are methods of treating a viral infection in a subject comprising administering a therapeutically effective amount of a pathogen surface glycoprotein receptor binding domain described herein, a fusion polypeptide described herein, a polynucleotide described herein, a vector described herein, a recombinant virus described herein, a pharmaceutical composition described herein or an immunogenic composition described herein to the subject. In some embodiments, the method of treating a viral infection comprises administering a therapeutically effective amount of a pathogen surface glycoprotein receptor binding domain described herein. In some embodiments, the method of treating a viral infection comprises administering a therapeutically effective amount of a fusion polypeptide described herein. In some embodiments, the method of treating a viral infection comprises administering a therapeutically effective amount of a polynucleotide described herein. In some embodiments, the method of treating a viral infection comprises administering a therapeutically effective amount of a vector described herein. In some embodiments, the method of treating a viral infection comprises administering a therapeutically effective amount of a recombinant virus described herein. In some embodiments, the method of treating a viral infection comprises administering a therapeutically effective amount of a pharmaceutical composition described herein. In some embodiments, the method of treating a viral infection comprises administering a therapeutically effective amount of an immunogenic composition described herein. In some embodiments, the viral infection is a SARS-CoV-2 infection. In some embodiments, the subject is a human.

In some embodiments, the method of treating a viral infection comprises administering a therapeutically effective amount of a fusion polypeptide described herein. In some embodiments, the fusion polypeptide comprises a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein. In some embodiments, the receptor binding domain comprises the amino acid sequence of SEQ ID NO:33, optionally comprising one or more engineered glycosylation sites, optionally at an amino acid position corresponding to position 346, 357, 360, 370, 381, 386, 394, 428, 444, 458, 468, 481, 518, and/or 522 of SEQ ID NO:51. In some embodiments, the fusion polypeptide further comprises an amino acid sequence that targets the fusion polypeptide to the cell surface. In some embodiments, the fusion polypeptide comprises a GPI anchor signal sequence. In some embodiments, the fusion polypeptide comprises a transmembrane domain. In some embodiments, the transmembrane domain comprises the amino acid sequence of SEQ ID NO:8. In some embodiments, the fusion polypeptide further comprises a self-assembling domain capable of forming a nanoparticle. In some embodiments, the fusion polypeptide comprises a type II 3-Dehydroquinase, ferritin or lumazine synthase. In some embodiments, the fusion polypeptide comprises a type II 3-Dehydroquinase polypeptide comprising one or more engineered glycosylation site. In some embodiments, the fusion polypeptide comprises a Thermus thermophilus type II 3-Dehydroquinase, optionally comprising one or more engineered glycosylation site. In some embodiments, the type II 3-Dehydroquinase polypeptide comprises the amino acid sequence of SEQ ID NO:48. In some embodiments, the type II 3-Dehydroquinase polypeptide comprises one or more engineered glycosylation sites is at an amino acid position corresponding to position 1, 25, 32, 49, and/or 63 of SEQ ID NO:52. In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40) ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42). In some embodiments, the fusion polypeptide further comprises a signal peptide.

In some embodiments, the method of treating a viral infection comprises administering a therapeutically effective amount of a polynucleotide described herein, wherein the polynucleotide is an mRNA. In some embodiments, the fusion polypeptide comprises a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein. In some embodiments, the receptor binding domain comprises the amino acid sequence of SEQ ID NO:33, optionally comprising one or more engineered glycosylation sites, optionally at an amino acid position corresponding to position 346, 357, 360, 370, 381, 386, 394, 428, 444, 458, 468, 481, 518, and/or 522 of SEQ ID NO:51. In some embodiments, the fusion polypeptide further comprises an amino acid sequence that targets the fusion polypeptide to the cell surface. In some embodiments, the fusion polypeptide comprises a GPI anchor signal sequence. In some embodiments, the fusion polypeptide comprises a transmembrane domain. In some embodiments, the transmembrane domain comprises the amino acid sequence of SEQ ID NO:8. In some embodiments, the fusion polypeptide further comprises a self-assembling domain capable of forming a nanoparticle. In some embodiments, the fusion polypeptide comprises a type II 3-Dehydroquinase, ferritin or lumazine synthase. In some embodiments, the fusion polypeptide comprises a type II 3-Dehydroquinase polypeptide comprising one or more engineered glycosylation site. In some embodiments, the fusion polypeptide comprises a Thermus thermophilus type II 3-Dehydroquinase, optionally comprising one or more engineered glycosylation site. In some embodiments, the type II 3-Dehydroquinase polypeptide comprises the amino acid sequence of SEQ ID NO:48. In some embodiments, the type II 3-Dehydroquinase polypeptide comprises one or more engineered glycosylation sites is at an amino acid position corresponding to position 1, 25, 32, 49, and/or 63 of SEQ ID NO:52. In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40) ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42). In some embodiments, the fusion polypeptide further comprises a signal peptide. In some embodiment the mRNA comprises modified ribonucleotides. In some embodiments, the mRNA comprises a coding region encoding a polypeptide described herein, and additionally comprises one or more of a 5′ untranslated region, 3′ untranslated region, 5′ cap, and polyadenylation signal. In some embodiments, the mRNA comprises a coding region encoding a polypeptide described herein, a 5′ untranslated region, a 3′ untranslated region, a 5′ cap, and a polyadenylation signal. In some embodiments, the mRNA comprises modified ribonucleotides. In some embodiments, the mRNA comprises N1-methylpseudouridine or N1-ethylpseudouridine. In some embodiments, the 5′ terminal cap is 7mG(5′)ppp(5′)N1mpNp. See, e.g., US20200261572, US20190351040, and US20190211065, each of which is incorporated herein by reference in its entirety.

### Methods of Preventing or Reducing the Likelihood of a Viral Infection

In one aspect, provided herein are methods of preventing or reducing the likelihood of a viral infection in a subject comprising administering a therapeutically effective amount of a pathogen surface glycoprotein receptor binding domain described herein, a fusion polypeptide described herein, a polynucleotide described herein, a vector described herein, a recombinant virus described herein, a pharmaceutical composition described herein or an immunogenic composition described herein to the subject. In some embodiments, the method of preventing or reducing the likelihood of a viral infection comprises administering a therapeutically effective amount of a pathogen surface glycoprotein receptor binding domain described herein. In some embodiments, the method of preventing or reducing the likelihood of a viral infection comprises administering a therapeutically effective amount of a fusion polypeptide described herein. In some embodiments, the method of preventing or reducing the likelihood of a viral infection comprises administering a therapeutically effective amount of a polynucleotide described herein. In some embodiments, the method of preventing or reducing the likelihood of a viral infection comprises administering a therapeutically effective amount of a vector described herein. In some embodiments, the method of preventing or reducing the likelihood of a viral infection comprises administering a therapeutically effective amount of a recombinant virus described herein. In some embodiments, the method of preventing or reducing the likelihood of a viral infection comprises administering a therapeutically effective amount of a pharmaceutical composition described herein. In some embodiments, the method of preventing or reducing the likelihood of a viral infection comprises administering a therapeutically effective amount of an immunogenic composition described herein. In some embodiments, the viral infection is a SARS-CoV-2 infection. In some embodiments, the subject is a human.

In some embodiments, the method of preventing or reducing the likelihood of a viral infection comprises administering a therapeutically effective amount of a fusion polypeptide described herein. In some embodiments, the fusion polypeptide comprises a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein. In some embodiments, the receptor binding domain comprises the amino acid sequence of SEQ ID NO:33, optionally comprising one or more engineered glycosylation sites, optionally at an amino acid position corresponding to position 346, 357, 360, 370, 381, 386, 394, 428, 444, 458, 468, 481, 518, and/or 522 of SEQ ID NO:51. In some embodiments, the fusion polypeptide further comprises an amino acid sequence that targets the fusion polypeptide to the cell surface. In some embodiments, the fusion polypeptide comprises a GPI anchor signal sequence. In some embodiments, the fusion polypeptide comprises a transmembrane domain. In some embodiments, the transmembrane domain comprises the amino acid sequence of SEQ ID NO:8. In some embodiments, the fusion polypeptide further comprises a self-assembling domain capable of forming a nanoparticle. In some embodiments, the fusion polypeptide comprises a type II 3-Dehydroquinase, ferritin or lumazine synthase. In some embodiments, the fusion polypeptide comprises a type II 3-Dehydroquinase polypeptide comprising one or more engineered glycosylation site. In some embodiments, the fusion polypeptide comprises a Thermus thermophilus type II 3-Dehydroquinase, optionally comprising one or more engineered glycosylation site. In some embodiments, the type II 3-Dehydroquinase polypeptide comprises the amino acid sequence of SEQ ID NO:48. In some embodiments, the type II 3-Dehydroquinase polypeptide comprises one or more engineered glycosylation sites is at an amino acid position corresponding to position 1, 25, 32, 49, and/or 63 of SEQ ID NO:52. In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40) ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42). In some embodiments, the fusion polypeptide further comprises a signal peptide.

In some embodiments, the method of preventing or reducing the likelihood of a viral infection comprises administering a therapeutically effective amount of a polynucleotide described herein, wherein the polynucleotide is an mRNA. In some embodiments, the fusion polypeptide comprises a SARS-CoV-2 S glycoprotein receptor binding domain (RBD) comprising an engineered glycosylation site described herein. In some embodiments, the SARS-CoV-2 RBD comprises one or more engineered glycosylation site at an amino acid position corresponding to position 346, 357, 360, 370, 381, 386, 394, 428, 444, 458, 468, 481, 518, and/or 522 of the SARS-CoV-2 S glycoprotein. In some embodiments, the receptor binding domain comprises the amino acid sequence of SEQ ID NO:33, optionally comprising one or more engineered glycosylation sites, optionally at an amino acid position corresponding to position 346, 357, 360, 370, 381, 386, 394, 428, 444, 458, 468, 481, 518, and/or 522 of SEQ ID NO:51. In some embodiments, the fusion polypeptide further comprises an amino acid sequence that targets the fusion polypeptide to the cell surface. In some embodiments, the fusion polypeptide comprises a GPI anchor signal sequence. In some embodiments, the fusion polypeptide comprises a transmembrane domain. In some embodiments, the transmembrane domain comprises the amino acid sequence of SEQ ID NO:8. In some embodiments, the fusion polypeptide further comprises a self-assembling domain capable of forming a nanoparticle. In some embodiments, the fusion polypeptide comprises a type II 3-Dehydroquinase, ferritin or lumazine synthase. In some embodiments, the fusion polypeptide comprises a type II 3-Dehydroquinase polypeptide comprising one or more engineered glycosylation site. In some embodiments, the fusion polypeptide comprises a Thermus thermophilus type II 3-Dehydroquinase, optionally comprising one or more engineered glycosylation site. In some embodiments, the type II 3-Dehydroquinase polypeptide comprises the amino acid sequence of SEQ ID NO:48. In some embodiments, the type II 3-Dehydroquinase polypeptide comprises one or more engineered glycosylation sites is at an amino acid position corresponding to position 1, 25, 32, 49, and/or 63 of SEQ ID NO:52. In some embodiments, a fusion polypeptide described herein further comprises at least one immunogenic polypeptide comprising one or more MHC class II T cell epitope. In some embodiments, the immunogenic polypeptide comprises one or more MHC class II T cell epitope described herein. In some embodiments, the MHC class II T cell epitope comprises the amino acid sequence of ATPHFDYIASEVSKG (SEQ ID NO:37), FGVITADTLEQAIER (SEQ ID NO:38), FDYIASEVSKGLADL (SEQ ID NO:39), or ATPHFDYIASEVSKGLADL (SEQ ID NO:40). In some embodiments, the immunogenic polypeptide comprises the amino acid sequence of ATPHFDYIASEVSKGLADL (SEQ ID NO:40) ATPHFDYIASEVSKGLADLGGSFGVITADTLEQAIER (SEQ ID NO:41) or ATPHFDYIASEVSKGLADLSLELRKPITFGVITADTLEQAIER (SEQ ID NO:42). In some embodiments, the fusion polypeptide further comprises a signal peptide. In some embodiment the mRNA comprises modified ribonucleotides. In some embodiments, the mRNA comprises a coding region encoding a polypeptide described herein, and additionally comprises one or more of a 5′ untranslated region, 3′ untranslated region, 5′ cap, and polyadenylation signal. In some embodiments, the mRNA comprises a coding region encoding a polypeptide described herein, a 5′ untranslated region, a 3′ untranslated region, a 5′ cap, and a polyadenylation signal. In some embodiments, the mRNA comprises modified ribonucleotides. In some embodiments, the mRNA comprises N1-methylpseudouridine or N1-ethylpseudouridine. In some embodiments, the 5′ terminal cap is 7mG(5′)ppp(5′)N1mpNp. See, e.g., US20200261572, US20190351040, and US20190211065, each of which is incorporated herein by reference in its entirety.

Further provided is a method for eliciting an immune or protective immune response in a mammal, or for eliciting, stimulating or producing an antibody or antibody response in a mammal, or for eliciting, stimulating or producing a neutralizing antibody (nAb) response in a mammal comprising administering an effective amount of the non-naturally occurring pathogen surface glycoprotein RBD, non-naturally occurring pathogen or coronavirus surface glycoprotein, or fusion polypeptide described herein or a vaccine or immunogenic composition described herein; or, expressing in vivo a non-naturally occurring nucleic acid molecule herein described, or, expressing in vivo a non-naturally nucleic acid molecule herein described from a vector herein described. In some embodiments, provided herein is a method wherein the mammal is a human, a non-human primate, a rodent, a chiroptera, or a bat, or a canine, or a dog, or a feline, or a cat, or a porcine, or a pig, or an equine, or a horse, or a bovine, or a cow or bull, or a mink, or a mammal that comprises elements of a human immune system. In some embodiments, the foregoing sentences of this paragraph wherein the mammal is capable of producing human antibodies.

### EXAMPLES

### Example 1. Identification of Lumazine Synthase CD4 T-Cell Epitopes

Intracellular cytokine staining (ICS) assay was used to test 41 overlapping 15-mer peptides spanning the full length of A. aeolicus lumazine synthase (AALS) (MQIYEGKLTAEGLRFGIVASRFNHALVDRLVEGAIDAIVRHGGREEDITLVRVPGSWEIP VAAGELARKEDIDAVIAIGVLIRGATPHFDYIASEVSKGLADLSLELRKPITFGVITADTLE QAIER (SEQ ID NO:42)AGTKHGNKGWEAALSAIEMANLFKSLR) for their ability to increase INFg, IL-2 and/or CD40L expression in human CD4 T cells. The assay was performed substantially as described in Dintwe 2019, Cytometry A 95(7): 722-725 (2019) using 14 frozen human PBMC samples. The PBMC samples were from 14 participants in a clinical trial in which participants were vaccinated twice with the AS01B-adjuvanted protein eOD-GT8 60mer containing A. Aeolicus Lumazine Synthase. In addition to individual 15-mers, a peptide pool comprising all 15-mer peptide was also tested. FIG. 1 shows the results obtained with the panel of peptides. FIG. 2 shows the positive CD-4 T-cell responses by Fisher's Exact Test. Peptides ATPHFDYIASEVSKG (SEQ ID NO:37; LS-22), FGVITADTLEQAIER (SEQ ID NO:38; LS-29) and FDYIASEVSKGLADL (SEQ ID NO:39; LS-23) achieved the highest response rates. 65% (9/14) of vaccine recipients mounted IFNg or IL-2 or CD40L CD4+ T cell responses to the peptide LS-22, 36% (5/14) mounted such responses to the peptide LS-23, and 43% (6/14) mounted responses to the peptide LS-29. Considering combined responses, 71% (10/14) of vaccine recipients responded to LS-22 or LS-23, 86% (12/14) of vaccine recipients responded to LS-22 or LS-29, and 93% (13/14) of vaccine recipients responded to LS-22 or LS-23 or LS-29. These results were independently confirmed in a second set of trial participants who received a higher dose of the same vaccine. 62% of high-dose vaccine recipients tested mounted IFNg or IL-2 or CD40L CD4+ T cell responses to the peptide LS-22, 38% mounted such responses to the peptide LS-23, and 38% mounted responses to the peptide LS-29.

### Example 2. SARS-CoV-2 Specific Immunogens

Membrane-tethered (memRBD) and nanoparticle comprising (RBD-NP) SARS-CoV-2 receptor binding domain (RBD) immunogens optionally comprising supplemental CD4 T help and glycan masking were developed to elicit potent neutralizing antibodies against the receptor binding domain (RBD) of SARS-CoV-2.

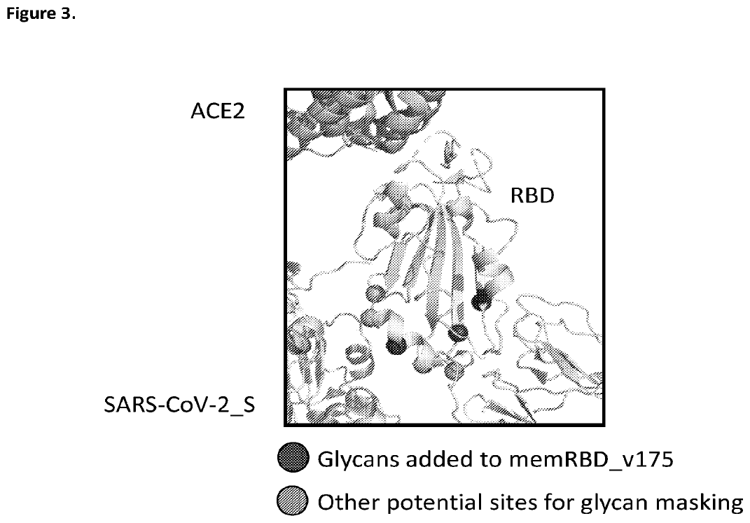

The memRBD and RBD-NP immunogens include optional engineered glycosylation sites in the RBD. Glycosylation sites have been engineered into the RBD at positions so that N-linked glycans attached to those sites during protein expression in mammalian cells will mask the portion of the RBD surface that would be occluded on the SARS-CoV-2 spike trimer. The purpose of the engineered glycosylation sites is to reduce binding or elicitation of non-neutralizing antibodies. The protein structural model in FIG. 3 shows how the glycosylation sites added to RBD are positioned to mask surfaces that would be occluded on the SARS-CoV-2 S trimer when the RBD is in the “up” state required for binding to the ACE2 receptor. These added glycosylation sites also occlude surfaces that would be occluded when the RBD is in the “down” state (not shown). Finally, the glycosylation sites were also positioned so as not to interfere with ACE2 receptor binding. Antibodies targeting surfaces occluded by the glycans on the trimer should be non-neutralizing.

The memRBD immunogens comprise the RBD tethered to a transmembrane domain via a flexible linker (FIG. 4). In some embodiments, RBD C-terminus is linked to the N-terminus of the transmembrane domain (TM) from the G protein of Vesicular Stomatitus Virus (VSV-G). The TM domain serves to anchor the construct in the cell membrane as indicated in the FIG. 4. In some embodiments, the linker region includes CD4 T helper epitopes, such as PADRE or novel MHC class II T cell epitopes from A. aquaticus Lumazine Synthase (AALS) described herein that are known to be immunogenic in humans. In some embodiments, engineered glycosylation sites are added to the RBD to introduce additional glycans to help mask non-neutralizing epitopes, as indicated in the FIG. 3. The amino acid sequence of exemplary memRBD constructs are shown in the Table below.

TABLE 2
memRBD polypeptides. The LS-55 linker includes CD4 helper
epitopes LS-22, LS-23, and LS-29. The LS-37 linker includes
CD4 helper epitopes LS-22 and LS-23. The PADRE-19 linker
includes the PADRE CD4 helper epitope (AKFVAAWTLKAAA
(SEQ ID NO: 36)). The particular signal peptide used here
(MGILPSPGMPALLSLVSLLSVLLMGCVAETG; SEQ ID NO: 5) is not
critical; others can be used. Features are
a) RBD glycosylation; b) linker optionally including
T cell epitope; c) C term.

| Construct | | Sequence with |
|---|---|---|
| Name | Features | SEQ ID NO: 5 signal peptide |
| memRBD_v086 | a) 357 + 386 + | MGILPSPGMPALLSLVSLLSVLLMGCVAETGINLCPFGE |
| (SEQ ID NO: 57) | 394 + 428 + 518 | VFNATRFASVYAWNRKNISNCVADYSVLYNSASFSTFKC |
| | b) PADRE-19 | YGVSPTNLTDLCFTNVSADSFVIRGDEVRQIAPGQTGKI |
| | c) VSV | ADYNYKLPDNFTGCVIAWNSNNLDSKVGGNYNYLYRLFR |
| | | KSNLKPFERDISTEIYQAGSTPCNGVEGFNCYFPLQSYG |
| | | FQPTNGVGYQPYRVVVLSFELNHTPATVCGPGGSAKFVA |
| | | AWTLKAAAGGSKSSIASFFFIIGLIIGLFLVLR (SEQ |
| | | ID NO: 58) |
| memRBD_v144 | a) 357 + 386 + | MGILPSPGMPALLSLVSLLSVLLMGCVAETGINLCPFGE |
| (SEQ ID NO: 59) | 394 + 428 + 518 | VFNATRFASVYAWNRKNISNCVADYSVLYNSASFSTFKC |
| | b) GGS | YGVSPTNLTDLCFTNVSADSFVIRGDEVRQIAPGQTGKI |
| | c) VSV | ADYNYKLPDNFTGCVIAWNSNNLDSKVGGNYNYLYRLFR |
| | | KSNLKPFERDISTEIYQAGSTPCNGVEGFNCYFPLQSYG |
| | | FQPTNGVGYQPYRVVVLSFELNHTPATVCGPGGSGGSGG |
| | | SGGSGGSGGSKSSIASFFFIIGLIIGLFLVLR (SEQ |
| | | ID NO: 60) |
| memRBD_v148 | a) 357 + 386 + | MGILPSPGMPALLSLVSLLSVLLMGCVAETGTNLCPFGE |
| (SEQ ID NO: 61) | 394 + 428 + 518 | VFNATRFASVYAWNRKNISNCVADYSVLYNSASFSTFKC |
| | b) PADRE-31 | YGVSPTNLTDLCFTNVSADSFVIRGDEVRQIAPGQTGKI |
| | c) VSV | ADYNYKLPDNFTGCVIAWNSNNLDSKVGGNYNYLYRLFR |
| | | KSNLKPFERDISTEIYQAGSTPCNGVEGFNCYFPLQSYG |
| | | FQPTNGVGYQPYRVVVLSFELNHTPATVCGPGGSGGSGG |
| | | SAKFVAAWTLKAAAGGSGGSGGSKSSIASFFFIIGLIIG |
| | | LFLVLR (SEQ ID NO: 62) |
| memRBD_v150 | a) 357 + 386 + | MGILPSPGMPALLSLVSLLSVLLMGCVAETGTNLCPFGE |
| (SEQ ID NO: 63) | 394 + 428 + 518 | VFNATRFASVYAWNRKNISNCVADYSVLYNSASFSTFKC |
| | b) LS-37 | YGVSPINLIDLCFTNVSADSFVIRGDEVRQIAPGQTGKI |
| | c) VSV | ADYNYKLPDNFTGCVIAWNSNNLDSKVGGNYNYLYRLFR |
| | | KSNLKPFERDISTEIYQAGSTPCNGVEGFNCYFPLQSYG |
| | | FQPTNGVGYQPYRVVVLSFELNHTPATVCGPGGSGGSGG |
| | | SATPHFDYIASEVSKGLADLGGSGGSGGSKSSIASFFFI |
| | | IGLIIGLFLVLR (SEQ ID NO: 64) |
| memRBD_v151 | a) 357 + 386 + | MGILPSPGMPALLSLVSLLSVLLMGCVAETGTNLCPFGE |
| (SEQ ID NO: 65) | 394 + 428 + 518 | VFNATRFASVYAWNRKNISNCVADYSVLYNSASFSTFKC |
| | b) LS-55 | YGVSPTNLIDLCFTNVSADSFVIRGDEVRQIAPGQTGKI |
| | c) VSV | ADYNYKLPDNFTGCVIAWNSNNLDSKVGGNYNYLYRLFR |
| | | KSNLKPFERDISTEIYQAGSTPCNGVEGFNCYFPLQSYG |
| | | FQPTNGVGYQPYRVVVLSFELNHTPATVCGPGGSGGSGG |
| | | SATPHFDYIASEVSKGLADLGGSFGVITADTLEQAIERG |
| | | GSGGSGGSKSSIASFFFIIGLIIGLFLVLR (SEQ ID |
| | | NO: 66) |
| memRBD_v172 | a) WT | MGILPSPGMPALLSLVSLLSVLLMGCVAETGTNLCPFGE |
| (SEQ ID NO: 67) | b) LS-55 | VFNATRFASVYAWNRKRISNCVADYSVLYNSASFSTFKC |
| | c) VSV | YGVSPTKLNDLCFTNVYADSFVIRGDEVRQIAPGQTGKI |
| | | ADYNYKLPDDFTGCVIAWNSNNLDSKVGGNYNYLYRLFR |
| | | KSNLKPFERDISTEIYQAGSTPCNGVEGFNCYFPLQSYG |
| | | FQPTNGVGYQPYRVVVLSFELLHAPATVCGPGGSGGSGG |
| | | SATPHFDYIASEVSKGLADLGGSFGVITADTLEQAIERG |
| | | GSGGSGGSKSSIASFFFIIGLIIGLFLVLR (SEQ ID |
| | | NO: 68) |
| memRBD_v174 | a) 357 + 386 + | MGILPSPGMPALLSLVSLLSVLLMGCVAETGTNLCPFGE |
| (SEQ ID NO: 69) | 518 | VFNATRFASVYAWNRKNITNCVADYSVLYNSASFSTFKC |
| | b) LS-55 | YGVSPINLIDLCFTNVYADSFVIRGDEVRQIAPGQTGKI |
| | c) VSV | ADYNYKLPDDFIGCVIAWNSNNLDSKVGGNYNYLYRLFR |
| | | KSNLKPFERDISTEIYQAGSTPCNGVEGFNCYFPLQSYG |
| | | FQPTNGVGYQPYRVVVLSFELNHTPATVCGPGGSGGSGG |
| | | SAIPHFDYIASEVSKGLADLGGSFGVITADTLEQAIERG |
| | | GSGGSGGSKSSIASFFFIIGLIIGLFLVLR (SEQ ID |
| | | NO: 70) |
| memRBD_v175 | a) 357 + 386 + | MGILPSPGMPALLSLVSLLSVLLMGCVAETGTNLCPFGE |
| (SEQ ID NO: 71) | 394 + 518 | VFNATRFASVYAWNRKNITNCVADYSVLYNSASFSTFKC |
| | b) LS-55 | YGVSPTNLTDLCFTNVSADSFVIRGDEVRQIAPGQTGKI |
| | c) VSV | ADYNYKLPDDFTGCVIAWNSNNLDSKVGGNYNYLYRLFR |
| | | KSNLKPFERDISTEIYQAGSTPCNGVEGFNCYFPLQSYG |
| | | FQPTNGVGYQPYRVVVLSFELNHTPATVCGPGGSGGSGG |
| | | SATPHFDYIASEVSKGLADLGGSFGVITADTLEQAIERG |
| | | GSGGSGGSKSSIASFFFIIGLIIGLFLVLR (SEQ ID |
| | | NO: 72) |

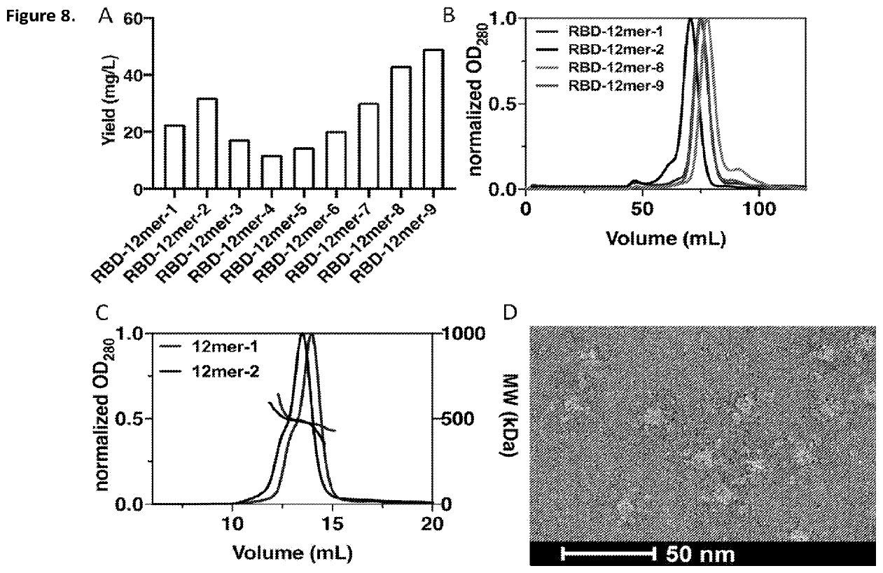

The RBD-NP, (e.g., RBD-12mer and RBD-24mer) immunogens are based on the RBD tethered to a self-assembling domain capable of forming a nanoparticle. In some embodiments, the self-assembling domain the T. thermophilus 3-Dehydroquinase protein, a protein that self-assembles into 12mer nanoparticles (FIG. 7). RBD-NP immunogens include optional supplemental T helper epitopes, for example, the MHC class II T cell epitopes from lumazine synthase described herein that are known to be immunogenic in humans. However, the 151-amino acid T. Thermophilus 3-Dehydroquinase protein very likely contain multiple CD4 T helper epitopes that in combination will be immunogenic in a very large fraction of vaccine recipients. Hence it may not be necessary to add the LS linkers to the RBD-NP (e.g., RBD-12mer or RBD-24mer), meaning that the variants with the GTG linker which show very high expression levels (FIG. 8A) can be used.

In some embodiments, the RBD-12mer and RBD 24-mer constructs comprise an engineered T. thermophilus 3-Dehydroquinase protein with five additional glycosylation sites on its surface at positions 1, 25, 32, 49, and 63 of the dehydroquinase sequence shown in Table 2. In some embodiments, these glycosylation sites promote protein expression and reduce binding or elicitation of antibodies targeting the nanoparticle core.

The amino acid sequence of exemplary RBD-NPs (e.g., RBD-12mer and RBD-24mer constructs) are shown in the Table below. RBD-12mer-2 and -3 include LS-22 and LS-23 CD4 epitopes. RBD-12mer-2 includes LS-22, LS-23, and LS-29. RBD-12mer-7, -8, and -9 include engineered glycans. RBD-12mer-2 and -3 include a His-tag, which is not preferred in a vaccine, but deletion of this tag resulted in reduced expression. RBD-12mer-1 is the same as -2 but replaces this His-tag with HGKHGK (SEQ ID NO:35), which generally pose no problems in a vaccine. “RBD-24mer” nanoparticles comprise RBD subunits fused to both the N- and C-terminus of the T. thermophilus 3-Dehydroquinase protein. The particular signal peptide used here (MGILPSPGMPALLSLVSLLSVLLMGCVAETG; SEQ ID NO: 5) is not critical; others can be used. In some embodiments, an RBD-NP described herein comprises a signal peptide. In some embodiments, the signal peptide comprises SEQ ID NO: 5. In some embodiments, an RBD-NP described herein does not comprise a signal peptide.

TABLE 3
RBD-12 mer fusion polypeptides. X can be either of these linkers: ″GTG″: GTG,
″LS-32″: GGGSATPHFDYIASEVSKGLADLGGSGGSGGS (SEQ ID NO: 49), and ″LS-50″:
GGGSATPHFDYIASEVSKGLADLGGSFGVITADTLEQAIERGGSGGSGGS (SEQ ID
NO: 50). The two linkers in each ″RBD-24 mer″ construct can either be identical
or any combination of two different linkers can be used. Features are a) RBD
glycosylation; b) linker optionally including T cell epitope; c) C term. Linker
sequences of specific RBD-12 mer and RBD-24 mer constructs are identified in
Tables 4 and 5 below, respectively.

| Construct | | |
|---|---|---|
| Name | Features | Sequence |
| RBD-12 mer-1 | a) WT | NGSVLILNGPNLNLLGRREPEVYGNTTLEELNASAEAWG |
| | b) LS-32 | AELGLGVVFNQTNYEGQLIEWVQNASQEGFLAIVLNPGA |
| | c) 2xHGK | LTHYSYALLDAIRAQPLPVVEVHLINLHAREEFRRHSVT |
| | | APAARGIVSGFGPLSYKLALVYLAETLEVGGEGFGGGSA |
| | | TPHFDYIASEVSKGLADLGGSGGSGGSNITNLCPFGEVF |
| | | NATRFASVYAWNRKRISNCVADYSVLYNSASFSTFKCYG |
| | | VSPTKLNDLCFTNVYADSFVIRGDEVRQIAPGQTGKIAD |
| | | YNYKLPDDFTGCVIAWNSNNLDSKVGGNYNYLYRLFRKS |
| | | NLKPFERDISTEIYQAGSTPCNGVEGENCYFPLQSYGFQ |
| | | PTNGVGYQPYRVVVLSFELLHAPATVCGPGTKHGKHGK |
| | | (SEQ ID NO: 73) |
| RBD-12 mer-2 | a) WT | NGSVLILNGPNLNLLGRREPEVYGNTTLEELNASAEAWG |
| | b) LS-50 | AELGLGVVFNQTNYEGQLIEWVQNASQEGFLAIVLNPGA |
| | c) 6xHis | LTHYSYALLDAIRAQPLPVVEVHLINLHAREEFRRHSVT |
| | | APAARGIVSGFGPLSYKLALVYLAETLEVGGEGFGGGSA |
| | | TPHFDYIASEVSKGLADLGGSFGVITADTLEQAIERGGS |
| | | GGSGGSNITNLCPFGEVENATRFASVYAWNRKRISNCVA |
| | | DYSVLYNSASFSTFKCYGVSPTKLNDLCFTNVYADSFVI |
| | | RGDEVRQIAPGQTGKIADYNYKLPDDFTGCVIAWNSNNL |
| | | DSKVGGNYNYLYRLFRKSNLKPFERDISTEIYQAGSTPC |
| | | NGVEGENCYFPLQSYGFQPTNGVGYQPYRVVVLSFELLH |
| | | APATVCGPGTKHHHHHH (SEQ ID NO: 74) |
| RBD-12 mer-3 | a) WT | NGSVLILNGPNLNLLGRREPEVYGNTTLEELNASAEAWG |
| | b) LS-32 | AELGLGVVFNQTNYEGQLIEWVQNASQEGFLAIVLNPGA |
| | c) 6xHis | LTHYSYALLDAIRAQPLPVVEVHLINLHAREEFRRHSVT |
| | | APAARGIVSGFGPLSYKLALVYLAETLEVGGEGFGGGSA |
| | | TPHFDYIASEVSKGLADLGGSGGSGGSNITNLCPFGEVF |
| | | NATRFASVYAWNRKRISNCVADYSVLYNSASFSTFKCYG |
| | | VSPTKLNDLCFTNVYADSFVIRGDEVRQIAPGQTGKIAD |
| | | YNYKLPDDFTGCVIAWNSNNLDSKVGGNYNYLYRLERKS |
| | | NLKPFERDISTEIYQAGSTPCNGVEGENCYFPLQSYGFQ |
| | | PTNGVGYQPYRVVVLSFELLHAPATVCGPGTKHHHHHH |
| | | (SEQ ID NO: 75) |
| RBD-12 mer-4 | a) WT | NGSVLILNGPNLNLLGRREPEVYGNTTLEELNASAEAWG |
| | b) GTG | AELGLGVVFNQTNYEGQLIEWVQNASQEGFLAIVLNPGA |
| | c) 3xGSs | LTHYSYALLDAIRAQPLPVVEVHLINLHAREEFRRHSVT |
| | | APAARGIVSGFGPLSYKLALVYLAETLEVGGEGFGTGTN |
| | | LCPFGEVENATRFASVYAWNRKRISNCVADYSVLYNSAS |
| | | FSTFKCYGVSPTKLNDLCFTNVYADSFVIRGDEVRQIAP |
| | | GQTGKIADYNYKLPDDFTGCVIAWNSNNLDSKVGGNYNY |
| | | LYRLFRKSNLKPFERDISTEIYQAGSTPCNGVEGENCYF |
| | | PLQSYGFQPTNGVGYQPYRVVVLSFELLHAPATVCGPGT |
| | | KGSGSGS (SEQ ID NO: 76) |
| RBD-12 mer-5 | a) WT | NGSVLILNGPNLNLLGRREPEVYGNTTLEELNASAEAWG |
| | b) GTG | AELGLGVVFNQTNYEGQLIEWVQNASQEGFLAIVLNPGA |
| | c) 6xHis | LTHYSYALLDAIRAQPLPVVEVHLINLHAREEFRRHSVT |
| | | APAARGIVSGFGPLSYKLALVYLAETLEVGGEGFGTGTN |
| | | LCPFGEVENATRFASVYAWNRKRISNCVADYSVLYNSAS |
| | | FSTFKCYGVSPTKLNDLCFTNVYADSFVIRGDEVRQIAP |
| | | GQTGKIADYNYKLPDDFTGCVIAWNSNNLDSKVGGNYNY |
| | | LYRLFRKSNLKPFERDISTEIYQAGSTPCNGVEGENCYF |
| | | PLQSYGFQPINGVGYQPYRVVVLSFELLHAPATVCGPGT |
| | | KKKKKKK (SEQ ID NO: 77) |
| RBD-12 mer-6 | a) WT | NGSVLILNGPNLNLLGRREPEVYGNTTLEELNASAEAWG |
| | b) GTG | AELGLGVVFNQTNYEGQLIEWVQNASQEGFLAIVLNPGA |
| | c) 2xHGK | LTHYSYALLDAIRAQPLPVVEVHLINLHAREEFRRHSVT |
| | | APAARGIVSGFGPLSYKLALVYLAETLEVGGEGFGTGTN |
| | | LCPFGEVENATRFASVYAWNRKRISNCVADYSVLYNSAS |
| | | FSTFKCYGVSPTKLNDLCFTNVYADSFVIRGDEVRQIAP |
| | | GQTGKIADYNYKLPDDFTGCVIAWNSNNLDSKVGGNYNY |
| | | LYRLFRKSNLKPFERDISTEIYQAGSTPCNGVEGENCYF |
| | | PLQSYGFQPTNGVGYQPYRVVVLSFELLHAPATVCGPGT |
| | | KHGKHGK (SEQ ID NO: 78) |
| RBD-12 mer-7 | a) 357 + 518 | NGSVLILNGPNLNLLGRREPEVYGNTTLEELNASAEAWG |
| | b) GTG | AELGLGVVFNQTNYEGQLIEWVQNASQEGFLAIVLNPGA |
| | c) 6xHis | LTHYSYALLDAIRAQPLPVVEVHLINLHAREEFRRHSVT |
| | | APAARGIVSGFGPLSYKLALVYLAETLEVGGEGFGTGTN |
| | | LCPFGEVENATRFASVYAWNRKNITNCVADYSVLYNSAS |
| | | FSTFKCYGVSPTKLNDLCFTNVYADSFVIRGDEVRQIAP |
| | | GQTGKIADYNYKLPDDFTGCVIAWNSNNLDSKVGGNYNY |
| | | LYRLFRKSNLKPFERDISTEIYQAGSTPCNGVEGENCYF |
| | | PLQSYGFQPTNGVGYQPYRVVVLSFELNHTPATVCGPGT |
| | | KHHHHHH (SEQ ID NO: 79) |
| RBD-12 mer-8 | a) 346 + 357 + | NGSVLILNGPNLNLLGRREPEVYGNTTLEELNASAEAWG |
| | 428 + 518 | AELGLGVVFNQTNYEGQLIEWVQNASQEGFLAIVLNPGA |
| | b) GTG | LTHYSYALLDAIRAQPLPVVEVHLINLHAREEFRRHSVT |
| | c) 6xHis | APAARGIVSGFGPLSYKLALVYLAETLEVGGEGFGTGTN |
| | | LCPFGEVENATNFSSVYAWNRKNITNCVADYSVLYNSAS |
| | | FSTFKCYGVSPTKLNDLCFTNVYADSFVIRGDEVRQIAP |
| | | GQTGKIADYNYKLPDNFTGCVIAWNSNNLDSKVGGNYNY |
| | | LYRLFRKSNLKPFERDISTEIYQAGSTPCNGVEGENCYF |
| | | PLQSYGFQPTNGVGYQPYRVVVLSFELNHTPATVCGPGT |
| | | KHHHHHH (SEQ ID NO: 80) |
| RBD-12 mer-9 | a) 346 + 357 + | NGSVLILNGPNLNLLGRREPEVYGNTTLEELNASAEAWG |
| | 428 + 518 | AELGLGVVFNQTNYEGQLIEWVQNASQEGFLAIVLNPGA |
| | b) GTG | LTHYSYALLDAIRAQPLPVVEVHLINLHAREEFRRHSVT |
| | c) 6xHis | APAARGIVSGFGPLSYKLALVYLAETLEVGGEGFGTGTN |
| | | LCPFGEVENATNFSSVYAWNRKNITNCVADYSVLYNSAS |
| | | FSTFKCYGVSPTKLNDLCFTNVYADSFVIRGDEVRQIAP |
| | | GQTGKIADYNYKLPDNFTGCVIAWNSNNLDSKVGGNYNY |
| | | LYRLFRKSNLKPFERDISTEIYQAGSTPCNGVEGENCYF |
| | | PLQSYGFQPTNGVGYQPYRVVVLSFELNHTPATVCGPGT |
| | | KHGKHGK (SEQ ID NO: 81) |
| RBD-12 mer-10 | a) 357 + 386 + | NGSVLILNGPNLNLLGRREPEVYGNTTLEELNASAEAWG |
| to -12 | 394 + 518 | AELGLGVVFNQTNYEGQLIEWVQNASQEGFLAIVLNPGA |
| | b) GTG or LS-32 | LTHYSYALLDAIRAQPLPVVEVHLINLHAREEFRRHSVT |
| | or LS-50 | APAARGIVSGFGPLSYKLALVYLAETLEVGGEGFXNITN |
| | c) 2xHGK | LCPFGEVENATRFASVYAWNRKNITNCVADYSVLYNSAS |
| | | FSTFKCYGVSPINLIDLCFTNVSADSFVIRGDEVRQIAP |
| | | GQTGKIADYNYKLPDDFIGCVIAWNSNNLDSKVGGNYNY |
| | | LYRLFRKSNLKPFERDISTEIYQAGSTPCNGVEGENCYF |
| | | PLQSYGFQPTNGVGYQPYRVVVLSFELNHTPATVCGPGT |
| | | KHGKHGK (SEQ ID NO: 82-84) |
| RBD-12 mer-13 | a) 346 + 357 + | NGSVLILNGPNLNLLGRREPEVYGNTTLEELNASAEAWG |
| to -15 | 428 + 518 | AELGLGVVFNQTNYEGQLIEWVQNASQEGFLAIVLNPGA |
| | b) GTG or LS-32 | LTHYSYALLDAIRAQPLPVVEVHLINLHAREEFRRHSVT |
| | or LS-50 | APAARGIVSGFGPLSYKLALVYLAETLEVGGEGFXNITN |
| | c) 2xHGK | LCPFGEVFNATNFSSVYAWNRKNITNCVADYSVLYNSAS |
| | | FSTFKCYGVSPTKLNDLCFTNVYADSFVIRGDEVRQIAP |
| | | GQTGKIADYNYKLPDNFTGCVIAWNSNNLDSKVGGNYNY |
| | | LYRLFRKSNLKPFERDISTEIYQAGSTPCNGVEGENCYF |
| | | PLQSYGFQPINGVGYQPYRVVVLSFELNHTPATVCGPGT |
| | | KHGKHGK (SEQ ID NO: 85-87) |
| RBD-12 mer-16 | a) 346 + 357 + | NGSVLILNGPNLNLLGRREPEVYGNTTLEELNASAEAWG |
| to -18 | 386 + 428 + 518 | AELGLGVVFNQTNYEGQLIEWVQNASQEGFLAIVLNPGA |
| | b) GTG or LS-32 | LTHYSYALLDAIRAQPLPVVEVHLINLHAREEFRRHSVT |
| | or LS-50 | APAARGIVSGFGPLSYKLALVYLAETLEVGGEGFXNITN |
| | c) 2xHGK | LCPFGEVENATNFSSVYAWNRKNITNCVADYSVLYNSAS |
| | | FSTFKCYGVSPINLIDLCFTNVYADSFVIRGDEVRQIAP |
| | | GQTGKIADYNYKLPDNFTGCVIAWNSNNLDSKVGGNYNY |
| | | LYRLFRKSNLKPFERDISTEIYQAGSTPCNGVEGENCYF |
| | | PLQSYGFQPTNGVGYQPYRVVVLSFELNHTPATVCGPGT |
| | | KHGKHGK (SEQ ID NO: 88-90) |
| RBD-12 mer-19 | a) 357 + 428 + | NGSVLILNGPNLNLLGRREPEVYGNTTLEELNASAEAWG |
| to -21 | 518 | AELGLGVVFNQTNYEGQLIEWVQNASQEGFLAIVLNPGA |
| | b) GTG or LS-32 | LTHYSYALLDAIRAQPLPVVEVHLINLHAREEFRRHSVT |
| | or LS-50 | APAARGIVSGFGPLSYKLALVYLAETLEVGGEGFXNITN |
| | c) 2xHGK | LCPFGEVENATRFASVYAWNRKNITNCVADYSVLYNSAS |
| | | FSTFKCYGVSPTKLNDLCFTNVYADSFVIRGDEVRQIAP |
| | | GQTGKIADYNYKLPDNFTGCVIAWNSNNLDSKVGGNYNY |
| | | LYRLFRKSNLKPFERDISTEIYQAGSTPCNGVEGENCYF |
| | | PLQSYGFQPTNGVGYQPYRVVVLSFELNHTPATVCGPGT |
| | | KHGKHGK (SEQ ID NO: 91-93) |
| RBD-12 mer-22 | a) 357 + 386 + | NGSVLILNGPNLNLLGRREPEVYGNTTLEELNASAEAWG |
| to -24 | 428 + 518 | AELGLGVVFNQTNYEGQLIEWVQNASQEGFLAIVLNPGA |
| | b) GTG or LS-32 | LTHYSYALLDAIRAQPLPVVEVHLINLHAREEFRRHSVT |
| | or LS-50 | APAARGIVSGFGPLSYKLALVYLAETLEVGGEGFXNITN |
| | c) 2xHGK | LCPFGEVENATRFASVYAWNRKNITNCVADYSVLYNSAS |
| | | FSTFKCYGVSPTNLTDLCFTNVYADSFVIRGDEVRQIAP |
| | | GQTGKIADYNYKLPDNFTGCVIAWNSNNLDSKVGGNYNY |
| | | LYRLFRKSNLKPFERDISTEIYQAGSTPCNGVEGENCYF |
| | | PLQSYGFQPTNGVGYQPYRVVVLSFELNHTPATVCGPGT |
| | | KHGKHGK (SEQ ID NO: 94-96) |
| RBD-12 mer-25 | a) 357 + 394 + | NGSVLILNGPNLNLLGRREPEVYGNTTLEELNASAEAWG |
| to -27 | 518 | AELGLGVVFNQTNYEGQLIEWVQNASQEGFLAIVLNPGA |
| | b) GTG or LS-32 | LTHYSYALLDAIRAQPLPVVEVHLINLHAREEFRRHSVT |
| | or LS-50 | APAARGIVSGFGPLSYKLALVYLAETLEVGGEGFXNITN |
| | c) 2xHGK | LCPFGEVENATRFASVYAWNRKNITNCVADYSVLYNSAS |
| | | FSTFKCYGVSPTKLNDLCFTNVSADSFVIRGDEVRQIAP |
| | | GQTGKIADYNYKLPDDFIGCVIAWNSNNLDSKVGGNYNY |
| | | LYRLFRKSNLKPFERDISTEIYQAGSTPCNGVEGENCYF |
| | | PLQSYGFQPTNGVGYQPYRVVVLSFELNHTPATVCGPGT |
| | | KHGKHGK (SEQ ID NO: 97-99) |
| RBD-24 mer-1 to | a) 357 + 386 + | NITNLCPFGEVENATRFASVYAWNRKNITNCVADYSVLY |
| -9 | 394 + 518 | NSASFSTFKCYGVSPTNLTDLCFTNVSADSFVIRGDEVR |
| | b) GTG or LS-32 | QIAPGQTGKIADYNYKLPDDFTGCVIAWNSNNLDSKVGG |
| | or LS-50 | NYNYLYRLFRKSNLKPFERDISTEIYQAGSTPCNGVEGF |
| | c) 2xHGK | NCYFPLQSYGFQPTNGVGYQPYRVVVLSFELNHTPATVC |
| | | GPGTKHGKHGKXNGSVLILNGPNLNLLGRREPEVYGNTT |
| | | LEELNASAEAWGAELGLGVVFNQTNYEGQLIEWVQNASQ |
| | | EGFLAIVLNPGALTHYSYALLDAIRAQPLPVVEVHLINL |
| | | HAREEFRRHSVTAPAARGIVSGFGPLSYKLALVYLAETL |
| | | EVGGEGFXNITNLCPFGEVENATRFASVYAWNRKNITNC |
| | | VADYSVLYNSASFSTFKCYGVSPTNLTDLCFTNVSADSF |
| | | VIRGDEVRQIAPGQTGKIADYNYKLPDDFTGCVIAWNSN |
| | | NLDSKVGGNYNYLYRLFRKSNLKPFERDISTEIYQAGST |
| | | PCNGVEGENCYFPLQSYGFQPTNGVGYQPYRVVVLSFEL |
| | | NHTPATVCGPGTKHGKHGK (SEQ ID NO: 100-108) |
| RBD-24 mer-10 | a) 346 + 357 + | NITNLCPFGEVFNATNFSSVYAWNRKNITNCVADYSVLY |
| to - 18 | 428 + 518 | NSASFSTFKCYGVSPTKLNDLCFTNVYADSFVIRGDEVR |
| | b) GTG or LS-32 | QIAPGQTGKIADYNYKLPDNFTGCVIAWNSNNLDSKVGG |
| | or LS-50 | NYNYLYRLFRKSNLKPFERDISTEIYQAGSTPCNGVEGF |
| | c) 2xHGK | NCYFPLQSYGFQPTNGVGYQPYRVVVLSFELNHTPATVC |
| | | GPGTKHGKHGKXNGSVLILNGPNLNLLGRREPEVYGNTT |
| | | LEELNASAEAWGAELGLGVVFNQTNYEGQLIEWVQNASQ |
| | | EGFLAIVLNPGALTHYSYALLDAIRAQPLPVVEVHLINL |
| | | HAREEFRRHSVTAPAARGIVSGFGPLSYKLALVYLAETL |
| | | EVGGEGFXNITNLCPFGEVENATNFSSVYAWNRKNITNC |
| | | VADYSVLYNSASFSTFKCYGVSPTKLNDLCFTNVYADSF |
| | | VIRGDEVRQIAPGQTGKIADYNYKLPDNFTGCVIAWNSN |
| | | NLDSKVGGNYNYLYRLFRKSNLKPFERDISTEIYQAGST |
| | | PCNGVEGENCYFPLQSYGFQPTNGVGYQPYRVVVLSFEL |
| | | NHTPATVCGPGTKHGKHGK (SEQ ID NO: 109-117) |
| RBD-24 mer-19 | a) 346 + 357 + | NITNLCPFGEVFNATNFSSVYAWNRKNITNCVADYSVLY |
| to -27 | 386 + 428 + 518 | NSASFSTFKCYGVSPTNLTDLCFTNVYADSFVIRGDEVR |
| | b) GTG or LS-32 | QIAPGQTGKIADYNYKLPDNFTGCVIAWNSNNLDSKVGG |
| | or LS-50 | NYNYLYRLFRKSNLKPFERDISTEIYQAGSTPCNGVEGF |
| | c) 2xHGK | NCYFPLQSYGFQPTNGVGYQPYRVVVLSFELNHTPATVC |
| | | GPGTKHGKHGKXNGSVLILNGPNLNLLGRREPEVYGNTT |
| | | LEELNASAEAWGAELGLGVVFNQTNYEGQLIEWVQNASQ |
| | | EGFLAIVLNPGALTHYSYALLDAIRAQPLPVVEVHLINL |
| | | HAREEFRRHSVTAPAARGIVSGFGPLSYKLALVYLAETL |
| | | EVGGEGFXNITNLCPFGEVFNATNFSSVYAWNRKNITNC |
| | | VADYSVLYNSASFSTFKCYGVSPTNLTDLCFTNVYADSF |
| | | VIRGDEVRQIAPGQTGKIADYNYKLPDNFTGCVIAWNSN |
| | | NLDSKVGGNYNYLYRLFRKSNLKPFERDISTEIYQAGST |
| | | PCNGVEGENCYFPLQSYGFQPTNGVGYQPYRVVVLSFEL |
| | | NHTPATVCGPGTKHGKHGK (SEQ ID NO: 118-126) |
| RBD-24 mer-28 | a) 357 + 428 + | NITNLCPFGEVENATRFASVYAWNRKNIINCVADYSVLY |
| to -36 | 518 | NSASFSTFKCYGVSPTKLNDLCFTNVYADSFVIRGDEVR |
| | b) GTG or LS-32 | QIAPGQTGKIADYNYKLPDNFTGCVIAWNSNNLDSKVGG |
| | or LS-50 | NYNYLYRLFRKSNLKPFERDISTEIYQAGSTPCNGVEGF |
| | c) 2xHGK | NCYFPLQSYGFQPTNGVGYQPYRVVVLSFELNHTPATVC |
| | | GPGTKHGKHGKXNGSVLILNGPNLNLLGRREPEVYGNTT |
| | | LEELNASAEAWGAELGLGVVFNQTNYEGQLIEWVQNASQ |
| | | EGFLAIVLNPGALTHYSYALLDAIRAQPLPVVEVHLTNL |
| | | HAREEFRRHSVTAPAARGIVSGFGPLSYKLALVYLAETL |
| | | EVGGEGFXNITNLCPFGEVENATRFASVYAWNRKNITNC |
| | | VADYSVLYNSASFSTFKCYGVSPTKLNDLCFTNVYADSF |
| | | VIRGDEVRQIAPGQTGKIADYNYKLPDNFTGCVIAWNSN |
| | | NLDSKVGGNYNYLYRLFRKSNLKPFERDISTEIYQAGST |
| | | PCNGVEGENCYFPLQSYGFQPTNGVGYQPYRVVVLSFEL |
| | | NHTPATVCGPGTKHGKHGK (SEQ ID NO: 127-135) |
| RBD-24 mer-37 | a) 357 + 386 + | NITNLCPFGEVENATRFASVYAWNRKNITNCVADYSVLY |
| to -45 | 428 + 518 | NSASFSTFKCYGVSPTNLTDLCFTNVYADSFVIRGDEVR |
| | b) GTG or LS-32 | QIAPGQTGKIADYNYKLPDNFTGCVIAWNSNNLDSKVGG |
| | or LS-50 | NYNYLYRLFRKSNLKPFERDISTEIYQAGSTPCNGVEGF |
| | c) 2xHGK | NCYFPLQSYGFQPTNGVGYQPYRVVVLSFELNHTPATVC |
| | | GPGTKHGKHGKXNGSVLILNGPNLNLLGRREPEVYGNTT |
| | | LEELNASAEAWGAELGLGVVFNQTNYEGQLIEWVQNASQ |
| | | EGFLAIVLNPGALTHYSYALLDAIRAQPLPVVEVHLINL |
| | | HAREEFRRHSVTAPAARGIVSGFGPLSYKLALVYLAETL |
| | | EVGGEGFXNITNLCPFGEVENATRFASVYAWNRKNITNC |
| | | VADYSVLYNSASFSTFKCYGVSPTNLTDLCFTNVYADSF |
| | | VIRGDEVRQIAPGQTGKIADYNYKLPDNFTGCVIAWNSN |
| | | NLDSKVGGNYNYLYRLFRKSNLKPFERDISTEIYQAGST |
| | | PCNGVEGENCYFPLQSYGFQPTNGVGYQPYRVVVLSFEL |
| | | NHTPATVCGPGTKHGKHGK (SEQ ID NO: 136-144) |
| RBD-24 mer-46 | a) 357 + 394 + | NITNLCPFGEVENATRFASVYAWNRKNITNCVADYSVLY |
| to -54 | 518 | NSASFSTFKCYGVSPTKLNDLCFTNVSADSFVIRGDEVR |
| | b) GTG or LS-32 | QIAPGQTGKIADYNYKLPDDFTGCVIAWNSNNLDSKVGG |
| | or LS-50 | NYNYLYRLFRKSNLKPFERDISTEIYQAGSTPCNGVEGF |
| | c) 2xHGK | NCYFPLQSYGFQPTNGVGYQPYRVVVLSFELNHTPATVC |
| | | GPGTKHGKHGKXNGSVLILNGPNLNLLGRREPEVYGNTT |
| | | LEELNASAEAWGAELGLGVVFNQTNYEGQLIEWVQNASQ |
| | | EGFLAIVLNPGALTHYSYALLDAIRAQPLPVVEVHLINL |
| | | HAREEFRRHSVTAPAARGIVSGFGPLSYKLALVYLAETL |
| | | EVGGEGFXNITNLCPFGEVFNATRFASVYAWNRKNITNC |
| | | VADYSVLYNSASFSTFKCYGVSPTKLNDLCFTNVSADSF |
| | | VIRGDEVRQIAPGQTGKIADYNYKLPDDFTGCVIAWNSN |
| | | NLDSKVGGNYNYLYRLFRKSNLKPFERDISTEIYQAGST |
| | | PCNGVEGENCYFPLQSYGFQPINGVGYQPYRVVVLSFEL |
| | | NHTPATVCGPGTKHGKHGK (SEQ ID NO: 145-153) |

TABLE 4
Linker sequence of RBD-12mer constructs. ″GTG″:
GTG, ″LS-32″: GGGSATPHFDYIASEVSKGLADLGGSGGSGGS
(SEQ ID NO: 49), and ″LS-50″: GGGSATPHFDYIASEVS
KGLADLGGSFGVITADTLEQAIERGGSGGSGGS (SEQ ID
NO: 50).

| Construct Name | Linker | SEQ ID NO |
|---|---|---|
| RBD-12 mer-1 | LS-32 | 73 |
| RBD-12 mer-2 | LS-50 | 74 |
| RBD-12 mer-3 | LS-32 | 75 |
| RBD-12 mer-4 | GTG | 76 |
| RBD-12 mer-5 | GTG | 77 |
| RBD-12 mer-6 | GTG | 78 |
| RBD-12 mer-7 | GTG | 79 |
| RBD-12 mer-8 | GTG | 80 |
| RBD-12 mer-9 | GTG | 81 |
| RBD-12 mer-10 | GTG | 82 |
| RBD-12 mer-11 | LS-32 | 83 |
| RBD-12 mer-12 | LS-50 | 84 |
| RBD-12 mer-13 | GTG | 85 |
| RBD-12 mer-14 | LS-32 | 86 |
| RBD-12 mer-15 | LS-50 | 87 |
| RBD-12 mer-16 | GTG | 88 |
| RBD-12 mer-17 | LS-32 | 89 |
| RBD-12 mer-18 | LS-50 | 90 |
| RBD-12 mer-19 | GTG | 91 |
| RBD-12 mer-20 | LS-32 | 92 |
| RBD-12 mer-21 | LS-50 | 93 |
| RBD-12 mer-22 | GTG | 94 |
| RBD-12 mer-23 | LS-32 | 95 |
| RBD-12 mer-24 | LS-50 | 96 |
| RBD-12 mer-25 | GTG | 97 |
| RBD-12 mer-26 | LS-32 | 98 |
| RBD-12 mer-27 | LS-50 | 99 |

TABLE 5
Linker sequences of RBD-24mer constructs. ″GTG″: GTG, ″LS-32″:
GGGSATPHFDYIASEVSKGLADLGGSGGSGGS (SEQ ID NO: 49), and ″LS-50″:
GGGSATPHFDYIASEVSKGLADLGGSFGVITADTLEQAIERGGSGGSGGS (SEQ ID
NO: 50).

| Construct Name | N terminal linker | C terminal linker | SEQ ID NO |
|---|---|---|---|
| RBD-24 mer-1 | GTG | GTG | 100 |
| RBD-24 mer-2 | GTG | LS-32 | 101 |
| RBD-24 mer-3 | GTG | LS-50 | 102 |
| RBD-24 mer-4 | LS-32 | GTG | 103 |
| RBD-24 mer-5 | LS-32 | LS-32 | 104 |
| RBD-24 mer-6 | LS-32 | LS-50 | 105 |
| RBD-24 mer-7 | LS-50 | GTG | 106 |
| RBD-24 mer-8 | LS-50 | LS-32 | 107 |
| RBD-24 mer-9 | LS-50 | LS-50 | 108 |
| RBD-24 mer-10 | GTG | GTG | 109 |
| RBD-24 mer-11 | GTG | LS-32 | 110 |
| RBD-24 mer-12 | GTG | LS-50 | 111 |
| RBD-24 mer-13 | LS-32 | GTG | 112 |
| RBD-24 mer-14 | LS-32 | LS-32 | 113 |
| RBD-24 mer-15 | LS-32 | LS-50 | 114 |
| RBD-24 mer-16 | LS-50 | GTG | 115 |
| RBD-24 mer-17 | LS-50 | LS-32 | 116 |
| RBD-24 mer-18 | LS-50 | LS-50 | 117 |
| RBD-24 mer-19 | GTG | GTG | 118 |
| RBD-24 mer-20 | GTG | LS-32 | 119 |
| RBD-24 mer-21 | GTG | LS-50 | 120 |
| RBD-24 mer-22 | LS-32 | GTG | 121 |
| RBD-24 mer-23 | LS-32 | LS-32 | 122 |
| RBD-24 mer-24 | LS-32 | LS-50 | 123 |
| RBD-24 mer-25 | LS-50 | GTG | 124 |
| RBD-24 mer-26 | LS-50 | LS-32 | 125 |
| RBD-24 mer-27 | LS-50 | LS-50 | 126 |
| RBD-24 mer-28 | GTG | GTG | 127 |
| RBD-24 mer-29 | GTG | LS-32 | 128 |
| RBD-24 mer-30 | GTG | LS-50 | 129 |
| RBD-24 mer-31 | LS-32 | GTG | 130 |
| RBD-24 mer-32 | LS-32 | LS-32 | 131 |
| RBD-24 mer-33 | LS-32 | LS-50 | 132 |
| RBD-24 mer-34 | LS-50 | GTG | 133 |
| RBD-24 mer-35 | LS-50 | LS-32 | 134 |
| RBD-24 mer-36 | LS-50 | LS-50 | 135 |
| RBD-24 mer-37 | GTG | GTG | 136 |
| RBD-24 mer-38 | GTG | LS-32 | 137 |
| RBD-24 mer-39 | GTG | LS-50 | 138 |
| RBD-24 mer-40 | LS-32 | GTG | 139 |
| RBD-24 mer-41 | LS-32 | LS-32 | 140 |
| RBD-24 mer-42 | LS-32 | LS-50 | 141 |
| RBD-24 mer-43 | LS-50 | GTG | 142 |
| RBD-24 mer-44 | LS-50 | LS-32 | 143 |
| RBD-24 mer-45 | LS-50 | LS-50 | 144 |
| RBD-24 mer-46 | GTG | GTG | 145 |
| RBD-24 mer-47 | GTG | LS-32 | 146 |
| RBD-24 mer-48 | GTG | LS-50 | 147 |
| RBD-24 mer-49 | LS-32 | GTG | 148 |
| RBD-24 mer-50 | LS-32 | LS-32 | 149 |
| RBD-24 mer-51 | LS-32 | LS-50 | 150 |
| RBD-24 mer-52 | LS-50 | GTG | 151 |
| RBD-24 mer-53 | LS-50 | LS-32 | 152 |
| RBD-24 mer-54 | LS-50 | LS-50 | 153 |

memRBD and RBD-NP achieve good expression and antigenicity, and good assembly in the case of RBD-NP, when incorporating either (i) LS-22 and LS-23 or (ii) LS-22, LS-23, and LS-29, into the respective linker/tether regions. See, FIGS. 5 and 6 for memRBD constructs; and FIGS. 8-10 for RBD-NP.

Inclusion of the MHC class II CD4 T cell epitopes that are broadly immunogenic in diverse humans will help the memRBD and RBD-NP vaccines elicit CD4 T cell-dependent antibody responses in a larger fraction of vaccine recipients than they would otherwise, and also will improve the elicitation of potent and durable antibody responses.

Without optional engineered RBD glycosylation sites, the memRBD and RBD-NP constructs exhibit excellent expression levels and binding to neutralizing antibodies, as well as binding to non-neutralizing antibodies. With optional engineered RBD glycosylation sites, the memRBD and RBD-NP constructs also exhibit excellent expression levels and binding to neutralizing antibodies but have reduced or undetectable binding to non-neutralizing antibodies.

FIG. 5 shows the cell surface antigenicity of memRBD variants with different linker regions. Cells were transfected with negative control membrane-bound protein (Neg), full-length stabilized SARS2 S protein (SARS2_S_2P) or glycosylated and membrane-tethered RBD (memRBD_v144, v086, v148, v150, vl51) tethered to a VSV-G transmembrane-domain via the indicated linker (GGS, PADRE-19, PADRE-31, LS-37, or LS-55). All glycosylated memRBD constructs in this experiment had WT glycans plus engineered glycans at positions 357, 386, 394, 428, and 518. Two days after transfection, cells were stained with the indicated mAbs, labelled with Alexa647-conjugated antihuman IgG, and analyzed by flow cytometry. Median fluorescence intensities of transfected single live cells were calculated in FlowJo. All glycosylated memRBD constructs completely abolished binding of both non-neutralizing antibodies tested (CC12.19 and CR3022) but were bound by neutralizing mAb CC6.29 to approximately the same level as full-length S protein.

FIG. 6 shows antigenic profile of memRBD variants with different glycan-masking. Cells were transfected with negative control membrane-bound protein (Neg), full-length stabilized SARS2 S protein (SARS2_S_2P) or glycosylated and membrane-tethered RBD (memRBD_v172, v175, v174, vl51) tethered to a VSV-G transmembrane-domain via a LS-55 linker. Glycosylation sites present on each protein are indicated, with WT indicating wild-type glycosylation sites and 357, 386, 394, 428, and 518 indicating the position of an engineered glycosylation site. Two days after transfection, cells were stained with the indicated mAbs, labelled with Alexa647-conjugated antihuman IgG, and analyzed by flow cytometry. Median fluorescence intensities of transfected single cells were calculated in FlowJo. These data reveal several key observations. First, memRBD_v172 (WT glycosylation sites only) and memRBD_v175 (WT and engineered glycosylation sites) showed stronger binding to most NAbs compared to 2P-stabilized S protein. This suggests that, in comparison with the full-length S protein, these two memRBD constructs either express at higher level on the cell surface or show higher exposure of neutralizing RBD epitopes, or both. Second, both the S protein and the memRBD variant lacking engineered glycans (v172) bind to non-neutralizing antibodies, with memRBD_v172 binding more strongly than S_2P to RBD non-NAbs in accord with higher expression and/or higher RBD epitope exposure as noted above, and S_2P but not memRBD_v172 showing binding to the non-RBD non-NAb CC12.21 in accord with the fact that memRBD constructs do not contain non-RBD epitopes present on S protein. Third, while the memRBD construct lacking engineered glycans (v172) bound strongly to RBD non-NAbs, all memRBD constructs with engineered glycans (v175, v174, and v151) showed no or minimal detectable binding to RBD non-NAbs. This third observation suggest that the engineered glycosylation sites are occupied and the additional glycans are capable of masking the non-neutralizing epitopes targeted by the non-NAbs tested. Overall, these data indicate that memRBD_v172 and memRBD_v175 are promising vaccine candidates in terms of cell-surface expression and antigenicity. The fact that v172 and v175 both include CD4 T helper epitopes from lumazine synthase that we have separately found to be immunogenic in humans adds to their promise as vaccine candidates.

FIG. 8 shows the expression yield and assembly and homogeneity of RBD-12mers. RBD-12mers were expressed in freestyle 293F cells and purified by lectin-affinity chromatography. Yields were determined by OD₂₈₀ measurement. FIG. 8A. Yields were >10 mg/L for RBD-12mer-3, -4 and -5, >20 mg/L for RBD-12mer-1 and -5, >30 mg/L for RBD-12mer-2 and -7, and >40 mg/L for RBD-12mer-8 and -9. Preparative SEC purification revealed a single predominant peak in each case for RBD-12mer-1, -2, -8, and -9. FIG. 8B. SEC-MALS analysis was performed on lectin-purified samples to characterize the uniformity of assembly and to measure molecular weights of particles in solution. FIG. 8C. The measured molecular weights across the peak are consistent with theoretical molecular weights of fully assembled particles. Overall the SEC-MALS data show that the nanoparticles assemble with high fidelity and homogeneity, with no significant population of partially assembled (lower MW) particles, and with a small shoulder on the left of the peaks indicating only a small amount of slightly higher MW assemblies. Negative stain electron tomography of RBD-12mer-1 shows compact nanoparticle cores surrounded by several smaller RBD subunits. FIG. 8D.

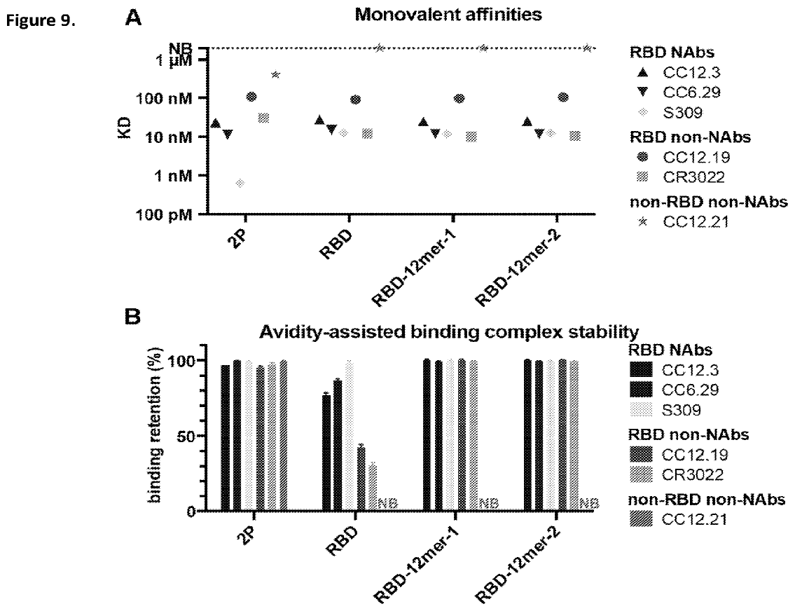

FIG. 9 shows Bio-Layer Interferometry (BLI) analysis of antigenicity of RBD-12mer-1 and RBD-12mer-2. Comparison of monovalent binding affinities of SARS-CoV-2-specific Fabs binding to RBD monomer, RBD-12mers, and stabilized SARS-CoV-2 S protein trimer (2P). FIG. 9A. RBD-12mers and 2P trimer were captured onto Streptavidin sensor tips pre-coated with biotinylated Galanthus Nivalis lectin. Titration series of the indicated Fabs were passed over the constructs for 180 seconds followed by 300 seconds of buffer. His-tagged monomeric RBD (RBD) was included as a control and captured using Anti-His Biosensors. Data from a reference sensor were subtracted from each curve; Y-axes were aligned to the baseline phase; and data were fit to a 1:1 Langmuir binding model in the Octet Data Analysis software (version 11.0.2.3, ForteBio). The data show that the RBD nanoparticles have very similar monovalent affinities as the RBD monomer and the 2P trimer, demonstrating that the RBD domains on the nanoparticle are properly folded and the relevant RBD epitopes are well exposed. As expected, the one non-RBD-specific Fab tested (CC12.21) binds to 2P (which contains RBD and non-RBD epitopes) but not to RBD monomer or RBD-12mers. Comparison of binding avidities of SARS-CoV-2-specific IgG antibodies binding to RBD monomer, RBD-12mers, and stabilized SARS-CoV-2 S protein trimer (2P). FIG. 9B. The indicated antibodies were captured onto Protein A biosensors. The same proteins as in FIG. 9A were then passed over the IgGs at a protomer concentration of 100 nM, followed by washing with buffer. The binding signal after 120 seconds was divided by the signal at the beginning of the wash. NB: No binding. The results demonstrate increased avidity of the RBD-12mers over the RBD monomer.

FIG. 10 compares antigenicity of different RBD-12mers by Bio-Layer Interferometry (BLI). Glycosylation sites and C-terminal sequence for each RBD-12mer is indicated. All RBD-12mers in this figure used a GTG linker and included five engineered glycosylation sites on the 3-Dehydroquinase core nanoparticle. The indicated antibodies were captured onto Protein A biosensors. RBD-12mers, stabilized SARS-CoV-2 spike protein (2P) or monomeric RBD were expressed in freestyle 293F cells, and clarified supernatants were passed over the IgGs for 180 seconds. Raw binding signals after 180 seconds were plotted in Graphpad Prism. The binding data demonstrate that the particles with additional glycans on the RBD (RBD-12mer-7 through RBD-12mer-9) only show minimal binding to non-neutralizing antibody CC12.19.

Both memRBD and RBD-NPimmunogens provide for multivalent display of RBD. The positive expression and antigenicity of this platform, combined with their multivalency, indicates that both have potential to elicit a focused neutralizing antibody response to the RBD. As the RBD contains a dominant fraction of the neutralization epitopes on the SARS-CoV-2 S protein, this RBD-focused response will allow for protective responses from lower vaccine doses compared to an S protein vaccine, reducing the cost of each dose and increasing the number of people that can be vaccinated from one batch of vaccine.

The addition of engineered glycans to mask non-neutralizing epitopes will reduce the non-neutralizing response and will therefore further enhance the neutralizing response. This potentially further enhanced response will allow for further dose sparing and expansion of the number of vaccinated people from a fixed quantity of vaccine.

In some embodiments, memRBD immunogens will be delivered by nucleic acid or viral vector approaches.

In some embodiments, delivery of RBD-NP constructs will be by nucleic acid or viral vector approaches, or by traditional purified protein approaches. The high fidelity assembly of the RBD-NP, evidenced by the >90% particle formation from a lectin-purified sample (FIG. 8), indicates that the RBD-NP is a promising platform for nucleic or viral vector approaches where purification of the expressed protein is not possible.

The relatively small number of amino acids included in the memRBD (305aa with LS-55 linker) and RBD-NP (394 for GTG linker; 438aa for LS-55 linker) compared to the full-length spike protein (˜1273aa) provides other advantages: it further contributes to dose sparing for nucleic acid delivery, and, in the context of viral vector delivery the smaller size of the insert reduces the burden on viral fitness.

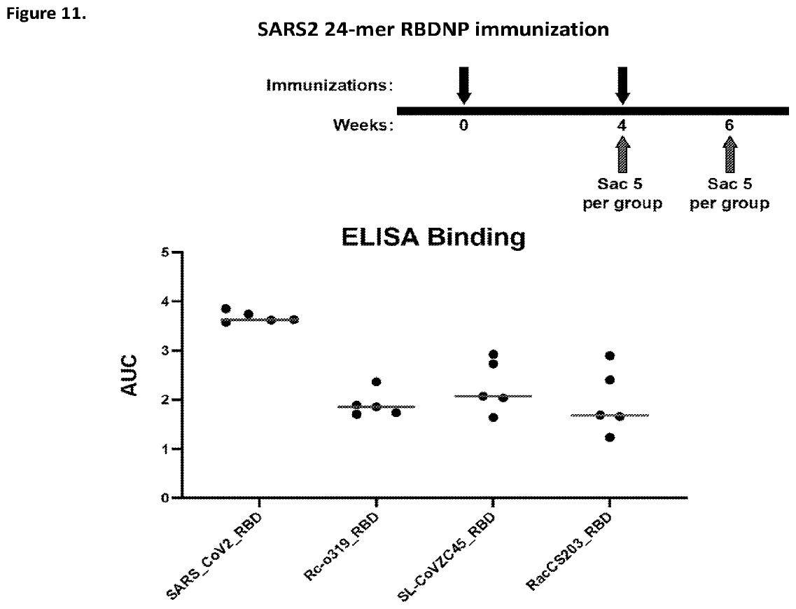

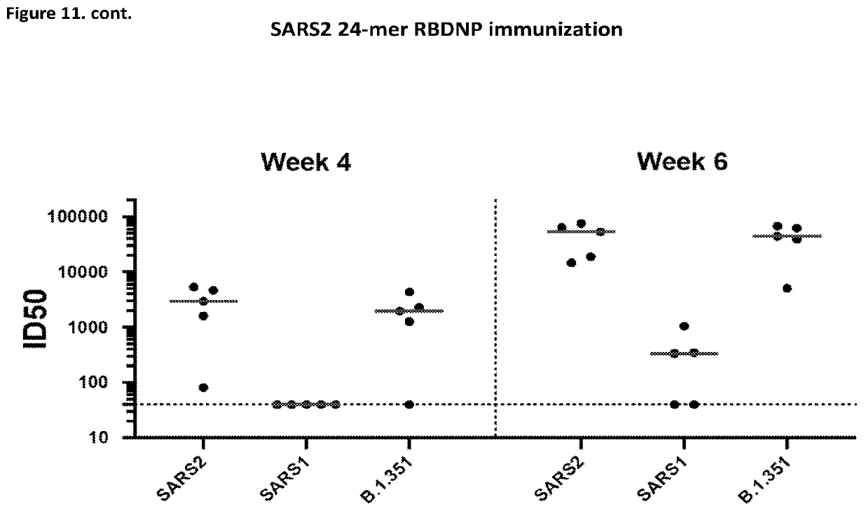

FIG. 11 shows the results of immunization with a SARS-CoV-2 RBD-NP 24mer construct. BALB/c mice (n=10) were immunized with 10 microg of a SARS-CoV-2 RBD-24mer-14 (SEQ ID NO: 113) with 100 microL Addavax adjuvant via subcutaneous injection in the scruff of the neck using a total injection volume of 200 microL. The mice were primed at week 0 and either sacrificed (n=5) or boosted with the same regimen at week 4. All remaining mice were sacrificed at week 5. ELISA binding to SARS_CoV2_RBD, Rc-o319_RBD (SEQ ID NO:154), SL-CoVZC45 (SEQ ID NO:155), RBD and RacCS203_RBD (SEQ ID NO:156) was assessed at week 6. The immunization protocol induced the formation of high affinity antibodies against the SARS_CoV2_RBS. The immune response was specific for SARS_CoV2_RBD as reflected by the reduced binding to the control Rc-o319_RBD, SL-CoVZC45 RBD and RacCS203_RBD. Serum neutralization activity against SARS_CoV2, SARS_CoV1 and B1.351 variant SARS_CoV2 was measured at weeks 4 and 6. The immunized animals developed a high titer of neutralizing antibodies against SARS_CoV2. The neutralizing antibodies were also effective against the B1.351 variant. The specificity of the immune response is demonstrated by the fact that the neutralization was considerably weaker to SARS_CoV1 control.

While the invention has been described in connection with what is presently considered to be the most practical and preferred embodiments, it is to be understood that the invention is not to be limited to the described embodiments, but on the contrary, is intended to cover various modifications and equivalent arrangements included within the spirit and scope of the appended claims.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

What is claimed is:

1. A fusion polypeptide comprising
a) at least one viral polypeptide comprising a SARS-CoV spike protein (S), a SARS-CoV-2 spike protein (S), or an immunogenic fragment thereof; and
b) an amino acid sequence that targets the fusion polypeptide to the cell surface or a self-assembling domain capable of forming a nanoparticle.

2. The fusion polypeptide of claim 1 comprising a SARS-CoV-2 spike protein (S) or an immunogenic fragment thereof.

3. The fusion polypeptide of claim 2, wherein the SARS-CoV-2 spike protein (S) comprises an amino acid sequence having at least 70%, at least 80%, at least 85%, at least 90%, at least 95%, at least 97%, at least 98% at least 99% or at least 100% identity with SEQ ID NO:51.

4. The fusion polypeptide of claim 2 or claim 3, wherein the SARS-CoV-2 spike protein (S) or an immunogenic fragment thereof comprises a trimerized SARS-CoV-2 receptor-binding domain.

5. The fusion polypeptide of claim 2 or claim 3, wherein the SARS-CoV-2 spike protein (S) or an immunogenic fragment thereof comprises prefusion stabilized membrane-anchored SARS-CoV-2 full-length spike protein.

6. The fusion polypeptide of claim 2 or claim 3, wherein the SARS-CoV-2 spike protein (S) or an immunogenic fragment thereof comprises a prefusion stabilized SARS-CoV-2 spike protein.

7. The fusion polypeptide of claim 2 or claim 3, wherein the SARS-CoV-2 spike protein (S) or an immunogenic fragment thereof comprises the receptor binding domain of the SARS-CoV-2 spike protein.

8. The fusion polypeptide of claim 7, wherein the receptor binding domain comprises the amino acid sequence of SEQ ID NO:32 or SEQ ID NO:33.

9. The fusion polypeptide of claim 7, wherein the receptor binding domain comprises an amino acid sequence having at least 70%, at least 80%, at least 85%, at least 90%, at least 95%, at least 97%, at least 98% at least 99% or at least 100% identity with SEQ ID NO:32 or SEQ ID NO:33.

10. The fusion polypeptide of any one of claims 7 to 9, wherein receptor binding domain comprises one or more engineered glycosylation site, wherein the engineered glycosylation site comprises the amino acid sequence of NXS or NXT, wherein X is not proline.

11. The fusion polypeptide of claim 10, wherein the one or more engineered glycosylation site is at an amino acid position corresponding to position 346, 357, 360, 370, 381, 386, 394, 428, 444, 458, 468, 481, 518, and/or 522 of SEQ ID NO:51.

12. The fusion polypeptide of claim 10, wherein the one or more engineered glycosylation site is at an amino acid position corresponding to position 357, 360, 381, 386, 394, 428, 518, and/or 522 of SEQ ID NO:51.

13. The fusion polypeptide of any one of claims 10 to 12, wherein the receptor binding domain comprises 1, 2, 3, 4, 5, 6, 7, or 8 engineered glycosylation sites.

14. The fusion polypeptide of any one of claims 10 to 12, wherein the receptor binding domain comprises an engineered glycosylation site at the amino acid positions corresponding to
a) positions 357, 381, 386, 394, and 528 of SEQ ID NO:51;
b) positions 357, 394, 428, 518, and 522 of SEQ ID NO:51;
c) positions 357, 394, 428, and 518 of SEQ ID NO:51;
d) positions 357, and 518 of SEQ ID NO:51;
e) positions 357, 386, and 518 of SEQ ID NO:51;
f) positions 357, 386, 394, 428, and 518 of SEQ ID NO:51;
g) positions 386, 394, 518, and 522 of SEQ ID NO:51;
h) positions 357, 381, 386, 394, 428, 518, and 522 of SEQ ID NO:51;
i) positions 357, 386, 394, 428, 518, and 522 of SEQ ID NO:51;
j) positions 357, 381, 394, and 428 of SEQ ID NO:51; or
k) positions 357, 381, 394, and 518 of SEQ ID NO:51.

15. The fusion polypeptide of any one of claims 10 to 12, wherein the receptor binding domain comprises an engineered glycosylation site at the amino acid positions corresponding to
a) positions 357, 386, 394, 428, and 518 of SEQ ID NO:51;
b) positions 357, 386, 394, and 518 of SEQ ID NO:51;
c) positions 357, 386, and 518 of SEQ ID NO:51.

16. The fusion polypeptide of any one of claims 10 to 12, wherein the receptor binding domain comprises an engineered glycosylation site at the amino acid positions corresponding to positions 357, 386, 394, 428, and 518 of SEQ ID NO:51.

17. The fusion polypeptide of any one of claims 10 to 12, wherein the receptor binding domain comprises an engineered glycosylation site at the amino acid positions corresponding to positions 357, and 518 of SEQ ID NO:51.

18. The fusion polypeptide of any one of claims 10 to 12, wherein the receptor binding domain comprises an engineered glycosylation site at the amino acid positions corresponding to
a) positions 357, and 518 of SEQ ID NO:51;
b) positions 346, 357, 428, and 518 of SEQ ID NO:51;
c) positions 357, 386, 394, and 518 of SEQ ID NO:51;
d) positions 346, 357, 386, 428, and 518 of SEQ ID NO:51;
e) positions 357, 428, and 518 of SEQ ID NO:51;
f) positions 357, 386, 428, and 518 of SEQ ID NO:51; or
g) positions 357, 394, and 518 of SEQ ID NO:51.

19. The fusion polypeptide of any one of claims 1 to 18, wherein the fusion polypeptide comprises an amino acid sequence that targets the fusion polypeptide to the cell surface.

20. The fusion polypeptide of claim 18, wherein the amino acid sequence that targets the fusion polypeptide to the cell surface comprises a GPI anchor signal sequence.

21. The fusion polypeptide of claim 19, wherein the amino acid sequence that targets the fusion polypeptide to the cell surface comprises a transmembrane domain.

22. The fusion polypeptide of claim 19, wherein the receptor binding domain comprises the amino acid sequence of SEQ ID NO:32.

23. The fusion polypeptide of claim 21 or claim 22, wherein the transmembrane domain comprises
a) an HIV Env transmembrane domain,
b) a SARS-CoV-2 transmembrane domain, or
c) a VSV-G transmembrane domain.

24. The fusion polypeptide of claim 23, wherein
a) the HIV Env transmembrane domain comprises the amino acid sequence of SEQ ID NO:6,
b) the SARS-CoV-2 transmembrane domain comprises the amino acid sequence of SEQ ID NO:7, and
c) the VSV-G transmembrane domain comprises the amino acid sequence of SEQ ID NO:8.

25. The fusion polypeptide of claim 21 or claim 22, wherein the transmembrane domain comprises a VSV-G transmembrane domain.

26. The fusion polypeptide of claim 25, wherein the VSV-G transmembrane domain comprises the amino acid sequence of SEQ ID NO:8.

27. The fusion polypeptide of any one of claims 21 to 26, wherein the viral polypeptide and the transmembrane domain are directly linked.

28. The fusion polypeptide of any one of claims 21 to 26, wherein the viral polypeptide and the transmembrane domain are separated by a linker peptide.

29. The fusion polypeptide of claim 28, wherein the linker comprises no more than 10 or no more than 5 amino acid residues.

30. The fusion polypeptide of claim 28 or claim 29, wherein the linker comprises one or more repeats of the GGS (SEQ ID NO:43) or GGGS (SEQ ID NO:44) sequence.

31. The fusion polypeptide of claim 28 or claim 29, wherein the linker comprises the amino acid sequence of GGS (SEQ ID NO:43), GGSGGS (SEQ ID NO:45), GGSGGSGGS (SEQ ID NO:46), GGGS (SEQ ID NO: 44), GGGSGGGS (SEQ ID NO:47), or GGGSGGGSGGGS (SEQ ID NO:34).

32. The fusion polypeptide of any one of claims 21 to 31, wherein the viral polypeptide is closer to the N terminus than the transmembrane domain.

33. The fusion polypeptide of any one of claims 1 to 18, wherein the fusion polypeptide comprises a self-assembling domain capable of forming a nanoparticle.

34. The fusion polypeptide of claim 33, wherein the wherein the receptor binding domain comprises the amino acid sequence of SEQ ID NO:33.

35. The fusion polypeptide of claim 33 or claim 34, wherein the self-assembling domain comprises a type II 3-Dehydroquinase, ferritin or lumazine synthase.

36. The fusion polypeptide of claim 33 or claim 34, wherein the self-assembling domain comprises a Thermus thermophilus, Mycobacterium tuberculosis, Streptomyces coelicolor, Acinetobacter baumannii, Yersinia pestis, Bacillus subtilis, Proprionibacterium acnes, Acidithiobacillus caldus, Zymomonas mobilus, Helicobacter pylori, Pseudomonas aeruginosa, Candida albicans, or Psychromonas ingrahamii type II 3-Dehydroquinase polypeptide.

37. The fusion polypeptide of claim 33 or claim 34, wherein the self-assembling domain comprises a Thermus thermophilus type II 3-Dehydroquinase polypeptide.

38. The fusion polypeptide of claim 37, wherein the Thermus thermophilus type II 3-Dehydroquinase polypeptide comprises an amino acid sequence having at least 70%, at least 80%, at least 85%, at least 90%, at least 95%, at least 97%, at least 98% at least 99% or at least 100% identity with SEQ ID NO:48.

39. The fusion polypeptide of claim 37, wherein the Thermus thermophilus type II 3-Dehydroquinase polypeptide comprises the amino acid sequence of SEQ ID NO:48.

40. The fusion polypeptide of any one of claims 36 to 39, wherein the 3-Dehydroquinase polypeptide comprises one or more engineered glycosylation site, wherein the engineered glycosylation site comprises the amino acid sequence of NXS or NXT, wherein X is not proline.

41. The fusion polypeptide of claim 40, wherein the one or more engineered glycosylation site is at an amino acid position corresponding to position 1, 25, 32, 49, and/or 63 of SEQ ID NO:52.

42. The fusion polypeptide of claim 40 or claim 41, wherein the 3-Dehydroquinase polypeptide comprises 1, 2, 3, 4, or 5 engineered glycosylation sites.

43. The fusion polypeptide of any one of claims 40 to 42, wherein the 3-Dehydroquinase polypeptide comprises the amino acid sequence of SEQ ID NO:52.

44. The fusion polypeptide of any one of claims 33 to 43, wherein the fusion polypeptide comprises from the N terminus to the C terminus VP-SAD, SAD-VP, VP-SAD-VP, wherein VP and SAD corresponds to the at least one viral polypeptide, and self-assembling domain, respectively.

45. The fusion polypeptide of claim 44, wherein the viral polypeptide comprises the receptor binding domain of the SARS-CoV-2 spike protein comprising one or more engineered glycosylation site.

46. The fusion polypeptide of claim 44 or claim 45, wherein the VP and SAD are directly linked.

47. The fusion polypeptide of claim 44 or claim 45, wherein the fusion polypeptide comprises one or more linkers linking the VP and SAD.

48. The fusion polypeptide of claim 44 or claim 45, wherein the fusion polypeptide comprises one or more linkers linking the VP and SAD.

49. The fusion polypeptide of claim 47 and claim 48, wherein the one or more linker independently comprises no more than 10 or no more than 5 amino acid residues.

50. The fusion polypeptide of any one of claims 47 to 49, wherein the one or more linker independently comprises one or more repeats of the GGS (SEQ ID NO:43) or GGGS (SEQ ID NO:44) sequence.

51. The fusion polypeptide of any one of claims 47 to 50, wherein the one or more linker independently comprises the amino acid sequence of GGS (SEQ ID NO:43), GGSGGS (SEQ ID NO:45), GGSGGSGGS (SEQ ID NO:46), GGGS (SEQ ID NO: 44), GGGSGGGS (SEQ ID NO:47), or GGGSGGGSGGGS (SEQ ID NO:34).

52. The fusion polypeptide of any one of claims 33 to 51, wherein the fusion polypeptide further comprises a His tag.

53. The fusion polypeptide of any one of claims 33 to 51, wherein the fusion polypeptide further comprises the amino acid sequence of HGKHGK (SEQ ID NO:35).

54. The fusion polypeptide of claim 52 or claim 53, wherein the His tag or the HGKHGK (SEQ ID NO:35) sequence is at the C terminal end of the fusion polypeptide.

55. The fusion polypeptide of any one of claims 1 to 54, wherein the fusion polypeptide further comprises at least one immunogenic polypeptide comprising one or more MHC class II T cell epitope.

56. The fusion polypeptide of any one of claim 55, wherein the MHC class II T cell epitope comprises the amino acid sequence of AKFVAAWTLKAAA (SEQ ID NO:36).

57. The fusion polypeptide of any one of claim 55, wherein the MHC class II T cell epitope comprises an amino acid sequence selected from the group consisting of
a)
(SEQ ID NO: 37)
ATPHFDYIASEVSKG comprising 0, 1, 2, 3, 4, or 5 substitutions,
b)
(SEQ ID NO: 38)
FGVITADTLEQAIER comprising 0, 1, 2, 3, 4, or 5 substitutions,
c)
(SEQ ID NO: 39)
FDYIASEVSKGLADL comprising 0, 1, 2, 3, 4, or 5 substitutions,
d)
(SEQ ID NO: 40)
ATPHFDYIASEVSKGLADL comprising 0, 1, 2, 3, 4, or 5 substitutions,
e) 9, 10, 11, 12, 13, 14, or 15 consecutive residues of
(SEQ ID NO: 37)
ATPHFDYIASEVSKG,
f) 9, 10, 11, 12, 13, 14, or 15 consecutive residues of
(SEQ ID NO: 38)
FGVITADTLEQAIER,
g) 9, 10, 11, 12, 13, 14, or 15 consecutive residues of
(SEQ ID NO: 39)
FDYIASEVSKGLADL,
and
h) 10, 11, 12, 13, 14, 15, 16, 17, 18, or 19 consecutive residues of
(SEQ ID NO: 40)
ATPHFDYIASEVSKGLADL

58. The fusion polypeptide of claim 55, wherein the MHC class II T cell epitope comprises an amino acid sequence selected from the group consisting of
a) 9, 10, 11, 12, 13, 14, or 15 consecutive residues of
(SEQ ID NO: 37)
ATPHFDYIASEVSKG,
b) 9, 10, 11, 12, 13, 14, or 15 consecutive residues of
(SEQ ID NO: 38)
FGVITADTLEQAIER,
c) 9, 10, 11, 12, 13, 14, or 15 consecutive residues of
(SEQ ID NO: 39)
FDYIASEVSKGLADL,
and
d) 10, 11, 12, 13, 14, 15, 16, 17, 18, or 19 consecutive residues of
(SEQ ID NO: 40)
ATPHFDYIASEVSKGLADL.

59. The fusion polypeptide of claim 55, wherein the MHC class II T cell epitope comprises an amino acid sequence selected from the group consisting of
a)
(SEQ ID NO: 37)
ATPHFDYIASEVSKG,
b)
(SEQ ID NO: 38)
FGVITADTLEQAIER,
c)
(SEQ ID NO: 39)
FDYIASEVSKGLADL,
and
d)
(SEQ ID NO: 40)
ATPHFDYIASEVSKGLADL

60. The fusion polypeptide of claim 55, wherein the immunogenic polypeptide comprises at least 2 MHC class II T cell epitopes.

61. The fusion polypeptide of claim 60, wherein the at least 2 MHC class II T cell epitopes comprise the amino acid sequences of
a)
(SEQ ID NO: 37)
ATPHFDYIASEVSKG comprising 0, 1, 2, 3, 4, or 5 substitutions;
and
b)
(SEQ ID NO: 38)
FGVITADTLEQAIER comprising 0, 1, 2, 3, 4, or 5 substitutions.

62. The fusion polypeptide of claim 60, wherein the at least 2 MHC class II T cell epitopes comprise the amino acid sequences of
a)
(SEQ ID NO: 37)
ATPHFDYIASEVSKG;
and
b)
(SEQ ID NO: 38)
FGVITADTLEQAIER

63. The fusion polypeptide of claim 60, wherein the at least 2 MHC class II T cell epitopes comprise the amino acid sequences of
a)
(SEQ ID NO: 40)
ATPHFDYIASEVSKGLADL comprising 0, 1, 2, 3, 4, or 5 substitutions;
and
b)
(SEQ ID NO: 38)
FGVITADTLEQAIER comprising 0, 1, 2, 3, 4, or 5 substitutions.

64. The fusion polypeptide of claim 60, wherein the at least 2 MHC class II T cell epitopes comprise the amino acid sequences of
a)
(SEQ ID NO: 40)
ATPHFDYIASEVSKGLADL;
and
b)
(SEQ ID NO: 38)
FGVITADTLEQAIER.

65. The fusion polypeptide of any one of claims 60 to 64, wherein the at least 2 MHC class II T cell epitopes are directly linked in any order.

66. The fusion polypeptide of any one of claims 60 to 64, wherein the at least 2 MHC class II T cell epitopes are in any order and are separated by a linker peptide.

67. The fusion polypeptide of claim 66, wherein the linker comprises no more than 10 or no more than 5 amino acid residues.

68. The fusion polypeptide of claim 66 or claim 67, wherein the linker comprises one or more repeats of the GGS (SEQ ID NO:43) or GGGS (SEQ ID NO:44) sequence.

69. The fusion polypeptide of claim 66 or claim 67, wherein the linker comprises the amino acid sequence of GGS (SEQ ID NO:43), GGSGGS (SEQ ID NO:45), GGSGGSGGS (SEQ ID NO:46), GGGS (SEQ ID NO: 44), GGGSGGGS (SEQ ID NO:47), or GGGSGGGSGGGS (SEQ ID NO:34).

70. The fusion polypeptide of claim 55, wherein the immunogenic polypeptide comprises an amino acid sequence selected from the group consisting of
a)
(SEQ ID NO: 40)
ATPHFDYIASEVSKGLADL comprising 0, 1, 2, 3, 4, or 5 substitutions;
b)
(SEQ ID NO: 40)
ATPHFDYIASEVSKGLADL;
c)
(SEQ ID NO: 41)
ATPHFDYIASEVSKGLADLGGSFGVITADTLEQAIER comprising 0, 1, 2,
3, 4, or 5 substitutions;
d) amino acid sequence having at least 70%, at least 80%, at least 85%, at least 90%, at least 95%, at least 97%, at least 98% at least 99% or at least 100% identity with
(SEQ ID NO: 41)
ATPHFDYIASEVSKGLADLGGSFGVITADTLEQAIER;
e)
(SEQ ID NO: 41)
ATPHFDYIASEVSKGLADLGGSFGVITADTLEQAIER;
f)
(SEQ ID NO: 42)
ATPHFDYIASEVSKGLADLSLELRKPITFGVITADTLEQAIER
comprising 0, 1, 2, 3, 4, or 5 substitutions;
g) amino acid sequence having at least 70%, at least 80%, at least 85%, at least 90%, at least 95%, at least 97%, at least 98% at least 99% or at least 100% identity with
(SEQ ID NO: 42)
ATPHFDYIASEVSKGLADLSLELRKPITFGVITADTLEQAIER;
and
h)
(SEQ ID NO: 42)
ATPHFDYIASEVSKGLADLSLELRKPITFGVITADTLEQAIER.

71. The fusion polypeptide of claim 55, wherein the immunogenic polypeptide comprises an amino acid sequence selected from the group consisting of
a)
(SEQ ID NO: 40)
ATPHFDYIASEVSKGLADL;
b)
(SEQ ID NO: 41)
ATPHFDYIASEVSKGLADLGGSFGVITADTLEQAIER;
and
c)
(SEQ ID NO: 42)
ATPHFDYIASEVSKGLADLSLELRKPITFGVITADTLEQAIER.

72. The fusion polypeptide of claim 55, wherein the immunogenic polypeptide comprises the amino acid sequence of ATPHFDYIASEVSKGLADL (SEQ ID NO:40).

73. The fusion polypeptide of claim 55, wherein the immunogenic polypeptide comprises the amino acid sequence of ATPHFDYIASEVSKGLADLGGSFGVITADTLEQAIER (SEQ ID NO:41).

74. The fusion polypeptide of any one of claims 55 to 73, wherein the fusion polypeptide comprises from the N terminus to the C terminus VP-IP-TM or VP-TM-IP, wherein VP, IP and TM corresponds to the at least one viral polypeptide, at least one immunogenic polypeptide, and transmembrane domain, respectively.

75. The fusion polypeptide of claim 74, wherein the viral polypeptide comprises the receptor binding domain of the SARS-CoV-2 spike protein comprising one or more engineered glycosylation site.

76. The fusion polypeptide of claim 74 or claim 75, wherein the VP, IP and TM are directly linked.

77. The fusion polypeptide of claim 74 or claim 75, wherein the fusion polypeptide comprises one or more linkers linking the VP, IP and/or TM.

78. The fusion polypeptide of claim 74 or claim 75, wherein the fusion polypeptide comprises linkers linking the VP, IP and TM.

79. The fusion polypeptide of claim 77 and claim 78, wherein the one or more linker or linkers independently comprise no more than 10 or no more than 5 amino acid residues.

80. The fusion polypeptide of any one of claims 77 to 79, wherein the one or more linker or linkers independently comprise one or more repeats of the GGS (SEQ ID NO:43) or GGGS (SEQ ID NO:44) sequence.

81. The fusion polypeptide of any one of claims 77 to 80, wherein the one or more linker or linkers independently comprise the amino acid sequence of GGS (SEQ ID NO:43), GGSGGS (SEQ ID NO:45), GGSGGSGGS (SEQ ID NO:46), GGGS (SEQ ID NO: 44), GGGSGGGS (SEQ ID NO:47), or GGGSGGGSGGGS (SEQ ID NO:34).

82. The fusion polypeptide of any one of claims 55 to 73, wherein the fusion polypeptide comprises from the N terminus to the C terminus VP-IP-SAD, SAD-IP-VP, VP-IP-SAD-IP-VP, wherein VP, IP and SAD corresponds to the at least one viral polypeptide, at least one immunogenic polypeptide, and self-assembling domain, respectively.

83. The fusion polypeptide of claim 82, wherein the viral polypeptide comprises the receptor binding domain of the SARS-CoV-2 spike protein comprising one or more engineered glycosylation site.

84. The fusion polypeptide of claim 82 or claim 83, wherein the VP, IP and SAD are directly linked.

85. The fusion polypeptide of claim 82 or claim 83, wherein the fusion polypeptide comprises one or more linker linking the VP, IP and/or SAD.

86. The fusion polypeptide of claim 82 or claim 83, wherein the fusion polypeptide comprises linkers linking the VP, IP and SAD.

87. The fusion polypeptide of claim 85 and claim 86, wherein the one or more linker independently comprises no more than 10 or no more than 5 amino acid residues.

88. The fusion polypeptide of any one of claims 85 to 87, wherein the one or more linker independently comprises one or more repeats of the GGS (SEQ ID NO:43) or GGGS (SEQ ID NO:44) sequence.

89. The fusion polypeptide of any one of claims 85 to 88, wherein the one or more linker independently comprises the amino acid sequence of GGS (SEQ ID NO:43), GGSGGS (SEQ ID NO:45), GGSGGSGGS (SEQ ID NO:46), GGGS (SEQ ID NO: 44), GGGSGGGS (SEQ ID NO:47), or GGGSGGGSGGGS (SEQ ID NO:34).

90. The fusion polypeptide of claim 21 comprising an amino acid sequence selected from the group consisting of SEQ ID NO:57, SEQ ID NO:59, SEQ ID NO:61, SEQ ID NO:63, SEQ ID NO:65, SEQ ID NO:67, SEQ ID NO:69, and SEQ ID NO:71.

91. The fusion polypeptide of claim 33 comprising an amino acid sequence selected from the group consisting of SEQ ID NO: 73-152 or 153.

92. The fusion polypeptide of any one of claims 1 to 91 further comprising a signal peptide.

93. The fusion polypeptide of claim 92, wherein the signal peptide comprises the amino acid sequence of
(SEQ ID NO: 5)
MGILPSPGMPALLSLVSLLSVLLMGCVAETG.

94. An isolated polynucleotide encoding the fusion polypeptide of any one of claims 1 to 93.

95. The polynucleotide of claim 94 that is DNA.

96. The polynucleotide of claim 94 that is RNA.

97. The polynucleotide of claim 96, wherein the RNA is mRNA comprising modified ribonucleotides.

98. A vector comprising the polynucleotide of claim 94.

99. A host cell comprising the polynucleotide of claim 94 or the vector of claim 98.

100. The host cell of claim 99 which is a CHO cell or a HEK293 cell.

101. A recombinant virus comprising the polynucleotide of claim 94.

102. An immunogenic composition comprising the fusion polypeptide of any one of claims 1 to 93, the polynucleotide of any one of claims 94 to 97, the vector of claim 98, or the recombinant virus of claim 101.

103. The immunogenic composition of claim 102, further comprising an adjuvant, wherein optionally the adjuvant comprises AS01B, AS03, alum, SMNP, ISCOMs, CpG, and combinations thereof.

104. The immunogenic composition of claim 103, wherein the immunogenic composition comprises the fusion polypeptide of any one of claims 1 to 93.

105. The immunogenic composition of any one of claims 102 to 104, wherein the immunogenic composition is capable of eliciting an increased immune response in a subject compared to the immune response elicited by a reference immunogenic composition comprising or encoding a reference fusion polypeptide not comprising the one or more MHC class II T cell epitope.

106. The immunogenic composition of claim 105, wherein the increased immune response is an increased humoral response.

107. The immunogenic composition of claim 105, wherein the increased immune response is an increased cellular immune response.

108. The immunogenic composition of any one of claims 105 to 107, wherein the subject is a mouse or a cynomolgus monkey.

109. A pharmaceutical composition comprising the fusion polypeptide of any one of claims 1 to 93, the polynucleotide of any one of claims 94 to 97, the vector of claim 98, or the recombinant virus of claim 101, and a pharmaceutically acceptable excipient.

110. A method of vaccinating a subject comprising administering a therapeutically effective amount of the fusion polypeptide of any one of claims 1 to 93, the polynucleotide of any one of claims 94 to 97, the vector of claim 98, or the recombinant virus of claim 101, the immunogenic composition of any one of claims 102 to 108, or the pharmaceutical composition of claim 109 to the subject.

111. The method of claim 110 comprising administering a therapeutically effective amount of the fusion polypeptide of any one of claims 1 to 93.

112. The method of claim 110 comprising administering a therapeutically effective amount of the polynucleotide of any one of claims 94 to 97.

113. The method of claim 112, wherein the polynucleotide comprises an mRNA comprising modified ribonucleotides.

114. A method of inducing an immune response in a subject comprising administering an effective amount of the fusion polypeptide of any one of claims 1 to 93, the polynucleotide of any one of claims 94 to 97, the vector of claim 98, or the recombinant virus of claim 101, the immunogenic composition of any one of claims 102 to 108, or the pharmaceutical composition of claim 109 to the subject.

115. The method of claim 114, wherein the immune response is a viral antigen-specific immune response.

116. The method of claim 114 comprising administering an effective amount of the fusion polypeptide of any one of claims 1 to 93.

117. The method of claim 114 comprising administering an effective amount of the polynucleotide of any one of claims 94 to 97.

118. The method of claim 117, wherein the polynucleotide comprises an mRNA comprising modified ribonucleotides.

119. A method of treating a viral infection in a subject comprising administering a therapeutically effective amount of the fusion polypeptide of any one of claims 1 to 93, the polynucleotide of any one of claims 94 to 97, the vector of claim 98, or the recombinant virus of claim 101, the immunogenic composition of any one of claims 102 to 108, or the pharmaceutical composition of claim 109 to the subject.

120. The method of claim 119, wherein the viral infection is a SARS-CoV-2 infection.

121. The method of claim 119 comprising administering a therapeutically effective amount of the fusion polypeptide of any one of claims 1 to 93.

122. The method of claim 119 comprising administering a therapeutically effective amount of the polynucleotide of any one of claims 94 to 97.

123. The method of claim 122, wherein the polynucleotide comprises an mRNA comprising modified ribonucleotides.

124. The method of any one of claims 110 to 123, wherein the subject is a human.

125. A method of producing the fusion polypeptide according to any one of claims 1 to 93, comprising culturing the host cell of claim 99 under suitable conditions to produce the fusion polypeptide.

126. The method of claim 125, wherein the host cell comprises a CHO cell or a HEK293 cell.

127. A method of producing the isolated polynucleotide according to claim 97, comprising producing the mRNA through chemical synthesis or in vitro translation.
